(19) United States
(12) Patent Application Publication
(10) Pub. No.: US 2024/0011045 A1
(43) Pub. Date: Jan. 11, 2024

(54) PROTEINS FOR REGULATION OF SYMBIOTIC NODULE ORGAN IDENTITY

(71) Applicant: Cambridge Enterprise Limited, Cambridge (GB)

(72) Inventors: Giles Edward Dixon Oldroyd, London (GB); Katharina Schiessl, Norwich (GB); Tak Lee, Cologne (DE); Min-yao Jhu, Cambridge (GB)

(21) Appl. No.: 18/320,150

(22) Filed: May 18, 2023

Related U.S. Application Data
(60) Provisional application No. 63/343,950, filed on May 19, 2022.

(51) Int. Cl. C12N 15/82; C07K 14/415

(52) U.S. Cl. CPC C12N 15/8261; C07K 14/415

(57) ABSTRACT

The present invention provides novel DNA molecules and constructs, including their nucleotide sequences, useful for expressing proteins in plants to promote symbiotic infection. The invention also provides plants and plant cells transgenic plants, plant cells, plant parts, seeds, and commodity products comprising the DNA molecules operably linked to heterologous transcribable polynucleotides, along with methods of their use.

### CROSS REFERENCE TO RELATED APPLICATIONS

This application claims the benefit of U.S. Provisional Application No. 63/343,950, filed on May 19, 2022, the entire content of which is hereby incorporated herein by reference.

### INCORPORATION OF SEQUENCE LISTING

The sequence listing that is contained in the file named “AGOE007US_ST26.xml” containing 94 sequences, which is 121 KB (as measured in Microsoft Windows®) and was created on May 18, 2023, is filed herewith by electronic submission and is incorporated by reference herein.

### FIELD OF THE INVENTION

The invention relates to the field of plant molecular biology and plant genetic engineering, DNA molecules useful for modulating gene expression in plants, and proteins useful for improving agronomic performance.

### BACKGROUND

Many of the world's farmers face pressure from nitrogen-deficient or phosphate-deficient soils which can result in low yield or plant death. Symbiotic nitrogen-fixing bacteria can improve plant biomass under low-nitrogen conditions. In order to initiate this process, legumes grow specialized root nodules to host beneficial nitrogen-fixing bacteria that provide the plant with ammonia in exchange for carbon. These symbiotic nodules are distinct from lateral roots in morphology and function with nodules comprising cells that accommodate nitrogen-fixing rhizobial bacteria. However, the factors behind determination of nodule organ identity are not well understood. Therefore, methods for promoting nodule formation for symbiotic infection in both legume and non-legume plants are needed to provide farmers with crop plants exhibiting improved agronomic performance under nitrogen-limited conditions.

### SUMMARY OF THE INVENTION

In one aspect the present disclosure provides a recombinant DNA molecule comprising a heterologous promoter operably linked to a polynucleotide segment encoding a light sensitive short hypocotyl protein or fragment thereof, wherein: a. said protein comprises the amino acid sequence of SEQ ID NO: 2 or 4; b. said protein comprises an amino acid sequence having at least 85%, or 90%, or 95%, or 98% or 99%, or about 100% amino acid sequence identity to SEQ ID NO: 2 or 4; or c. said polynucleotide segment hybridizes under stringent hybridization conditions to a polynucleotide having the nucleotide sequence of SEQ ID NO: 1, 3, 5, 6, 7, or 8. In some embodiments, recombinant DNA molecules provided are expressed in a plant cell to produce an increase in intercellular cortical infection, an increase in intracellular colonization by nitrogen-fixing bacteria, or an increase in nitrogen-fixation by bacteria. In further embodiments, recombinant DNA molecules provided are in operable linkage with a vector, and said vector is selected from the group consisting of a plasmid, phagemid, bacmid, cosmic, and a bacterial or yeast artificial chromosome. Recombinant DNA molecules disclosed may be present within a host cell, wherein said host cell is selected from the group consisting of a bacterial cell and a plant cell. For example, said bacterial host cell may be from a genus of bacteria selected from the group consisting of: Agrobacterium, Rhizobium, Bacillus, Brevibacillus, Escherichia, Pseudomonas, Klebsiella, Pantoea, and Erwinia. In more specific examples, said Bacillus is Bacillus cereus or Bacillus thuringiensis, said Brevibacillus is a Brevibacillus laterosperous, or said Escherichia is a Escherichia coli. In other examples, said plant cell may be from a dicotyledonous or a monocotyledonous plant cell, such as for example a plant cell selected from the group consisting of an alfalfa, almond, Bambara groundnut, banana, barley, bean, black currant, broccoli, blackberry, brassica, cabbage, canola, carrot, cassava, castor, cauliflower, celery, chickpea, Chinese cabbage, citrus, coconut, coffee, corn, clover, cotton, cowpea, cucurbit, cucumber, Douglas fir, eggplant, eucalyptus, flax, forage legume, garlic, grape, hemp, hops, indigo, leek, legume, legume trees, lentil, lettuce, Loblolly pine, lotus, lupin, millets, melons, Medicago spp., nut, oat, olive, onion, ornamental, palm, pasture grass, pea, peanut, pepper, pigeon pea, pine, potato, poplar, pumpkin, pulses, Radiata pine, radish, rapeseed, raspberry, red currant, rice, rootstocks, rye, safflower, shrub, sorghum, Southern pine, soybean, spinach, squash, strawberry, sugar beet, sugarcane, sunflower, sweet gum, sweet potato, switchgrass, tea, tobacco, tomato, triticale, turf grass, walnut, watermelon, wheat, and yam plant cell. In another aspect, a plant or part thereof is provided comprising the recombinant DNA molecules described herein. In certain embodiments, said plant may be a monocot plant or a dicot plant, for example, a plant selected from the group consisting of an alfalfa, almond, Bambara groundnut, banana, barley, bean, black currant, broccoli, cabbage, blackberry, brassica, canola, carrot, cassava, castor, cauliflower, celery, chickpea, Chinese cabbage, citrus, coconut, coffee, corn, clover, cotton, cowpea, a cucurbit, cucumber, Douglas fir, eggplant, eucalyptus, flax, forage legume, garlic, grape, hemp, hops, indigo, leek, legume, legume trees, lentil, lettuce, Loblolly pine, lotus, lupin, millets, melons, Medicago spp., nut, oat, olive, onion, ornamental, palm, pasture grass, pea, peanut, pepper, pigeon pea, pine, potato, poplar, pumpkin, pulses, Radiata pine, radish, rapeseed, raspberry, red currant, rice, rootstocks, rye, safflower, shrub, sorghum, Southern pine, soybean, spinach, squash, strawberry, sugar beet, sugarcane, sunflower, sweet gum, sweet potato, switchgrass, tea, tobacco, tomato, triticale, turf grass, walnut, watermelon, wheat, and yam. In certain embodiments, plants or parts thereof of as described herein exhibit varying expression of a polynucleotide segment encoding a light sensitive short hypocotyl protein over a 24-hour period. For example, a plant or part thereof as described may express a polynucleotide segment encoding a light sensitive short hypocotyl protein at an increased level during the first 12 hours of a 12 hour/12 hour light/dark cycle. In another example, a plant or part thereof as described may express a polynucleotide segment encoding a light sensitive short hypocotyl protein at an increased level during the first 6 hours of a 12 hour/12 hour light/dark cycle. In yet further embodiments, transgenic seeds are provided comprising the recombinant DNA molecules described herein. In another aspect, methods of producing progeny seed are provided comprising the recombinant DNA molecules provided herein, the methods comprising: a. planting a first seed comprising a recombinant DNA molecule provided; b. growing a plant from the seed of step a; and c. harvesting the progeny seed from the plants, wherein said harvested seed comprises said recombinant DNA molecule. Further aspects provide plants susceptible to intercellular cortical infection or intracellular colonization by nitrogen-fixing bacteria, wherein the cells of said plant comprise the recombinant DNA molecules described herein. Also provided are methods for increasing intercellular cortical infection or intracellular colonization by nitrogen-fixing bacteria in a plant, said methods comprising: a. expressing a light sensitive short hypocotyl protein or fragment thereof having at least 70%, or 80%, or 90%, or 95%, or 99%, or about 100% sequence identity to SEQ ID NO: 2 or 4 in a plant; b. contacting said plant with an effective amount of one or more rhizobia bacterium, arbuscular mycorrhiza fungi, or a combination thereof. In certain embodiments, said rhizobia bacterium is selected from the group consisting of: Sinorhizobium meliloti, Mesorhizobium loti, Sinorhizobium fredii, Rhizobium sp. IRBG74 and NGR234, Bradyrhizobium sp. In further embodiments, said arbuscular mycorrhiza fungi is selected from the group consisting of: Rhizophagus irregularis, Glomus mosseae, and Funneliformis mosseae. In another aspect, a modified plant, plant seed, plant part, or plant cell is provided, comprising a genomic modification that modulates the activity of LSH1 or LSH2, as compared to the activity of LSH1 or LSH2 in an otherwise identical plant, plant seed, plant part, or plant cell that lacks the modification. In certain embodiments, the modification is present in at least one allele of an endogenous LSH1 or LSH2 gene. For example, the genomic modification may be in an endogenous LSH1 or LSH2 gene encoding a protein having at least 70%, at least 75%, at least 80%, at least 85%, at least 90%, at least 95%, at least 96%, at least 97%, at least 98%, at least 99%, at least 99.5%, or 100% sequence identity to SEQ ID NO: 2 or 4. In some examples, the modification may be in a transcribable region of the LSH1 or LSH2 gene. The plant, plant seed, plant part, or plant cell may be heterozygous for the modification or homozygous for the modification. Modifications described herein may comprise a deletion, an insertion, a substitution, an inversion, a duplication, or a combination of any thereof. For example, the modification may comprise a deletion of at least 1, at least 3, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least at least 100, at least 125, or at least 150 consecutive nucleotides. A modified plant, plant seed, plant part, or plant cell provided herein may comprise a chromosomal sequence in the LSH1 or LSH2 gene that has at least 70%, at least 75%, at least 80%, at least 85%, at least 90%, at least 95%, at least 96%, at least 97%, at least 98%, at least 99%, at least 99.5%, or 100% sequence identity to SEQ ID NO: 1, 3, 5, 6, 7, or 8 in the regions outside of the deletion, the insertion, the substitution, the inversion, or the duplication. Methods are further provided for producing a plant comprising a modified LSH1 or LSH2 gene, the method comprising: a. introducing a modification into at least one target site in an endogenous LSH1 or LSH2 gene of a plant cell that modulates the activity of LSH1 or LSH2; b. identifying and selecting one or more corn plant cells of step a comprising said modification in said LSH1 or LSH2 gene; and c. regenerating at least a first plant from said one or more cells selected in step b or a descendent thereof comprising said modification. In another aspect, the present disclosure provides a recombinant DNA molecule comprising a DNA sequence selected from the group consisting of: a) a sequence with at least 85 percent sequence identity to any of SEQ ID NOs: 84-93; b) a sequence comprising any of SEQ ID NOs: 84-93; and c) a fragment of any of SEQ ID NOs: 84-93, wherein the fragment has gene-regulatory activity; wherein said sequence is operably linked to a heterologous transcribable DNA molecule. A recombinant DNA molecule as described herein may comprise a sequence having at least 90 percent sequence identity to the DNA sequence of any of SEQ ID NOs: 84-93, or a sequence having at least 95 percent sequence identity to the DNA sequence of any of SEQ ID NOs: 84-93, or a sequence comprising the DNA sequence of any of SEQ ID NOs: 84-93. Recombinant DNA molecules provided by the instant disclosure may comprise a heterologous transcribable DNA molecule comprising a gene of agronomic interest. Further provided are transgenic plant cells comprising the recombinant DNA molecule disclosed herein, which may be monocotyledonous plant cells or dicotyledonous plant cells. Transgenic plants, parts thereof, progeny plants, and transgenic seeds comprising the recombinant DNA molecules disclosed herein are further provided. The present disclosure further provides methods of producing a commodity product comprising obtaining a transgenic plant or part thereof according to the instant disclosure and producing the commodity product therefrom, including methods for producing commodity products such as protein concentrate, protein isolate, grain, starch, seeds, meal, flour, biomass, or seed oil. Further provided are methods of expressing a transcribable DNA molecule comprising obtaining a transgenic plant as described herein and cultivating the plant, wherein the transcribable DNA is expressed.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1: LSH1 and LSH2 are upregulated during early nodule organogenesis downstream of NIN. (A) Heatmap shows selected genes induced during lateral root and nodule development. Fold changes compared to controls are depicted in log 2 scale with the significance threshold of p-value<0.05. (B) Expression profiling on root segments treated with 100 nM 6-Benzylaminopurine (BAP) for 24 h by qRT-PCR normalized to HH3. Statistical comparisons between mock (white bars) and BAP (black bars). Values are the mean ΔCt values of 3 biological replicates for LSH1 and 2 for LSH2±SEM (Student's t-test; asterisks indicate statistical significance *, P<0.05; **, P<0.01, ***, P<0.001). (C) Expression patterns of LSH1 and LSH2 visualized by GUS staining. Rhizobial expressed LacZ is also stained. Ruthenium Red demarks cell walls. Asterisks indicate expression in vascular bundles and arrows in the meristem. Scale bars: 500 μm.

FIG. 2: LSH1/LSH2 are required for nodule development and N-fixation. (A-B) Images of WT, lsh1-2, lsh2-1 and lsh1-1/lsh2-1 dissected flower keels (A) and stipules (B). Scale bars: 500 μm. (C) Whole mount images of WT, lsh1-1, lsh2-1 and lsh1-1/lsh2-1 nodules 28 days post S. meliloti inoculation. GUS staining (blue) indicates the expression of the bacterial pNifH promoter. Scale bars: 500 μm. (D) Distribution of different nodule morphologies depicted as percentage of total nodule number per plant in WT and lsh1-1 (n=15), lsh2-1 (n=14), and lsh1-1/lsh2-1 (n=13). (E) Sections of 28-d old nodules in WT, lsh1-1 and lsh1-1/lsh2-1, pNifH:GUS expression (blue), Ruthenium Red stained cell walls. Scale bars: 500 μm.

FIG. 3. LSH genes are required for the development of nodule primordia that can support bacterial colonization. (A) Images of WT and lsh1/lsh2 nodule primordia at different developmental stages, first initial divisions (left), multilayered (middle) and emerged primordia (right) observed 7 d post spray inoculation with rhizobial bacteria expressing LacZ (blue stain). Black arrowheads indicate infection threads that are restricted in their progression into the inner root tissue layers. Squares relate to the legend in FIG. 3B. Scale bars: 500 μm. (B) Distribution of bacterial colonization phenotypes observed in WT, lsh1, lsh2 and lsh1/lsh2 at 7 dpi depicted as percentage of the total primordia number per plant, WT (n=30), lsh1, lsh2 (n=33), lsh1/lsh2 (n=32). (C) Optical sections of WT and lsh1/lsh2 roots 24 and 72 h post spot-inoculation with S. meliloti (n>30 per genotype and timepoint). At 72 hpi, Sm2011-mCherry bacteria in red, cell walls in white (fluorescent brightener) and EdU-labelled nuclei indicating DNA replication in green. White arrowheads indicate periclinal cell divisions. Scale bars: 50 μm.

FIG. 4. LSH1 and LSH2 are required for the upregulation of nodule organ identity genes and the recruitment of shoot-expressed genes during nodule organogenesis. (A) Heatmaps of all differentially expressed genes (DEGs) in response to S. meliloti spot inoculation in WT and lsh1 and lsh1/lsh2 at 24 and 72 hpi. Expression levels are depicted as log₂ fold changes (log₂ fold changes ≥+/−1, p-value<0.05). To compare the overall transcriptional response to S. meliloti spot inoculation between WT and the mutants, all DEGs were sorted from the highest positive to the highest negative log₂ fold change value. Absolute numbers indicate the number of DEGs in each genotype. Percentages indicate the proportion of DEGs in WT that are not expressed in the mutants and therefore dependent on LSH1 and LSH1/2. (B) Heatmaps showing expression levels of selected functional groups of DEGs in WT, lsh1 and lsh1/lsh2 root sections at 24 and 72 hpi and in response to combined ectopic expression of LSH1/LSH2 (pUBI:LSH1/LSH2) compared to empty vector in 3-week old WT (jemalong) hairy roots. Fold changes compared to controls are depicted in log₂ scale with the significance threshold of p-value<0.05. Green dots indicate that regulatory regions of these genes were bound by ectopically expressed LSH1-GFP in >50% of the 4 biological replicates at q-value<0.05.

FIG. 5. LSH1/LSH2 partly function through the cortical activation of NF-YA1. (A) Expression pattern of NF-YA1 in WT and lsh1/lsh2 visualized by GUS staining (blue) in whole mount images (left) and nodule sections (right). Rhizobial expressed LacZ is stained magenta. Ruthenium Red demarks cell walls in sections. Black asterisks indicate vascular expression restricted at the nodule base. Scale bars: 500 μm. (B) Heatmaps of all differentially expressed genes in response to S. meliloti spot inoculation in WT and in nf-ya1 at 24 and 72 hpi. Expression levels are depicted as log₂ fold changes (log₂ fold changes≥+/−1, p-value<0.05). Comparison of DEGs as described in FIG. 4A. Percentages indicate the proportion of DEGs in WT that are not expressed in the mutant and therefore dependent on NF-YA1. (C). Comparisons of all DEGs dependent on lsh1 (light purple), lsh1/lsh2 (dark purple) and nf-ya1 (green) up and down regulated at 24 hpi and 72 hpi. Genes with log₂ fold changes of ≥+/−1, p-value<0.05 were included in this analysis. (D) Heatmap of selected functional groups of DEGs in WT, lsh1, lsh1/lsh2 and nf-ya1 at 24 and 72 hours post S. meliloti spot inoculation and in response to combined ectopic expression of LSH1/LSH2 (pUBI:LSH1/LSH2) or NF-YA1 (pLjUBI:NF-YA1) compared to empty vector control in 3-week old WT (jemalong) hairy roots under non-symbiotic conditions. Fold changes compared to controls are depicted in log 2 scale with the significance threshold of p-value<0.05. (E) Whole mount images of nodules on hairy roots of lsh1/lsh2 plants transformed with empty vector control, pLSH1:LSH1, pLjUBI:NF-YA1, and combined pLSH1:NF-YA1/pLSH2:NF-YA1 at 28 dpi with S. meliloti expressing pNifH:GUS. GUS staining (blue) indicates the expression of the bacterial pNifH promoter. Scale bars: 500 μm.

FIG. 6. LSH1/LSH2 promote the expression of and act together with NOOT1/NOOT2 in the same regulatory pathways. (A) Expression patterns of NOOT1 and NOOT2 in WT and lsh1/lsh2 nodules, visualized by GUS staining (blue) in whole mount images (left) and nodule sections (right). Rhizobial expressed LacZ is stained magenta. Ruthenium Red demarks cell walls in sections. Black asterisks indicate vascular expression at the nodule base. Scale bars: 500 μm. (B). Heatmaps of all DEGs in WT, lsh1, lsh1/lsh2 and noot1/noot2 at 24 and 72 hpi. Expression levels are depicted as log₂ fold changes (log₂ fold changes ≥+/−1, p-value<0.05). Comparison as described in FIG. 4A. Percentages indicate the proportion of DEGs in WT that are not differentially expressed in the mutants and therefore dependent on LSH1, LSH1/2 and NOOT1/2. (C) Comparisons of all DEGs dependent on lsh1 (light purple), lsh1/lsh2 (dark purple) and noot1/noot2 (orange) up and down regulated at 24 hpi and 72 hpi. Genes with log₂ fold changes of ≥+/−1, p-value<0.05 were included in this analysis.

FIG. 7. LSH1/LSH2 and NOOT1/NOOT2 function synergistically to confer nodule organ identity. (A) Whole mount images of WT, lsh1, noot1/noot2 and lsh1/noot1 nodules at 21 days post S. meliloti inoculation. GUS staining (blue) indicates the expression of the bacterial pNifH. Scale bars: 500 μm. (B) Distribution of different nodule morphologies and N-fixation (pnifH:GUS staining) at 21 dpi depicted as percentage of the total nodule number per plant, WT (n=37), noot1/noot2 (n=44), lsh1 (n=33), lsh1/noot1 (n=35), lsh1/lsh2 (n=40). (C) Optical sections of WT, noot1/noot2 and lsh1/noot1 root sections 72 h post rhizobial spot-inoculation (n>15 per genotype). Sm2011-mCherry bacteria in red, cell walls in white (fluorescent brightener) and EdU-labelled nuclei indicating DNA replication in green. White arrowheads indicate periclinal cell divisions. Scale bars: 50 μm).

FIG. 8. NF-YA1 and LSH1/2 have in part overlapping functions. (A) Distribution of nodule morphologies/types categorised in “white”, “blue—partially pnifH:GUS expressing”, “blue—pnifH:GUS expressing” per plant in percentage for WT (n=12) and nf-ya1-1 (n=15) at 28 days post S. meliloti spray inoculation. Box plots show median (thick line), second to third quartiles (box), minimum and maximum ranges (lines), and outliers (single points). Student's t-tests showed that the distribution of the different nodule types is dependent on genotype; Asterisks indicated statistical significance *, P<0.05; **, P<0.01, ***, P<0.001). (B) Distribution of total nodule number per gram (g) root fresh weight of WT and nf-ya1-1 plants grown in terragreen:sand at 28 days post inoculation with S. meliloti. Asterisks indicated statistical significance *, P<0.05; **, P<0.01, ***, P<0.001, (Student's t-test). (C) Ectopic expression of NF-YA1 partially rescues lsh1-1 lsh2-1 nodule phenotype. Distribution of “white”, “partially blue” and “blue” nodules in absolute numbers and percentage of total nodule number per transformed hairy root system grown in terragreen:sand at 28 days post rhizobial spray inoculation.

FIG. 9. LSH1/2 and NOOT1/2 regulate overlapping pathways to confer nodule organ identity. (A) Distribution of nodule morphologies/types categorised in “white”, “blue—pnifH:GUS expressing”, “blue—pnifH:GUS expressing multilobed and/or fused”, “white multilobed and/or fused” and “root-like conversions” in percentage per plant for WT (n=37), noot1-1 noot2-1 (n=44), lsh1-1 (n=33), lsh1-1 noot1-1 (n=35) and lsh1-1 lsh2-1 (n=40) grown on plates at 21 days post S. meliloti spray inoculation. Box plots show median (thick line), second to third quartiles (box), minimum and maximum ranges (lines), and outliers (single points). One-way Kruskal-Wallis rank sum tests showed that the distribution of nodule types is dependent on genotype (KW=40.65, df=3, p=7.759e-09 (white), KW=47.00, df=3, p=3.468e-10 (blue—pnifH:GUS expressing), KW=26.181, df=3, p=8.742e-06 (blue—pnifH:GUS expressing multilobed and/or fused) and KW=41.64, df=3, p=4.783e-09 (white multilobed and/or fused). Asterisks indicate significantly different means for lsh1-1, lsh2-1 and lsh1-1 lsh2-1 compared with WT, Dunn Test (95% confidence). (B) Distribution of total nodule number per plant of WT, noot1-1 noot2-1, lsh-1, lsh1-1 noot1-1 and lsh1-1 lsh2-1 plants grown on plates 21 days post inoculation with S. meliloti. A one-way Kruskal-Wallis rank sum test showed that total nodule number per plant is dependent on genotype (KW=26.127, df=3, p<8.97e-06). Asterisk indicates a significantly different mean for lsh1-1 lsh2-1 compared with WT, Dunn Test (95% confidence). (C) Distribution of bacterial colonization phenotypes observed in WT, noot1-1 noot1-2, lsh1-1, lsh1-1 noot1-1 and lsh1 lsh2 plate-grown seedlings 7 dpi post S. meliloti inoculation, depicted as percentage of the total number of primordia per plant and categorised as “cortical infection in early multilayered primordia” (grey), “fully colonized emerged primordia” (black), “epidermal infection in early multilayered primordia” (white, hashed), “partially colonized emerged primordia” (grey hashed), and “uncolonized emerged primordia” (white), (n=23 for WT, n=28 for nf-ya1-1). (D) (E) Optical sections of additional noot1-1 noot2-1 and lsh1-1 noot1-1 root sections at 72 hours post spot-inoculated with S. meliloti. Infecting Sm2011-mCherry bacteria are labelled in red, fluorescent brightener (white) demarks cell walls and EdU-labelled nuclei (green) indicate DNA replication. Scale bars: 50 μm. (F) Rhizobial-induced genes that are dependent on NOOT1/2 function show a 99% overlap with LSH1/2-dependent rhizobial induced genes. Heatmaps of all differentially expressed genes in response to S. meliloti spot inoculation in WT, in the lsh1-1 single, the lsh1-1 lsh2-1 double mutant and the noot1-1 noot2-1 double mutant at 24 and 72 hpi. Expression levels are depicted as log₂ fold changes (log₂ fold changes ≥+/−1, p-value<0.05). To compare the overall transcriptional response to S. meliloti spot inoculation between WT and the mutants, all differentially expressed genes were sorted based on their log₂ fold changes. Absolute numbers indicate the number of DEGs in each genotype. Percentages indicate the proportion of differentially expressed genes in WT that are not differentially expressed in the mutants and therefore dependent on LSH1 and LSH1/2 and on NOOT1/2. (G) Comparisons of all differentially expressed genes dependent on lsh1-1 (light purple), lsh1-1 lsh1-2 (dark purple) and noot1-1 noot2-1 (orange) up and down regulated at 24 hip and 72 hpi. Genes with log₂ fold changes of ≥+/−1, p-value<0.05 were included in this analysis. (H) Comparison between all differentially expressed genes dependent on lsh1-1 and/or lsh1-1 lsh1-2 and noot1-1 noot2-1 and genes differentially expressed in response to ectopic expression of LSH1 and LSH2 combined or NOOT1 and NOOT2 combined under the constitutive Luba promoter under non-symbiotic conditions. Genes with log₂ fold changes of ≥+/−1, p-value<0.05 were included in this analysis.

FIG. 10. Amino acid sequences of LSH1 (SEQ ID NO: 2) and LSH2 (SEQ ID NO: 4). The ALOG domain (bolded) present in LSH1 extends from residue 52 to residue 179; and ALOG domain (bolded) present in LSH2 extends from residue 64 to residue 191.

FIG. 11. Ectopic LSH1 expression is sufficient to increase root length and diameter as compared to control plants.

FIG. 12. Ectopic LSH1 expression is sufficient to inhibit the progression and emergence of lateral root primordia.

FIG. 13. Overexpression of LSH1 in Medicago truncatula roots after rhizobia inoculation.

FIG. 14. Simultaneous overexpression of LSH1 and LSH2 in Medicago truncatula roots after rhizobia inoculation.

FIG. 15. MtLSH1 (Medtr1g069825) and MtLSH2 (Medtr7g097030) indicate the original Medicago LSH1 and LSH2 gene coding region. HvOptMtLSH1 and HvOptMtLSH2 indicate the barley codon optimized version of LSH1 and LSH2. pOsUBI3, pPvUBI2 and pZmUBI indicate the Oryza sativa (rice), Panicum virgatum (switchgrass), and Zea mays (maize) version of ubiquitin promoters respectively. The t35S represents Cauliflower Mosaic Virus (CaMV) 35S terminator; the tRbcS represents the ribulose-1,5-bisphosphate carboxylase (Rubisco) small subunit (rbcS) terminator. The nptII indicates the neomycin phosphotransferase selection system.

FIG. 16. Morphological comparison of NLSs collected from negative GUS control, MtLSH1, and MtLSH2 transformed roots in harvest 1. (A, D) The NLSs harvested from negative GUS control. (B, E) The NLSs harvested from pOsUbi::MtLSH1 transformed roots. (C, F) The NLSs harvested from pOsUbi::MtLSH2 transformed roots. (A-C) Sections are stained with Toluidine blue-O. (D-F) Maximum projection of Z-stack confocal images of NLSs. The pink color represents the mCherry signals from the transformation visual marker. The scale bar in each image indicates 200 μm. Vibratome section thickness is 100 μm.

FIG. 17. Morphological comparison of NLSs collected from negative GUS control and pOsUbi::MtLSH1 transformed roots after auxin treatments in harvest 2. (A, C, E) The NLSs harvested from negative GUS control. (B, D, F) The NLSs harvested from pOsUbi::MtLSH1 transformed roots. (A-B) Whole mount imaging of NLSs. (C-D) Sections are stained with Toluidine blue-O. (E-F) Maximum projection of Z-stack confocal images of NLSs. The pink color represents the mCherry signals from the transformation visual marker. (C-F) Vibratome section thickness is 100 μm. (A-F) The scale bar in each image indicates 200 μm.

FIG. 18. Morphological comparison of NLSs collected from negative GUS control and pOsUbi::HvOptMtLSH1 transformed roots in harvest 1. (A, C) The NLSs harvested from negative GUS control. (B, D) The NLSs harvested from pOsUbi::HvOptMtLSH1 transformed roots. (A-B) Sections are stained with Toluidine blue-O. (C-D) Maximum projection of Z-stack confocal images of NLSs. The pink color represents the mCherry signals from the transformation visual marker. (A-D) Vibratome section thickness is 100 μm. The scale bar in each image indicates 200 μm.

FIG. 19. Morphological comparison of NLSs collected from negative GUS control and Ubi::HvOptMtLSH1 transformed roots after auxin treatments in harvest 2. (A, C) The NLSs harvested from negative GUS control. (B, D) The NLSs harvested from pOsUbi::HvOptMtLSH1 transformed roots. (A-B) Sections are stained with Toluidine blue-O. (C-D) Maximum projection of Z-stack confocal images of NLSs. The pink color represents the mCherry signals from the transformation visual marker. (A-D) Vibratome section thickness is 100 μm. The scale bar in each image indicates 200 μm.

FIG. 20. Quantification of NLSs from the harvest before and after auxin treatments. (A) Quantification of the number of NLSs per plate collected from harvest 1. (B) Quantification of the frequency of NLSs per root collected from harvest 1. Frequency of NLSs=number of NLSs/length of the root (in centimeters).

FIG. 21. A domain tree showing protein sequences having SEQ ID NOs: 9-83 comprising a conserved ALOG domain region.

FIG. 22. Chip-Seq data showing a high confidence NIN-binding site upstream of LSH1.

FIG. 23. ChiP-Seq data showing putative direct targets of LSH1 CRE1, IPT1, RR19, CKX3, PIN1, STYLISH, PINOID, and NOOT1.

### BRIEF DESCRIPTION OF THE SEQUENCES

SEQ ID NO: 1 is a cDNA sequence encoding the Medicago truncatula LSH1 protein.

SEQ ID NO: 2 is the polypeptide sequence of the Medicago truncatula LSH1 protein, encoded by SEQ ID NO: 1. The ALOG domain extends from residue 52 to residue 179.

SEQ ID NO: 3 is a cDNA sequence encoding the Medicago truncatula LSH2 protein.

SEQ ID NO: 4 is the polypeptide sequence of the Medicago truncatula LSH2 protein, encoded by SEQ ID NO: 3. The ALOG domain extends from residue 64 to residue 191.

SEQ ID NO: 5 is a gDNA sequence encoding the Medicago truncatula LSH1 protein.

SEQ ID NO: 6 is a gDNA sequence encoding the Medicago truncatula LSH2 protein.

SEQ ID NO: 7 is a Hordeum vulgare codon-optimized nucleotide sequence encoding a LSH1 protein.

SEQ ID NO: 8 is a Hordeum vulgare codon-optimized nucleotide sequence encoding a LSH2 protein.

SEQ ID NOs: 9-84 are polypeptide sequences comprising a conserved ALOG domain region.

SEQ ID NO: 85 is the nucleotide sequence of the LSH1 promoter region including a putative NIN binding site at nucleotide 5,260.

SEQ ID NO: 86 is the nucleotide sequence of the LSH1 5′ UTR.

SEQ ID NO: 87 is the nucleotide sequence of the LSH1 intron.

SEQ ID NO: 88 is the nucleotide sequence of the LSH1 3′ UTR.

SEQ ID NO: 89 is the nucleotide sequence of the LSH1 downstream terminator region.

SEQ ID NO: 90 is the nucleotide sequence of the LSH2 promoter region including a putative NIN binding site at nucleotide 5000.

SEQ ID NO: 91 is the nucleotide sequence of the LSH2 5′ UTR.

SEQ ID NO: 92 is the nucleotide sequence of the LSH2 intron.

SEQ ID NO: 93 is the nucleotide sequence of the LSH2 3′ UTR.

SEQ ID NO: 94 is the nucleotide sequence of the LSH2 downstream terminator region.

### DETAILED DESCRIPTION OF THE INVENTION

Nitrogen-deficient or phosphate-deficient soils can result in low yield or plant death in crop plants, presenting a significant challenge globally. Symbiotic nitrogen-fixing bacteria can alleviate this challenge by improving plant biomass under low-nitrogen conditions. Legumes grow specialized root nodules to host beneficial nitrogen-fixing bacteria that provide plants with ammonia in exchange for carbon. These symbiotic nodules are distinct from lateral roots in morphology and function with nodules comprising of cells that accommodate nitrogen-fixing rhizobial bacteria endosymbiotically and provide favorable conditions for the biological nitrogen fixation process.

However, the gene regulatory process that leads to the formation of nodules that are distinct from lateral roots has not previously been understood. The inventors have therefore investigated the gene regulatory program that differentiates symbiotic root nodules from lateral roots in order to identify key regulators that establish nodule organ identity, i.e. an organ that can support the infection and accommodation of the nitrogen-fixing bacteria. The instant disclosure provides two members of the shoot-related Light Sensitive Short Hypocotyl (LSH) transcription factor family that were previously unknown and novel regulators of nodule organ identity, LSH1 and LSH2.

As demonstrated in the instant disclosure, LSH1 and LSH2 are required for the development of functional nodule primordia that can support the intercellular cortical infection, the intracellular colonization, and nitrogen-fixation by the bacteria. For example, the present disclosure demonstrates that LSH1 and LSH2 are required for the development of symbiotic root nodules that can host bacteria intracellularly and provide the environment for nitrogen fixation. LSH1/2 function includes, e.g., the cortex-specific promotion of the previously identified nodule organ identity regulators NF-YA1 and NOOT1/2 and therefore positions LSH1/2 as key integrators of nodule organ identity establishment and maintenance downstream of NIN.

Therefore, in some embodiments the present invention provides recombinant DNA molecules comprising a recombinant DNA molecule comprising a heterologous promoter operably linked to an LSH1 polynucleotide such as SEQ ID NO: 1, 5, or 7, or an LSH2 polynucleotide such as SEQ ID NO: 3, 6, or 8, or variants or fragments thereof. Plants heterologously expressing or overexpressing LSH1 or LSH2 proteins, for example, SEQ ID NO: 2, 4, or variants or fragments thereof, which promote symbiotic infections, are further provided. In further embodiments, plants heterologously expressing or overexpressing protein sequences comprising an ALOG domain such as SEQ ID NOs: 9-83, which promote symbiotic infections, are further provided.

### Symbiotic Bacteria

The present invention provides DNA molecules encoding proteins that when expressed in a plant may promote symbiotic bacterial infection, or express a transcribable polynucleotide molecule that promotes symbiotic bacterial infection and/or nitrogen fixation by symbiotic bacteria. “Symbiotic bacteria” as used herein, includes nitrogen-fixing bacteria. For example, rhizobia are bacteria found in soil that infect the roots of legumes and colonize root nodules which are involved in nitrogen utilization. As used herein, “rhizobia” refers to any diazotrophic bacteria that fix atmospheric nitrogen inside plants roots.

Plants comprising the recombinant DNA molecules described herein can be inoculated with nitrogen-fixing bacteria to produce improved agronomic effects including improved plant growth or increased yield or biomass under reduced nitrogen conditions. Symbiotic bacteria useful with the disclosed plants include, but are not limited to, Mesorhizobium loti, Sinorhizobium meliloti, Sinorhizobium fredii, Rhizobium sp. IRBG74 and NGR234, Bradyrhizobium sp. Thus, recombinant DNA molecules provided herein can be expressed in a plant in an amount effective to produce an increase in intercellular cortical infection, an increase in intracellular colonization by symbiotic bacteria, or an increase in nitrogen-fixation by symbiotic bacteria as compared to a wild-type or control plant. Additionally, recombinant DNA molecules provided herein can be expressed in a plant in an amount effective to result in rhizobial infection patterns; nodulation structures, such as cluster-like multi-lobed nodules; upregulation of nodule organ identity genes; recruit shoot-expressed genes during nodule organogenesis; a detectable amount of Rhizobial expressed LacZ; or promote cell proliferation, host cell differentiation, or endosymbiotic colonization in the primordium cell layers derived from the mid-cortex of the primary root.

compared to WT.; multiple vascular bundles branching out from the base and connecting to the primary root vasculature as compared to a wild-type or control plant. According to further embodiments, a modified plant is provided having an increase in intercellular cortical infection, an increase in intracellular colonization by symbiotic bacteria, or an increase in nitrogen-fixation by symbiotic bacteria by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a wild-type or control plant.

### DNA Molecules

As used herein, the term “DNA” or “DNA molecule” refers to a double-stranded DNA molecule of genomic or synthetic origin, i.e. a polymer of deoxyribonucleotide bases or a polynucleotide molecule, read from the 5′ (upstream) end to the 3′ (downstream) end. As used herein, the term “DNA sequence” refers to the nucleotide sequence of a DNA molecule. The nomenclature used herein corresponds to that of by Title 37 of the United States Code of Federal Regulations § 1.822, and set forth in the tables in WIPO Standard ST.25 (1998), Appendix 2, Tables 1 and 3.

As used herein, a “recombinant DNA molecule” is a DNA molecule comprising a combination of DNA molecules that would not naturally occur together without human intervention. For instance, a recombinant DNA molecule may be a DNA molecule that is comprised of at least two DNA molecules heterologous with respect to each other, a DNA molecule that comprises a DNA sequence that deviates from DNA sequences that exist in nature, a DNA molecule that comprises a synthetic DNA sequence or a DNA molecule that has been incorporated into a host cell's DNA by genetic transformation or gene editing.

As used herein, the term “isolated DNA molecule” refers to a DNA molecule at least partially separated from other molecules normally associated with it in its native or natural state. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

A polynucleotide or polypeptide provided herein may further include two or molecules which are heterologous with respect to one another. As used herein, the term “heterologous” refers to the combination of two or more polynucleotide molecules or two or more polypeptide molecules when such a combination is not normally found in nature. For example, the two molecules may be derived from different species and/or the two molecules may be derived from different genes, e.g. different genes from the same species or the same genes from different species. In some examples, a promoter is heterologous with respect to an operably linked transcribable polynucleotide molecule if such a combination is not normally found in nature, i.e. that transcribable polynucleotide molecule is not naturally occurring operably linked in combination with that promoter molecule.

Any number of methods well known to those skilled in the art can be used to isolate and manipulate a DNA molecule, or fragment thereof, disclosed in the present invention. For example, PCR (polymerase chain reaction) technology can be used to amplify a particular starting DNA molecule and/or to produce variants of the original molecule. DNA molecules, or fragment thereof, can also be obtained by other techniques such as by directly synthesizing the fragment by chemical means, as is commonly practiced by using an automated oligonucleotide synthesizer.

As used herein, the term “percent sequence identity,” “percent identity,” or “% sequence identity” refers to the percentage of identical nucleotides or amino acids in a linear polynucleotide or polypeptide sequence of a reference (e.g., “query”) sequence (or its complementary strand) as compared to a test (e.g., “subject”) sequence (or its complementary strand) when the two sequences are optimally aligned. An optimal sequence alignment is created by manually aligning two sequences, e.g. a reference sequence and another sequence, to maximize the number of nucleotide matches in the sequence alignment with appropriate internal nucleotide insertions, deletions, or gaps. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, CA), MEGAlign (DNAStar, Inc., 1228 S. Park St., Madison, Wis. 53715), and MUSCLE (version 3.6) (RC Edgar, Nucleic Acids Research (2004) 32(5):1792-1797) with default parameters. An “identity fraction” for aligned segments of a test sequence and a reference sequence is the number of identical components which are shared by the two aligned sequences divided by the total number of components in the reference sequence segment, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence. As used herein, the term “sequence identity” refers to the extent to which two optimally aligned polynucleotide sequences or two optimally aligned polypeptide sequences are identical. As used herein, the term “reference sequence,” for example, may refer to a sequence provided as the polynucleotide sequence of SEQ ID NO: SEQ ID NO: 1, 3, 5, 6, 7, or 8, or the polypeptide sequence of SEQ ID NO: 2 or 4. A “reference sequence” may also refer to a polypeptide sequence of SEQ ID NO: 9-83.

Thus, one embodiment of the invention is a recombinant DNA molecule comprising a sequence that when optimally aligned to a reference sequence, provided herein as the polynucleotide sequences of SEQ ID NO: 1, 3, 5, 6, 7, or 8 has at least about 70 percent identity, at least about 75 percent identity, at least about 80 percent identity, at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments such sequences may be defined as having the activity of the reference sequence, for example the activity of SEQ ID NO: 1, 3, 5, 6, 7, or 8.

Similarly, another embodiment of the invention is a polypeptide molecule comprising a sequence that when optimally aligned to a reference sequence, provided herein as the polypeptide sequences of SEQ ID NO: 2, 4, or 9-83, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments such sequences may be defined as having the activity of the reference sequence, for example the activity of SEQ ID NO: 2, 4, or 9-83.

Also provided are fragments of polynucleotide sequences provided herein, for example fragments of a polynucleotide sequence of SEQ ID NO: 1, 3, 5, 6, 7, or 8. In specific embodiments, fragments of a polynucleotide sequences are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule of SEQ ID NO: 1, 3, 5, 6, 7, or 8 or a sequence encoding SEQ ID NO: 2 or 4. Methods for producing such fragments from a starting molecule are well known in the art. Fragments, which can be functional fragments, of a polynucleotide sequence provided herein may comprise the activity or function of the base sequence.

Disclosed sequences may hybridize specifically to a target DNA sequence under stringent hybridization conditions. In certain embodiments, polynucleotides disclosed herein may hybridize under stringent hybridization conditions to a polynucleotide having the nucleotide sequence of SEQ ID NO: 1, 3, 5, 6, 7, or 8. Stringent hybridization conditions are known in the art and described in, for example, MR Green and J Sambrook, Molecular cloning: a laboratory manual, 4^{th} Edition, Cold Spring Harbor Laboratory Press, Cold Spring Harbor, N.Y. (2012). As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, two molecules exhibit “complete complementarity” if when aligned every nucleotide of the first molecule is complementary to every nucleotide of the second molecule. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Departures from complete complementarity are therefore permissible, as long as such departures do not completely preclude the capacity of the molecules to form a double-stranded structure.

Appropriate stringency conditions that promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

Recombinant polynucleotide sequences encoding fragments of polypeptide sequences provided herein are further envisioned, including polynucleotide sequences encoding fragments of a polypeptide sequence selected from SEQ ID NO: 2 or 4. In specific embodiments, fragments of a polypeptide are provided comprising at least about 50, at least about 75, at least about 95, at least about 100, at least about 125, at least about 150, at least about 175, at least about 200, at least about 221, or longer, of a polypeptide molecule of SEQ ID NO: 2 or 4. Methods for producing such fragments from a starting molecule are well known in the art. Fragments, which can be functional fragments, of a polynucleotide sequence provided herein may maintain the activity or function of the base sequence.

With respect to polypeptide sequences, the term “variant” as used herein refers to a second polypeptide sequence that is in composition similar, but not identical to, a first polypeptide sequence and yet the second polypeptide sequence still maintains the general functionality, i.e. same or similar activity, of the first polypeptide sequence. A variant may be a shorter or truncated version of the first polypeptide sequence and/or an altered version of the sequence of the first polypeptide sequence, such as one with different amino acid deletions, substitutions, and/or insertions. Variants having a percent identity to a sequence disclosed herein may have the same activity as the base sequence. For example, the transcribable polynucleotide molecule can encode a protein or variant of a protein or fragment of a protein that is functionally defined to maintain activity in transgenic host cells including plant cells, plant parts, explants, and whole plants.

Similarly, with respect to polynucleotide sequences, the term “variant” as used herein refers to a second polynucleotide sequence that is in composition similar, but not identical to, a first polynucleotide sequence and yet the second polynucleotide sequence still maintains the general functionality, i.e. same or similar activity, of the first polynucleotide sequence. A variant may be a shorter or truncated version of the first polynucleotide sequence and/or an altered version of the sequence of the first polynucleotide sequence, such as one with different nucleotide deletions, substitutions, and/or insertions. Variants having a percent identity to a sequence disclosed herein may have the same activity as the base sequence. For example, variant polynucleotides may encode the same or a similar protein sequence or have the same or similar gene regulatory activity as the base sequence.

As used herein, “modulation” of expression refers to the process of effecting either overexpression or suppression of a polynucleotide or a protein.

As used here, the term “overexpression” as used herein refers to an increased expression level of a polynucleotide or a protein in a plant, plant cell or plant tissue, compared to expression in a wild-type plant, cell or tissue, at any developmental or temporal stage for the gene. Overexpression can take place in plant cells normally lacking expression of polypeptides functionally equivalent or identical to the present polypeptides. Overexpression can also occur in plant cells where endogenous expression of the present polypeptides or functionally equivalent molecules normally occurs, but such normal expression is at a lower level. Overexpression thus results in a greater than normal production, or “overproduction” of the polypeptide in the plant, cell, or tissue.

### Constructs

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e., operably linked. As used herein, the term “vector” means any recombinant polynucleotide construct that may be used for the purpose of transformation, i.e., the introduction of heterologous DNA into a host cell. The term includes an expression cassette isolated from any of the aforementioned molecules.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell.

The constructs of the present invention may be provided, in one embodiment, as double Ti plasmid border DNA constructs that have the right border (RB or AGRtu.RB) and left border (LB or AGRtu.LB) regions of the Ti plasmid isolated from Agrobacterium tumefaciens comprising a T-DNA, that along with transfer molecules provided by the A. tumefaciens cells, permit the integration of the T-DNA into the genome of a plant cell (see, for example, U.S. Pat. No. 6,603,061). The constructs may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often A. tumefaciens ABI, C58, or LBA4404; however, other strains known to those skilled in the art of plant transformation can function in the present invention. For example, Agrobacterium rhizogenes ARqua1.

Methods are known in the art for assembling and introducing constructs into a cell in such a manner that the transcribable polynucleotide molecule is transcribed into a functional mRNA molecule that is translated and expressed as a protein product. For the practice of the present invention, conventional compositions and methods for preparing and using constructs and host cells are well known to one skilled in the art, see, for example, Molecular Cloning: A Laboratory Manual, 3^{rd} edition Volumes 1, 2, and 3 (2000) J. Sambrook, D. W. Russell, and N. Irwin, Cold Spring Harbor Laboratory Press. Methods for making recombinant vectors particularly suited to plant transformation include, without limitation, those described in U.S. Pat. Nos. 4,971,908; 4,940,835; 4,769,061; and 4,757,011 in their entirety. These types of vectors have also been reviewed in the scientific literature (see, for example, Rodriguez, et al., Vectors: A Survey of Molecular Cloning Vectors and Their Uses, Butterworths, Boston, (1988) and Glick, et al., Methods in Plant Molecular Biology and Biotechnology, CRC Press, Boca Raton, FL. (1993)). Typical vectors useful for expression of nucleic acids in higher plants are well known in the art and include vectors derived from the tumor-inducing (Ti) plasmid of Agrobacterium tumefaciens (Rogers, et al., Methods in Enzymology 153: 253-277 (1987)). Other recombinant vectors useful for plant transformation, including the pCaMVCN transfer control vector, have also been described in the scientific literature (see, for example, Fromm, et al., Proc. Natl. Acad. Sci. USA 82: 5824-5828 (1985)).

A construct provided herein may further comprise additional elements useful in regulating or modulating expression of a transcribable polynucleotide, including promoter, leader, enhancer, intron, and 3′ UTR sequences. A construct provided herein may further comprise one or more marker sequences for identification of the construct in plant cells, plant tissue, or plants.

### Transgenic Plants

Constructs, expression cassettes, and vectors comprising DNA molecules as disclosed herein can be constructed and introduced into a plant cell in accordance with transformation methods and techniques known in the art. For example, Agrobacterium-mediated transformation is described in U.S. Patent Application Publications 2009/0138985A1 (soybean), 2008/0280361A1 (soybean), 2009/0142837A1 (corn), 2008/0282432 (cotton), 2008/0256667 (cotton), 2003/0110531 (wheat), 2001/0042257 A1 (sugar beet), U.S. Pat. No. 5,750,871 (canola), U.S. Pat. No. 7,026,528 (wheat), and U.S. Pat. No. 6,365,807 (rice), and in Arencibia et al. (1998) Transgenic Res. 7:213-222 (sugarcane) all of which are incorporated herein by reference in their entirety. Transformed cells can be regenerated into transformed plants that express the polypeptides disclosed herein and demonstrate activity through bioassays as described herein as well as those known in the art. Plants can be derived from the plant cells by regeneration, seed, pollen, or meristem transformation techniques. Methods for transforming plants are known in the art.

The term “plant cell” or “plant” can include but is not limited to a dicotyledonous or monocotyledonous plant. In certain embodiments, plants provided herein are legumes, including, but not limited to, beans, soybeans, peas, chickpeas, peanuts, lentils, lupins, mesquite, carob, tamarind, alfalfa, and clover. Plants provided herein may also be non-legume plants.

The term “plant cell” or “plant” can also include but is not limited to an alfalfa, almond, Bambara groundnut, banana, barley, bean, black currant, broccoli, cabbage, blackberry, brassica, canola, carrot, cassava, castor, cauliflower, celery, chickpea, Chinese cabbage, citrus, coconut, coffee, corn (i.e., maize, such as sweet corn or field corn), clover, cotton, cowpea, a cucurbit, cucumber, Douglas fir, eggplant, eucalyptus, flax, forage legume, garlic, grape, hemp, hops, indigo, leek, legume, legume trees, lentil, lettuce, Loblolly pine, lotus, lupin, millets, melons, Medicago spp., nut, oat, olive, onion, ornamental, palm, pasture grass, pea, peanut, pepper, pigeon pea, pine, potato, poplar, pumpkin, pulses, Radiata pine, radish, rapeseed, raspberry, red currant, rice, rootstocks, rye, safflower, shrub, sorghum, Southern pine, soybean, spinach, squash, strawberry, sugar beet, sugarcane, sunflower, sweet gum, sweet potato, switchgrass, tea, tobacco, tomato, triticale, turf grass, walnut, watermelon, wheat, and yam plant cell or plant.

The term “plant cell” or “plant” can also include but is not limited to a cassava (e.g., manioc, yucca, Manihot esculenta), yam (e.g., Dioscorea rotundata, Dioscorea alata, Dioscorea trifida, Dioscorea sp.), sweet potato (e.g., Ipomoea batatas), taro (e.g., Colocasia esculenta), oca (e.g., Oxalis tuberosa), corn (e.g., maize, Zea mays), rice (e.g., indica rice, japonica rice, aromatic rice, glutinous rice, Oryza sativa, Oryza glaberrima), wild rice (e.g., Zizania spp., Porteresia spp.), barley (e.g., Hordeum vulgare), sorghum (e.g., Sorghum bicolor), millet (e.g., finger millet, fonio millet, foxtail millet, pearl millet, barnyard millets, Eleusine coracana, Panicum sumatrense, Panicum milaceum, Setaria italica, Pennisetum glaucum, Digitaria spp., Echinocloa spp.), teff (e.g., Eragrostis ten, oat (e.g., Avena sativa), triticale (e.g., X Triticosecale Wittmack, Triticosecale schlanstedtense Wittm., Triticosecale neoblaringhemii A. Camus, Triticosecale neoblaringhemii A. Camus), rye (e.g., Secale cereale, Secale cereanum), wheat (e.g., common wheat, spelt, durum, einkorn, emmer, kamut, Triticum aestivum, Triticum spelta, Triticum durum, Triticum urartu, Triticum monococcum, Triticum turanicum, Triticum spp.), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa, Trema levigata), apple (e.g., Malus domestica, Malus pumila, Pyrus malus), pear (e.g., Pyrus communis, Pyrus x bretschneideri, Pyrus pyrifolia, Pyrus sinkiangensis, Pyrus pashia, Pyrus spp.), plum (e.g., Mirabelle, greengage, damson, Prunus domestica, Prunus salicina, Prunus mume), apricot (e.g., Prunus armeniaca, Prunus brigantine, Prunus mandshurica), peach (e.g., Prunus persica), almond (e.g., Prunus dulcis, Prunus amygdalus), walnut (e.g., Persian walnut, English walnut, black walnut, Juglans regia, Juglans nigra, Juglans cinerea, Juglans californica), strawberry (e.g., Fragaria x ananassa, Fragaria chiloensis, Fragaria virginiana, Fragaria vesca), raspberry (e.g., European red raspberry, black raspberry, Rubus idaeus L., Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis, Rubus subgenus Eubatus sect. Moriferi & Ursini), red currant (e.g., white currant, Ribes rubrum), black currant (e.g., cassis, Ribes nigrum), gooseberry (e.g., Ribes uva-crispa, Ribes grossulari, Ribes hirtellum), melon (e.g., watermelon, winter melon, casabas, cantaloupe, honeydew, muskmelon, Citrullus lanatus, Benincasa hispida, Cucumis melo, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), bean (e.g., Phaseolus vulgaris, Phaseolus lunatus, Vigna angularis, Vigna radiate, Vigna mungo, Phaseolus coccineus, Vigna umbellate, Vigna acontifolia, Phaseolus acutifolius, Vicia faba, Vicia faba equine, Phaseolus spp., Vigna spp.), soybean (e.g., soy, soya bean, Glycine max, Glycine soja), pea (e.g., Pisum spp., Pisum sativum var. sativum, Pisum sativum var. arvense), pea (e.g., Pisum spp., Pisum sativum var. sativum, Pisum sativum var. arvense), chickpea (e.g., garbanzo, Bengal gram, Cicer arietinum), cowpea (e.g., Vigna unguiculata), pigeon pea (e.g., Arhar/Toor, cajan pea, Congo bean, gandules, Caganus cajan), lentil (e.g., Lens culinaris), Bambara groundnut (e.g., earth pea, Vigna subterranea), lupin (e.g., Lupinus spp.), pulses (e.g., minor pulses, Lablab purpureaus, Canavalia ensiformis, Canavalia gladiate, Psophocarpus tetragonolobus, Mucuna pruriens var. utilis, Pachyrhizus erosus), Medicago spp. (e.g., Medicago sativa, Medicago truncatula, Medicago arborea), Lotus spp. (e.g., Lotus japonicus), forage legumes (e.g., Leucaena spp., Albizia spp., Cyamopsis spp., Sesbania spp., Stylosanthes spp., Trifolium spp., Vicia spp.), indigo (e.g., Indigofera spp., Indigofera tinctoria, Indigofera suffruticosa, Indigofera articulata, Indigofera oblongifolia, Indigofera aspalthoides, Indigofera suffruticosa, Indigofera arrecta), legume trees (e.g., locust trees, Gleditsia spp., Robinia spp., Kentucky coffeetree, Gymnocladus dioicus, Acacia spp., Laburnum spp., Wisteria spp.), or hemp (e.g., cannabis, Cannabis sativa).

In certain embodiments, transgenic plants and transgenic plant parts regenerated from a transgenic plant cell are provided. In certain embodiments, the transgenic plants can be obtained from a transgenic seed, by cutting, snapping, grinding, or otherwise disassociating the part from the plant. In certain embodiments, the plant part can be a seed, a boll, a leaf, a flower, a stem, a root, or any portion thereof, or a non-regenerable portion of a transgenic plant part. As used in this context, a “non-regenerable” portion of a transgenic plant part is a portion that cannot be induced to form a whole plant or that cannot be induced to form a whole plant that is capable of sexual and/or asexual reproduction. In certain embodiments, a non-regenerable portion of a plant part is a portion of a transgenic seed, boll, leaf, flower, stem, or root.

The term “transformation” refers to the introduction of a DNA molecule into a recipient host. As used herein, the term “host” refers to bacteria, fungi, or plants, including any cells, tissues, organs, or progeny of the bacteria, fungi, or plants. Plant tissues and cells of particular interest include protoplasts, calli, roots, tubers, seeds, stems, leaves, seedlings, embryos, and pollen.

As used herein, the term “transformed” refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is inherited by subsequent progeny. A “transgenic” or “transformed” cell or organism may also include progeny of the cell or organism and progeny produced from a breeding program employing such a transgenic organism as a parent in a cross and exhibiting an altered phenotype resulting from the presence of a foreign DNA molecule. The introduced DNA molecule may also be transiently introduced into the recipient cell such that the introduced DNA molecule is not inherited by subsequent progeny. The term “transgenic” refers to a bacterium, fungus, or plant containing one or more heterologous DNA molecules.

There are many methods well known to those of skill in the art for introducing DNA molecules into plant cells. The process generally comprises the steps of selecting a suitable host cell, transforming the host cell with a vector, and obtaining the transformed host cell. Methods and materials for transforming plant cells by introducing a plant construct into a plant genome in the practice of this invention can include any of the well-known and demonstrated methods. Suitable methods can include, but are not limited to, bacterial infection (e.g., Agrobacterium), binary BAC vectors, direct delivery of DNA (e.g., by PEG-mediated transformation, desiccation/inhibition-mediated DNA uptake, electroporation, agitation with silicon carbide fibers, and acceleration of DNA coated particles), gene editing (e.g., CRISPR-Cas systems), among others.

Host cells may be any cell or organism, such as a plant cell, algal cell, algae, fungal cell, fungi, bacterial cell, or insect cell. In specific embodiments, the host cells and transformed cells may include cells from crop plants.

A transgenic plant subsequently may be regenerated from a transgenic plant cell of the invention. Using conventional breeding techniques or self-pollination, seed may be produced from this transgenic plant. Such seed, and the resulting progeny plant grown from such seed, will contain the recombinant DNA molecule of the present disclosure, and therefore will be transgenic.

Transgenic plants of the invention can be self-pollinated to provide seed for homozygous transgenic plants of the invention (homozygous for the recombinant DNA molecule) or crossed with non-transgenic plants or different transgenic plants to provide seed for heterozygous transgenic plants of the invention (heterozygous for the recombinant DNA molecule). Both such homozygous and heterozygous transgenic plants are referred to herein as “progeny plants.” Progeny plants are transgenic plants descended from the original transgenic plant and containing the recombinant DNA molecule of the invention. Seeds produced using a transgenic plant of the invention can be harvested and used to grow generations of transgenic plants, i.e., progeny plants of the invention, comprising the construct of this invention and expressing a gene of agronomic interest. Descriptions of breeding methods that are commonly used for different crops can be found in one of several reference books, see, e.g., Allard, Principles of Plant Breeding, John Wiley & Sons, NY, U. of CA, Davis, CA, 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

The transformed plants may be analyzed for the presence of the gene or genes of interest and the expression level and/or profile conferred by the regulatory elements of the invention. Those of skill in the art are aware of the numerous methods available for the analysis of transformed plants. For example, methods for plant analysis include, but are not limited to, Southern blots or northern blots, PCR-based approaches, biochemical analyses, phenotypic screening methods, field evaluations, and immunodiagnostic assays. The expression of a transcribable DNA molecule can be measured using TaqMan® (Applied Biosystems, Foster City, CA) reagents and methods as described by the manufacturer and PCR cycle times determined using the TaqMan® Testing Matrix. Alternatively, other methods and reagents for measuring expression of a transcribable DNA molecule are well known in the art. For example, the Invader® (Third Wave Technologies, Madison, WI) or SYBR Green (Thermo Fisher, A46012) reagents and methods as described by the manufacturer can be used to evaluate transgene expression.

Transgenic plants comprising recombinant DNA molecules as disclosed herein comprising a heterologous promoter operably linked to a polynucleotide segment encoding a light sensitive short hypocotyl protein may exhibit varying levels of expression of the polynucleotide segment over time. For example, a plant or part thereof maintained in a 12 hour/12 hour light/dark cycle may exhibit increased expression of the polynucleotide segment during the 12 hour light phase of the cycle. A plant or part thereof as described maintained in a 12 hour/12 hour light/dark cycle may exhibit increased expression of the polynucleotide segment during the first 6 hours, the first 5 hours, the first 4 hours, the first 3 hours, the first 2 hours, or the first hour of the light phase of the cycle. A plant or part thereof as described maintained in a 12 hour/12 hour light/dark cycle may exhibit increased expression of the polynucleotide segment during the first 1, 2, 3, 4, 5, 6, 7, 8, 9, 10, 11, 12, 13, 14, 15, 16, 17, 18, 19, 20, 21, 22, 23, or 24 hours of the cycle, or any combination thereof. The recitation of discrete values is understood to include ranges between each value.

The seeds of the plants of this invention can be harvested from fertile transgenic plants and be used to grow progeny generations of transformed plants of this invention including hybrid plant lines comprising the construct of this invention and expressing a gene of agronomic interest.

The present invention also provides for parts of the plants of the present invention. Plant parts, without limitation, include leaves, stems, roots, tubers, seeds, endosperm, ovule, and pollen. The invention also includes and provides transformed plant cells which comprise a nucleic acid molecule of the present invention.

The transgenic plant may pass along the transgenic polynucleotide molecule to its progeny. Progeny includes any regenerable plant part or seed comprising the transgene derived from an ancestor plant. The transgenic plant is preferably homozygous for the transformed polynucleotide molecule and transmits that sequence to all offspring as a result of sexual reproduction. Progeny may be grown from seeds produced by the transgenic plant. These additional plants may then be self-pollinated to generate a true breeding line of plants. Progeny from these plants are evaluated, among other things, for gene expression. The gene expression may be detected by several common methods such as western blotting, northern blotting, immuno-precipitation, and ELISA.

### Genome Modification

As an alternative to traditional transformation methods, a DNA molecule, such as a transgene, expression cassette(s), etc., may be inserted or integrated into a specific site or locus within the genome of a plant or plant cell via site-directed integration. Recombinant DNA construct(s) and molecule(s) of this disclosure may thus include a donor template sequence comprising at least one transgene, expression cassette, or other DNA sequence for insertion into the genome of the plant or plant cell. Such donor template for site-directed integration may further include one or two homology arms flanking an insertion sequence (i.e., the sequence, transgene, cassette, etc., to be inserted into the plant genome). The recombinant DNA construct(s) of this disclosure may further comprise an expression cassette(s) encoding a site-specific nuclease and/or any associated protein(s) to carry out site-directed integration. These nuclease expressing cassette(s) may be present in the same molecule or vector as the donor template (in cis) or on a separate molecule or vector (in trans). Several methods for site-directed integration are known in the art involving different proteins (or complexes of proteins and/or guide RNA) that cut the genomic DNA to produce a double strand break (DSB) or nick at a desired genomic site or locus. Briefly as understood in the art, during the process of repairing the DSB or nick introduced by the nuclease enzyme, the donor template DNA may become integrated into the genome at the site of the DSB or nick. The presence of the homology arm(s) in the donor template may promote the adoption and targeting of the insertion sequence into the plant genome during the repair process through homologous recombination, although an insertion event may occur through non-homologous end joining (NHEJ). Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome.

The present disclosure provides, in certain embodiments, plants, plant parts, plant cells, and seeds produced through genome modification using site-specific integration or genome editing. Genome editing can be used to make one or more edit(s) or mutation(s) at a desired target site in the genome of a plant, such as to change expression and/or activity of one or more genes, or to integrate an insertion sequence or transgene at a desired location in a plant genome. Any site or locus within the genome of a plant may potentially be chosen for making a genomic edit (or gene edit) or site-directed integration of a transgene, construct, or transcribable DNA sequence. As used herein, a “target site” for genome editing or site-directed integration refers to the location of a polynucleotide sequence within a plant genome that is bound and cleaved by a site-specific nuclease to introduce a double-stranded break (DSB) or single-stranded nick into the nucleic acid backbone of the polynucleotide sequence and/or its complementary DNA strand within the plant genome. A “target site” also refers to the location of a polynucleotide sequence within a plant genome that is bound and cleaved by any other site-specific nuclease that may not be guided by a non-coding RNA molecule, such as a zinc finger nuclease (ZFN), a transcription activator-like effector nuclease (TALEN), a meganuclease, etc., to introduce a DSB or single-stranded nick into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion, substitution, inversion, or duplication.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system), a TALE (transcription activator-like effector)-endonuclease (TALEN), a recombinase, or a transposase. As used herein, “editing” or “genome editing” refers to generating a targeted mutation, deletion, insertion, substitution, inversion, or duplication of at least 1, at least 2, at least 3, at least 4, at least 5, at least 6, at least 7, at least 8, at least 9, at least at least 15, at least 20, at least 25, at least 30, at least 35, at least 40, at least 45, at least 50, at least 75, at least 100, at least 250, at least 500, at least 1000, at least 2500, at least 5000, at least or at least 25,000 nucleotides of an endogenous plant genome nucleic acid sequence. As used herein, “editing” or “genome editing” may also encompass the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least or at least 25,000 nucleotides into the endogenous genome of a plant. An “edit” or “genomic edit” in the singular refers to one such targeted mutation, deletion, insertion, substitution, inversion, or duplication, whereas “edits” or “genomic edits” refers to two or more targeted mutation(s), deletion(s), insertion(s), substitution(s), inversion(s), and/or duplication(s), with each “edit” being introduced via a targeted genome editing technique.

### Genome Modified Plants

As used herein, “modified” in the context of a plant, plant seed, plant part, plant cell, and/or plant genome, refers to a plant, plant seed, plant part, plant cell, and/or plant genome comprising an engineered change in the expression level and/or endogenous sequence of one or more genes of interest relative to a wild-type or control plant, plant seed, plant part, plant cell, and/or plant genome. A modified plant refers to a plant having one or more differences including substitutions, insertions, deletions, inversions, duplications, or any desired combinations of such changes compared to a native polynucleotide or amino acid sequence. The term “modified” may further refer to a plant, plant seed, plant part, plant cell, and/or plant genome having one or more deletions affecting an endogenous LSH1 or LSH2 gene introduced through chemical mutagenesis, transposon insertion or excision, or any other known mutagenesis technique, or introduced through genome editing. In an aspect, a modified plant, plant seed, plant part, plant cell, and/or plant genome can comprise one or more transgenes. Therefore, a modified plant, plant seed, plant part, plant cell, and/or plant genome includes a mutated, edited and/or transgenic plant, plant seed, plant part, plant cell, and/or plant genome having a modified sequence of a LSH1 or LSH2 gene relative to a wild-type or control plant, plant seed, plant part, plant cell, and/or plant genome. Furthermore, the modification may increase, reduce, disrupt, or alter the activity of the protein encoded by a LSH1 or LSH2 gene as compared to the activity of the protein encoded by a LSH1 or LSH2 gene in an otherwise identical plant. As another example, the modified plant can overexpress LSH or increase LSH activity which can result in enlarged multilobed and fused nodules; nodule development and N-fixation; development of nodule primordia that can support bacterial colonization; upregulation of nodule organ identity genes; recruitment of shoot-expressed genes during nodule organogenesis; or formation of nodule like structures (NLSs) as compared to a wild-type or control plant, plant seed, plant part, plant cell, and/or plant genome.

Modified plants, plant parts, seeds, etc., may have been subjected to mutagenesis, genome editing or site-directed integration, genetic transformation, or a combination thereof. Such “modified” plants, plant seeds, plant parts, and plant cells include plants, plant seeds, plant parts, and plant cells that are offspring or derived from “modified” plants, plant seeds, plant parts, and plant cells that retain the molecular change (e.g., change in expression level and/or activity) to the LSH1 or LSH2 gene. A modified seed provided herein may give rise to a modified plant provided herein. A modified plant, plant seed, plant part, plant cell, or plant genome provided herein may comprise a recombinant DNA construct or vector or genome edit as provided herein. A “modified plant product” may be any product made from a modified plant, plant part, plant cell, or plant chromosome provided herein, or any portion or component thereof.

Modified plants may be further crossed to themselves or other plants to produce modified plant seeds and progeny. A modified plant may also be prepared by crossing a first plant comprising a DNA sequence or construct or an edit (e.g., a genomic deletion) with a second plant lacking the DNA sequence or construct or edit. For example, a DNA sequence or inversion may be introduced into a first plant line that is amenable to transformation or editing, which may then be crossed with a second plant line to introgress the DNA sequence or edit (e.g., deletion) into the second plant line. Progeny of these crosses can be further backcrossed into the desirable line multiple times, such as through 6 to 8 generations or back crosses, to produce a progeny plant with substantially the same genotype as the original parental line, but for the introduction of the DNA sequence or edit. A modified plant, plant cell, or seed provided herein may be a hybrid plant, plant cell, or seed. As used herein, a “hybrid” is created by crossing two plants from different varieties, lines, inbreds, or species, such that the progeny comprises genetic material from each parent. Skilled artisans recognize that higher order hybrids can be generated as well.

A modified plant, plant part, plant cell, or seed provided herein may be of an elite variety or an elite line. An “elite variety” or an “elite line” refers to a variety that has resulted from breeding and selection for superior agronomic performance.

As used herein, the term “control plant” (or likewise a “control” plant seed, plant part, plant cell, and/or plant genome) refers to a plant (or plant seed, plant part, plant cell, and/or plant genome) that is used for comparison to a modified plant (or modified plant seed, plant part, plant cell, and/or plant genome) and has the same or similar genetic background (e.g., same parental lines, hybrid cross, inbred line, testers, etc.) as the modified plant (or plant seed, plant part, plant cell, and/or plant genome), except for genome edit(s) (e.g., a deletion) affecting a ZmDA1 gene. For example, a control plant may be an inbred line that is the same as the inbred line used to make the modified plant, or a control plant may be the product of the same hybrid cross of inbred parental lines as the modified plant, except for the absence in the control plant of any transgenic events or genome edit(s) affecting an LSH1 or LSH2 gene. Similarly, an “unmodified control plant” refers to a plant that shares a substantially similar or essentially identical genetic background as a modified plant, but without the one or more engineered changes to the genome (e.g., mutation or edit) of the modified plant. For purposes of comparison to a modified plant, plant seed, plant part, plant cell, and/or plant genome, a “wild-type plant” (or likewise a “wild-type” plant seed, plant part, plant cell, and/or plant genome) refers to a non-transgenic and non-genome edited control plant, plant seed, plant part, plant cell, and/or plant genome. As used herein, a “control” plant, plant seed, plant part, plant cell, and/or plant genome may also be a plant, plant seed, plant part, plant cell, and/or plant genome having a similar (but not the same or identical) genetic background to a modified plant, plant seed, plant part, plant cell, and/or plant genome, if deemed sufficiently similar for comparison of the characteristics or traits to be analyzed.

As used herein, the term “activity” refers to the biological function of a gene or protein. A gene or a protein may provide one or more distinct functions. A reduction, disruption, or alteration in “activity” thus refers to a lowering, reduction, or elimination of one or more functions of a gene or a protein in a plant, plant cell, or plant tissue at one or more stage(s) of plant development, as compared to the activity of the gene or protein in a wild-type or control plant, cell, or tissue at the same stage(s) of plant development. Additionally, an increase in “activity” thus refers to an elevation of one or more functions of a gene or a protein in a plant, plant cell, or plant tissue at one or more stage(s) of plant development, as compared to the activity of the gene or protein in a wild-type or control plant, cell, or tissue at the same stage(s) of plant development. Similarly, “modulation” of activity refers to the process of effecting one or more functions of a gene or a protein in a plant, plant cell, or plant tissue at one or more stage(s) of plant development, as compared to the activity of the gene or protein in a wild-type or control plant, cell, or tissue at the same stage(s) of plant development.

According to some embodiments, a modified plant is provided having a genomic modification in an LSH1 or LSH2 gene that results in increased, reduced, disrupted, or altered activity of the protein encoded by the LSH1 or LSH2 gene in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to further embodiments, a modified plant is provided having a protein encoded by an LSH1 or LSH2 gene that results in increased, reduced, disrupted, or altered activity in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant. The recitation of ranges of values herein is merely intended to serve as a shorthand method of referring individually to each separate value falling within the range.

According to some embodiments, a modified plant is provided having an LSH1 or LSH2 mRNA level that is reduced or increased in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having an LSH1 or LSH2 mRNA expression level that is reduced or increased in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant. According to some embodiments, a modified plant is provided having a LSH1 or LSH2 protein expression level that is reduced or increased in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having an LSH1 or LSH2 protein expression level that is reduced or increased in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

### Commodity Products

The present invention provides a commodity product comprising DNA molecules according to the invention. As used herein, a “commodity product” refers to any composition or product which is comprised of material derived from a plant, seed, plant cell or plant part comprising a DNA molecule of the invention. Commodity products may be sold to consumers and may be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, flour, flakes, bran, fiber, milk, cheese, paper, cream, wine, and any other food for human consumption; and biomasses and fuel products. Viable commodity products include but are not limited to seeds and plant cells. Plants comprising a DNA molecule according to the invention can thus be used to manufacture any commodity product typically acquired from plants or parts thereof.

### Regulatory Elements

Regulatory elements such as promoters, leaders (also known as 5′ UTRs), enhancers, introns, and transcription termination regions (or 3′ UTRs) play an integral part in the overall expression of genes in living cells. The term “regulatory element,” as used herein, refers to a DNA molecule having gene-regulatory activity. The term “gene-regulatory activity,” as used herein, refers to the ability to affect the expression of an operably linked transcribable DNA molecule, for instance by affecting the transcription and/or translation of the operably linked transcribable DNA molecule. Regulatory elements, such as promoters, leaders, enhancers, introns and 3′ UTRs that function in plants are therefore useful for modifying plant phenotypes through genetic engineering.

The present disclosure provides regulatory elements including SEQ ID NOs: 84-93, or variants or fragments thereof, operably linked to a heterologous transcribable polynucleotide molecule. Regulatory elements may be characterized by their gene expression pattern, e.g., positive and/or negative effects such as constitutive expression or temporal, spatial, developmental, tissue, environmental, physiological, pathological, cell cycle, and/or chemically responsive expression, and any combination thereof, as well as by quantitative or qualitative indications. As used herein, a “gene expression pattern” is any pattern of transcription of an operably linked DNA molecule into a transcribed RNA molecule. The transcribed RNA molecule may be translated to produce a protein molecule or may provide an antisense or other regulatory RNA molecule, such as a double-stranded RNA (dsRNA), a transfer RNA (tRNA), a ribosomal RNA (rRNA), a microRNA (miRNA), and the like.

As used herein, the term “protein expression” is any pattern of translation of a transcribed RNA molecule into a protein molecule. Protein expression may be characterized by its temporal, spatial, developmental, or morphological qualities, as well as by quantitative or qualitative indications.

A promoter is useful as a regulatory element for modulating the expression of an operably linked transcribable DNA molecule. As used herein, the term “promoter” refers generally to a DNA molecule that is involved in recognition and binding of RNA polymerase II and other proteins, such as trans-acting transcription factors, to initiate transcription. A promoter may be initially isolated from the 5′ untranslated region (5′ UTR) of a genomic copy of a gene. Alternately, promoters may be synthetically produced or manipulated DNA molecules. Promoters may also be chimeric. Chimeric promoters are produced through the fusion of two or more heterologous DNA molecules. Promoters useful in practicing the present invention include promoter elements comprised within SEQ ID NOs: 84 and 89, or fragments or variants thereof. In specific embodiments of the invention, the claimed DNA molecules and any variants or derivatives thereof as described herein, are further defined as comprising promoter activity, i.e., are capable of acting as a promoter in a host cell, such as in a transgenic plant. In still further specific embodiments, a fragment may be defined as exhibiting promoter activity possessed by the starting promoter molecule from which it is derived, or a fragment may comprise a “minimal promoter” which provides a basal level of transcription and is comprised of a TATA box, other known transcription factor binding site motif, or equivalent DNA sequence for recognition and binding of the RNA polymerase II complex for initiation of transcription.

As used herein, the term “variant” refers to a second DNA molecule, such as a regulatory element, that is in composition similar, but not identical to, a first DNA molecule, and wherein the second DNA molecule still maintains the general functionality, i.e. the same or similar expression pattern, for instance through more or less equivalent transcriptional activity, of the first DNA molecule. A variant may be a shorter or truncated version of the first DNA molecule and/or an altered version of the sequence of the first DNA molecule, such as one with different restriction enzyme sites and/or internal deletions, substitutions, and/or insertions. A “variant” can also encompass a regulatory element having a nucleotide sequence comprising a substitution, deletion, and/or insertion of one or more nucleotides of a reference sequence, wherein the derivative regulatory element has more or less or equivalent transcriptional or translational activity than the corresponding parent regulatory molecule. Regulatory element “variants” will also encompass variants arising from mutations that naturally occur in bacterial and plant cell transformation. In the present invention, a polynucleotide sequence provided as SEQ ID NOs: 84-93 may be used to create variants that are in similar in composition, but not identical to, the DNA sequence of the original regulatory element, while still maintaining the general functionality, i.e., the same or similar expression pattern, of the original regulatory element. Production of such variants of the invention is well within the ordinary skill of the art in light of the disclosure and is encompassed within the scope of the invention.

Thus, the present disclosure provides variants of the regulatory elements disclosed herein, including SEQ ID NOs: 84-93. Variants provided sequences that, when optimally aligned to a reference sequence, provided herein as SEQ ID NOs: 84-93, have at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, at least about 99 percent identity, or at least about 100 percent identity to the reference sequence. Variants of SEQ ID NOs:84-93 provided herein may have the activity of the reference sequence from which they are derived.

Fragments of regulatory elements disclosed herein, including SEQ ID NO:84-93 are also provided. Fragments, which can be functional fragments, of regulatory elements may comprise gene-regulatory activity or function, and may be useful alone or in combination with other gene regulatory elements and fragments, such as in constructing chimeric promoters. In specific embodiments, fragments of a regulatory element are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule having gene-regulatory activity as disclosed herein. In certain embodiments, the fragments of any one of SEQ ID NOs: 84-93, having the activity of the full length sequence are provided. Methods for producing such fragments from a starting promoter molecule are well known in the art. The recitation of discrete values is understood to include ranges between each value.

As used herein, the term “intron” refers to a DNA molecule that may be isolated or identified from a gene and may be defined generally as a region spliced out during messenger RNA (mRNA) processing prior to translation. The present disclosure provide intron sequences including SEQ ID NO: 86 and 91, and variants and fragments thereof. Alternately, an intron may be a synthetically produced or manipulated DNA element. An intron may contain enhancer elements that effect the transcription of operably linked genes. An intron may be used as a regulatory element for modulating expression of an operably linked transcribable DNA molecule. A construct may comprise an intron, and the intron may or may not be heterologous with respect to the transcribable DNA molecule. Examples of introns in the art include the rice actin intron and the corn HSP70 intron.

As used herein, the terms “3′ transcription termination molecule,” “3′ untranslated region” or “3′ UTR” refer to a DNA molecule that is used during transcription to the untranslated region of the 3′ portion of an mRNA molecule. The present disclosure provide 3′ UTR sequences including SEQ ID NO: 87, 88, 92, and 93, and variants and fragments thereof. The 3′ untranslated region of an mRNA molecule may be generated by specific cleavage and 3′ polyadenylation, also known as a polyA tail. A 3′ UTR may be operably linked to and located downstream of a transcribable DNA molecule and may include a polyadenylation signal and other regulatory signals capable of affecting transcription, mRNA processing, or gene expression. PolyA tails are thought to function in mRNA stability and in initiation of translation. Examples of 3′ transcription termination molecules in the art are the nopaline synthase 3′ region; wheat hsp17 3′ region, pea rubisco small subunit 3′ region, cotton E6 3′ region, and the coixin 3′ UTR.

As used herein, the term “chimeric” refers to a single DNA molecule produced by fusing a first DNA molecule to a second DNA molecule, where neither the first nor the second DNA molecule would normally be found in that configuration, i.e. fused to the other. The chimeric DNA molecule is thus a new DNA molecule not otherwise normally found in nature. As used herein, the term “chimeric promoter” refers to a promoter produced through such manipulation of DNA molecules. A chimeric promoter may combine two or more DNA fragments; for example, the fusion of a promoter to an enhancer element. Thus, the design, construction, and use of chimeric promoters according to the methods disclosed herein for modulating the expression of operably linked transcribable DNA molecules are encompassed by the present invention.

Chimeric regulatory elements can be designed to comprise various constituent elements which may be operatively linked by various methods known in the art, such as restriction enzyme digestion and ligation, ligation independent cloning, modular assembly of PCR products during amplification, or direct chemical synthesis of the regulatory element, as well as other methods known in the art. The resulting various chimeric regulatory elements can be comprised of the same, or variants of the same, constituent elements but differ in the DNA sequence or DNA sequences that comprise the linking DNA sequence or sequences that allow the constituent parts to be operatively linked. A DNA sequence provided as SEQ ID NOs: 84-93 may provide a regulatory element reference sequence, wherein the constituent elements that comprise the reference sequence may be joined by methods known in the art and may comprise substitutions, deletions, and/or insertions of one or more nucleotides or mutations that naturally occur in bacterial and plant cell transformation.

Reference in this application to an “isolated DNA molecule”, or an equivalent term or phrase, is intended to mean that the DNA molecule is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

The efficacy of the modifications, duplications, or deletions described herein on the desired expression aspects of a particular transgene may be tested empirically in stable and transient plant assays, such as those described in the working examples herein, so as to validate the results, which may vary depending upon the changes made and the goal of the change in the starting DNA molecule.

### Transcribable DNA Molecules

As used herein, the term “transcribable DNA molecule” refers to any DNA molecule capable of being transcribed into a RNA molecule, including, but not limited to, those having protein coding sequences and those producing RNA molecules having sequences useful for gene suppression. The type of DNA molecule can include, but is not limited to, a DNA molecule from the same plant, a DNA molecule from another plant, a DNA molecule from a different organism, or a synthetic DNA molecule, such as a DNA molecule containing an antisense message of a gene, or a DNA molecule encoding an artificial, synthetic, or otherwise modified version of a transgene. Exemplary transcribable DNA molecules for incorporation into constructs of the invention include, e.g., DNA molecules or genes from a species other than the species into which the DNA molecule is incorporated or genes that originate from, or are present in, the same species, but are incorporated into recipient cells by genetic engineering methods rather than classical breeding techniques.

A regulatory element, such as any of SEQ ID NOs: 84-93 or variants or fragments thereof, may be operably linked to a transcribable DNA molecule that is heterologous with respect to the regulatory element. As used herein, the term “heterologous” refers to the combination of two or more DNA molecules when such a combination is not normally found in nature. For example, the two DNA molecules may be derived from different species and/or the two DNA molecules may be derived from different genes, e.g., different genes from the same species or the same genes from different species. A regulatory element is thus heterologous with respect to an operably linked transcribable DNA molecule if such a combination is not normally found in nature, i.e., the transcribable DNA molecule does not naturally occur operably linked to the regulatory element.

The transcribable DNA molecule may generally be any DNA molecule for which expression of a transcript is desired. Such expression of a transcript may result in translation of the resulting mRNA molecule, and thus protein expression. Alternatively, for example, a transcribable DNA molecule may be designed to ultimately cause decreased expression of a specific gene or protein. In one embodiment, this may be accomplished by using a transcribable DNA molecule that is oriented in the antisense direction. One of ordinary skill in the art is familiar with using such antisense technology. Any gene may be negatively regulated in this manner, and, in one embodiment, a transcribable DNA molecule may be designed for suppression of a specific gene through expression of a dsRNA, siRNA, or miRNA molecule.

Thus, one embodiment of the invention is a recombinant DNA molecule comprising a regulatory element of the invention, such as those provided as SEQ ID NOs: 84-93, operably linked to a heterologous transcribable DNA molecule so as to modulate transcription of the transcribable DNA molecule at a desired level or in a desired pattern when the construct is integrated in the genome of a transgenic plant cell. In one embodiment, the transcribable DNA molecule comprises a protein-coding region of a gene and in another embodiment the transcribable DNA molecule comprises an antisense region of a gene.

### Genes of Agronomic Interest

A transcribable DNA molecule may be a gene of agronomic interest. As used herein, the term “gene of agronomic interest” refers to a transcribable DNA molecule that, when expressed in a particular plant tissue, cell, or cell type, confers a desirable characteristic. The product of a gene of agronomic interest may act within the plant in order to cause an effect upon the plant morphology, physiology, growth, development, yield, grain composition, nutritional profile, disease or pest resistance, and/or environmental or chemical tolerance or may act as a pesticidal agent in the diet of a pest that feeds on the plant. In one embodiment of the invention, a regulatory element of the invention is incorporated into a construct such that the regulatory element is operably linked to a transcribable DNA molecule that is a gene of agronomic interest. In a transgenic plant containing such a construct, the expression of the gene of agronomic interest can confer a beneficial agronomic trait. A beneficial agronomic trait may include, for example, but is not limited to, herbicide tolerance, insect control, modified yield, disease resistance, pathogen resistance, modified plant growth and development, modified starch content, modified oil content, modified fatty acid content, modified protein content, modified fruit ripening, enhanced animal and human nutrition, biopolymer productions, environmental stress resistance, pharmaceutical peptides, improved processing qualities, improved flavor, hybrid seed production utility, improved fiber production, and desirable biofuel production.

Alternatively, a gene of agronomic interest can affect the above mentioned plant characteristics or phenotypes by encoding a RNA molecule that causes the targeted modulation of gene expression of an endogenous gene, for example by antisense (see, e.g. U.S. Pat. No. 5,107,065); inhibitory RNA (“RNAi,” including modulation of gene expression by miRNA, siRNA-, trans-acting siRNA-, and phased sRNA-mediated mechanisms, e.g., as described in published applications U.S. 2006/0200878 and U.S. 2008/0066206, and in U.S. patent application Ser. No. 11/974,469); or cosuppression-mediated mechanisms. The RNA could also be a catalytic RNA molecule (e.g., a ribozyme or a riboswitch; see, e.g., U.S. 2006/0200878) engineered to cleave a desired endogenous mRNA product. Methods are known in the art for constructing and introducing constructs into a cell in such a manner that the transcribable DNA molecule is transcribed into a molecule that is capable of causing gene suppression.

### Selectable Markers

Selectable marker transgenes may also be used with the regulatory elements of the invention. As used herein the term “selectable marker transgene” refers to any transcribable DNA molecule whose expression in a transgenic plant, tissue, or cell, or lack thereof, can be screened for or scored in some way. Selectable marker genes, and their associated selection and screening techniques, for use in the practice of the present disclosure are known in the art and include, but are not limited to, transcribable DNA molecules encoding β-glucuronidase (GUS), green fluorescent protein (GFP), proteins that confer antibiotic resistance, and proteins that confer herbicide tolerance.

When introducing elements of the present disclosure or the embodiment(s) thereof, the articles “a”, “an”, “the”, and “said” are intended to mean that there are one or more of the elements.

The term “and/or”, when used in a list of two or more items, means any one of the items, any combination of the items, or all of the items with which this term is associated.

The terms “comprising”, “including”, and “having” are intended to be inclusive and mean that there may be additional elements other than the listed elements. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

Having now generally described the invention, the same will be more readily understood through reference to the following examples which are provided by way of illustration, and are not intended to be limiting of the present invention, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1: LSH1 and LSH2 are Upregulated During Early Nodule Organogenesis Downstream of NIN

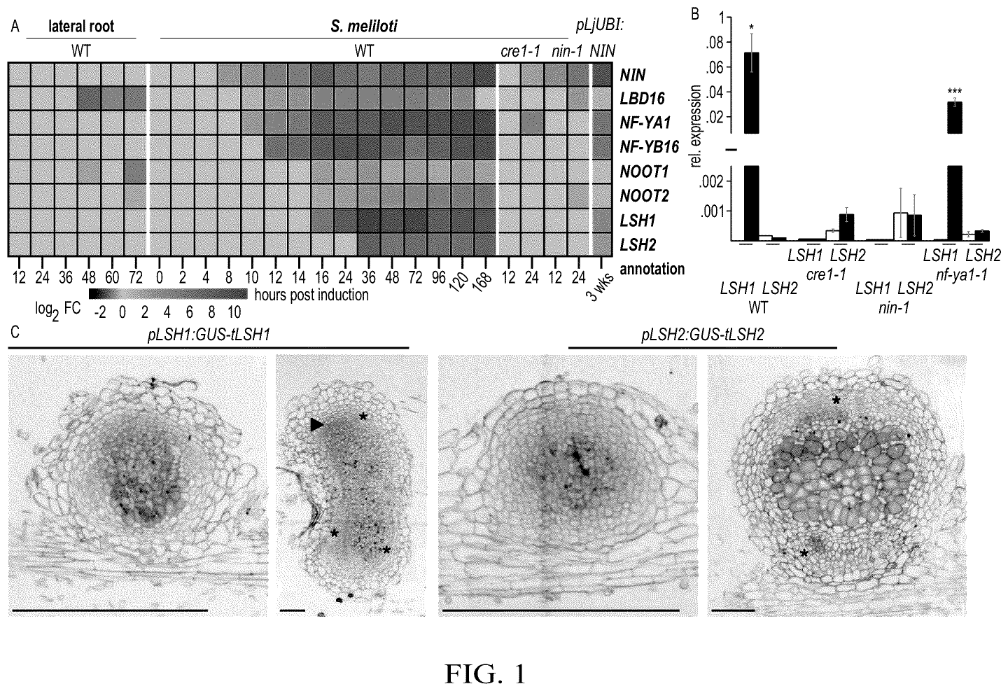

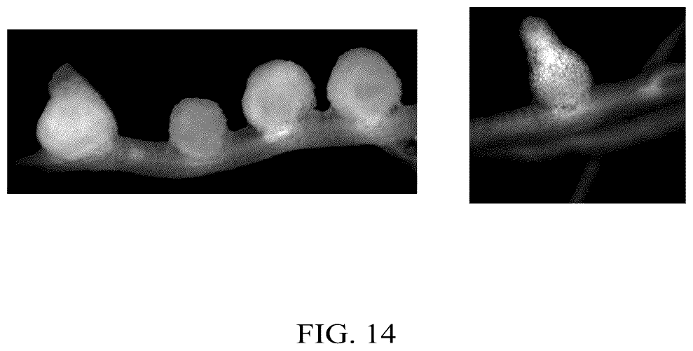

Although previous comparisons between lateral roots and nodules have revealed extensive overlap in morphology and transcription at their initiation stage, nodules are significantly differentiated from lateral roots as development progresses. To better understand the gene regulatory network that differentiates a nodule from a lateral root over the course of development, changes in gene expression were observed and correlated with the timepoints when the first morphological differences between lateral root and nodule primordia occur. This comparison identified a set of previously characterized nodule organ identity regulators besides LBD16, including NF-YA1, a previously identified putative downstream target of NIN, the NF-YA1-interacting subunit NF-YB16 and the transcriptional co-activators NOOT1 and NOOT2 to be upregulated at these timepoints in a nodule-specific manner. Importantly, two previously unknown transcriptional regulators with yet uncharacterized functions were identified in symbiotic nodulation that showed similar expression patterns (FIG. 1A). Both regulators contained an ALOG domain (FIG. 13) and showed high sequence similarity to members of the LIGHT SENSITIVE SHORT HYPOCOTYL (LSH) transcription factor family. Accordingly, these novel transcriptional regulators were given the designations “MtLSH1” and “MtLSH2,” referred to herein as LSH1 and LSH2. LSH1 is upregulated in roots from 16 hrs post rhizobial spot inoculation, while LSH2 is upregulated from 36 hrs. By contrast, neither LSH1 or LSH2 were differentially expressed during lateral root development, suggesting that LSH1 and LSH2 may be part of a developmental program that distinguishes nodules from lateral roots (FIG. 1A). The expression of LSH1 and LSH2 during rhizobial infection was dependent on CRE1 and NIN, and ectopic expression of NIN was sufficient to upregulate both genes (FIG. 1A). Furthermore, we identified a DNA-binding site of NIN in the exon of LSH1 using Chromatin-Immunoprecipitation using hairy roots expressing LjUBI:GFP-NIN under non-symbiotic conditions, suggesting that LSH1 but not LSH2 is a direct putative target of NIN. Furthermore, expression of LSH1 was induced by cytokinin treatment of M. truncatula roots in a CRE1- and NIN-dependent but NF-YA1-independent manner (FIG. 1B). Together, this identifies LSH1 and LSH2 as putative nodule organ identity regulators that are specifically recruited during early symbiotic nodule organogenesis in a cytokinin- and NIN-dependent manner.

To investigate the spatial expression patterns of both LSH genes promoter-GUS analysis of LSH1 and LSH2 was performed, which revealed that both genes are expressed in nodule primordia throughout development. In mature nodules, LSH1 is expressed in the apical meristem region and LSH2 is expressed in the infection and fixation zones, with both genes expressed in the peripheral nodule vasculature (FIG. 1C). Together, this suggests that LSH1 and LSH2 are upregulated in the root in response to symbiotic signaling and are expressed throughout nodule development in Medicago truncatula.

### Example 2: LSH Genes are Required for the Development of Nodules that can Support Nitrogen Fixation

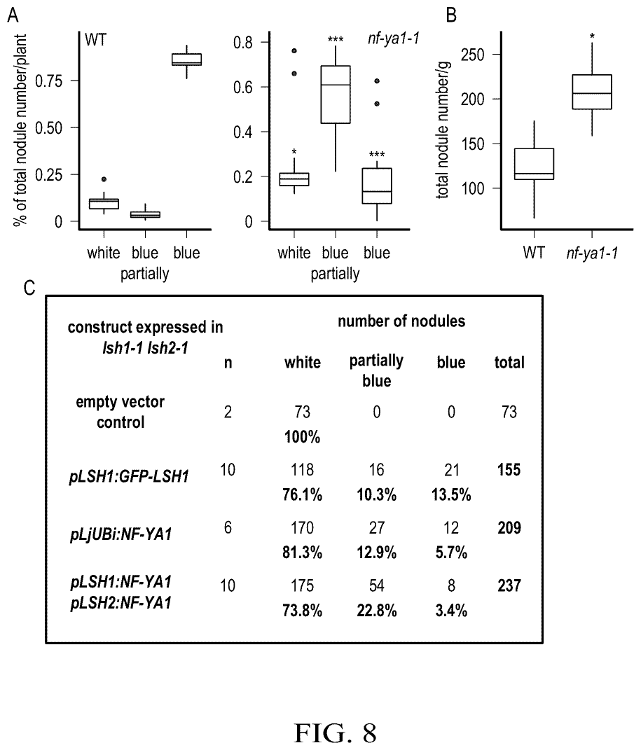

To further assess the role of the LSH1 and LSH2 genes during nodule organogenesis, loss of function mutants in LSH1 (lsh1-1, lsh1-2) and LSH2 (lsh2-1) were identified and the lsh1-1 lsh2-1 double mutant was generated. The lsh1 loss of function mutants but not the lsh2-1 mutant showed significantly altered shoot organ morphologies including changes in petal shape and number, and a reduction in stipule complexity (FIGS. 8B and E). No effects of LSH have been reported on root system architecture, and we also found that the root morphology was unaffected in lsh1/lsh2 seedlings and that LSH1/LSH2 are not positive regulators of lateral root development. In fact a slight increase in the number of lateral roots was observed in lsh1 and lsh1/lsh2 seedlings besides a slight reduction in primary root length, suggesting that in M. truncatula these genes are negative regulators of lateral root initiation.

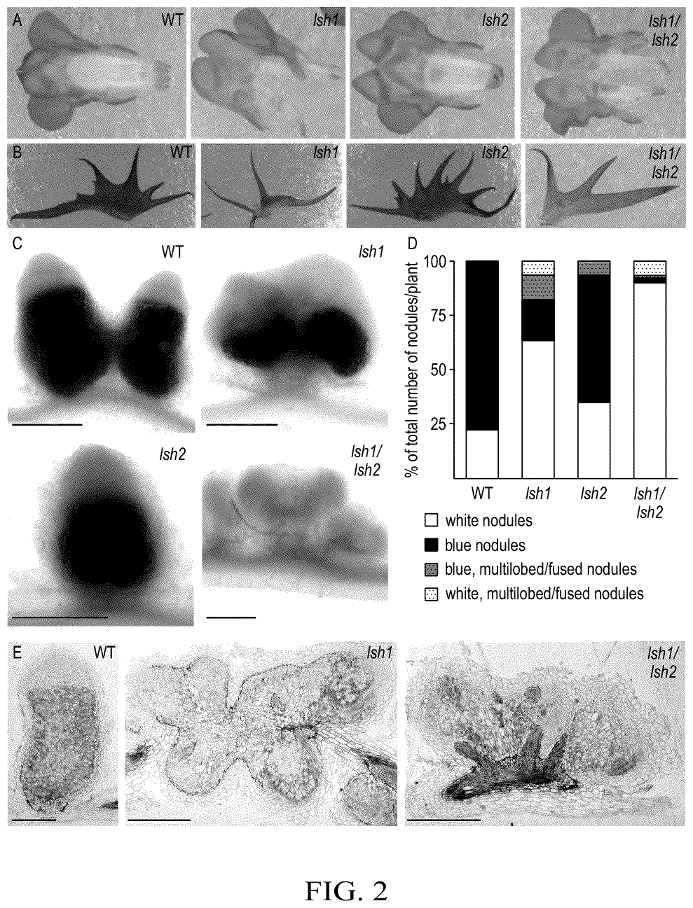

To assess the overall effect of loss of LSH1 and LSH2 on nodulation and nitrogen fixation, plants grown in terragreen:sand mix were inoculated with the Sinorhizobium meliloti strain 2011 expressing GUS under the bacterial pnifH promoter, a bacterial promoter associated with the expression and activity of nitrogenase used to approximate biological N-fixation in nodules. Using the pnifH:GUS reporter to distinguish blue, nitrogen (N)-fixing from white non-fixing nodules at 28 days post inoculation (dpi), a significant reduction in the ratio between blue and white nodules in the lsh1 mutants compared to wildtype was found, while the lsh2-1 mutant showed a ratio comparable to wildtype (or showed only a minor change in the ratio between white nodules and mature N-fixing nodules compared to wild type) (FIGS. 2C and D, FIGS. 8F and I). In the lsh1-1 lsh2-1 double mutant, white nodules were almost exclusively observed suggesting that nitrogen fixation was severely attenuated in this double mutant (FIGS. 2C and D). This was further confirmed in an acetylene reduction assay which showed a significant reduction in the nitrogenase activity of lsh1-1 nodules compared to WT and a complete abolishment of nitrogenase activity in the lsh1-1 lsh2-1 double mutant nodules at 21 dpi (FIG. 8H), confirming that the pnifH-GUS staining serves as a reliable approximation for nitrogen fixation in nodules. In addition, a significant increase of nodule number in the lsh1-1 lsh2-1 mutant was observed compared to wildtype and the single mutants, a phenomenon frequently observed in fix mutants (FIG. 8I). Furthermore, the nodule morphology was significantly altered in the lsh1 and the lsh1-1 lsh2-1 mutants compared to wildtype, with an increased number of enlarged multilobed and fused nodules observed in the lsh1 mutants and stunted, small and fused nodules observed in the lsh1-1 lsh2-1 mutant (FIGS. 2C-E and 8F and G).

### Example 3: LSH Genes are Required for the Development of Nodule Primordia that can Support Bacterial Colonization

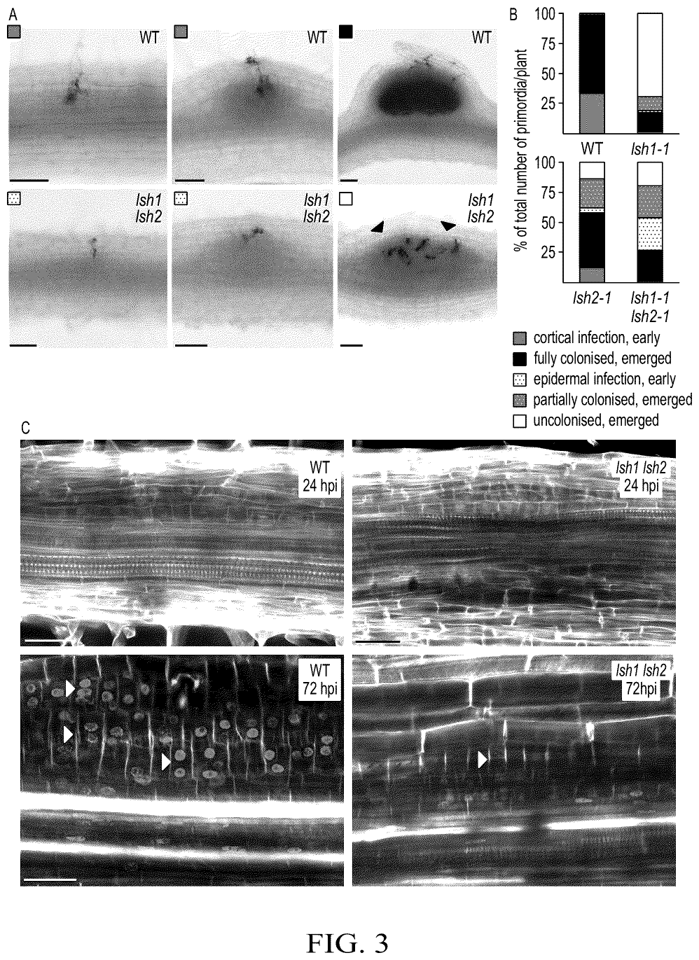

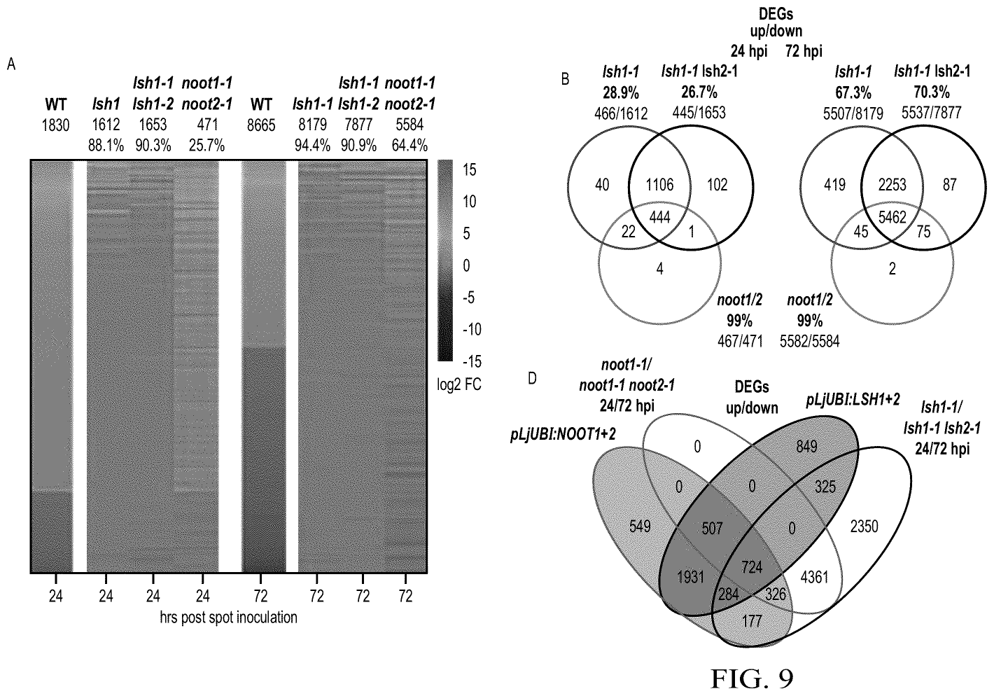

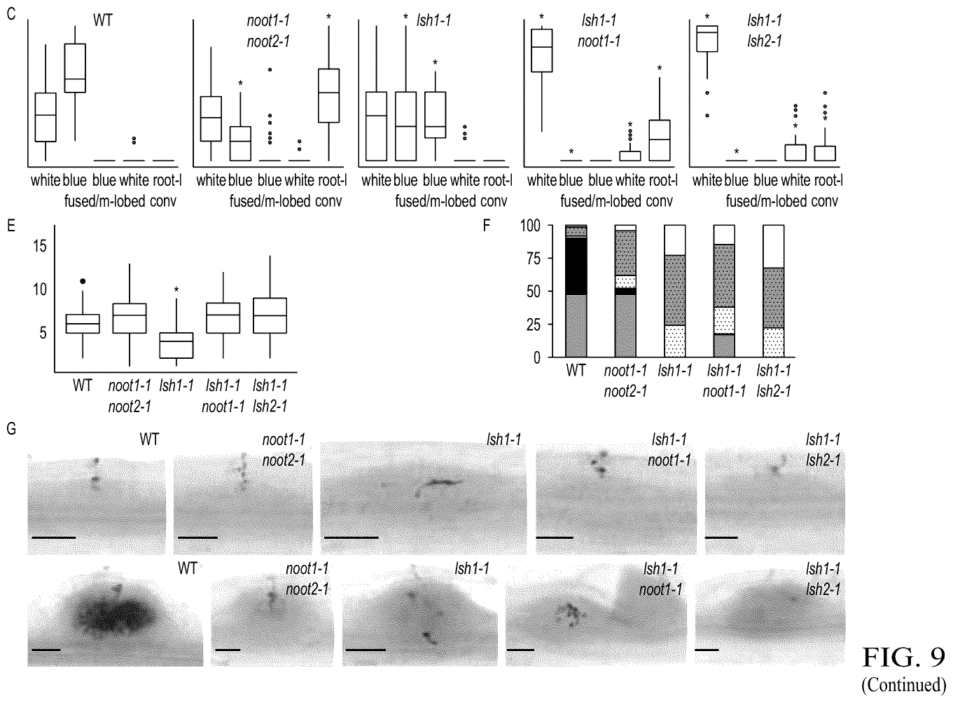

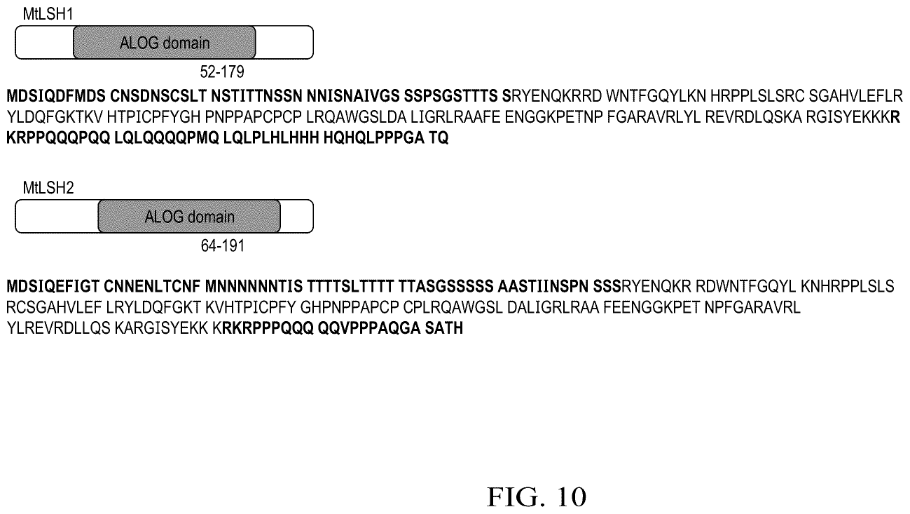

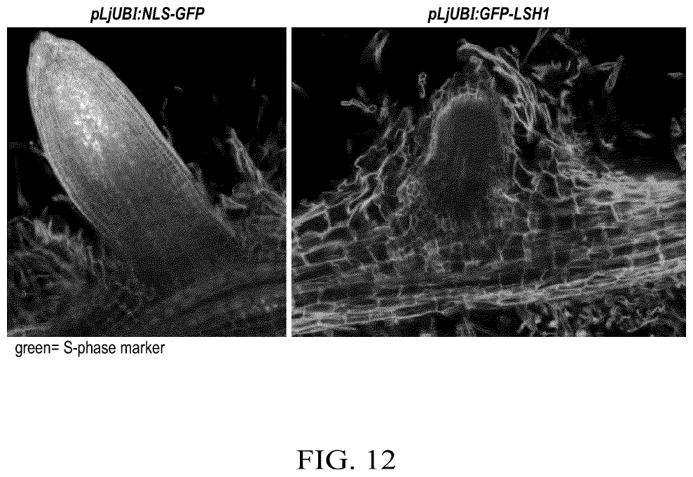

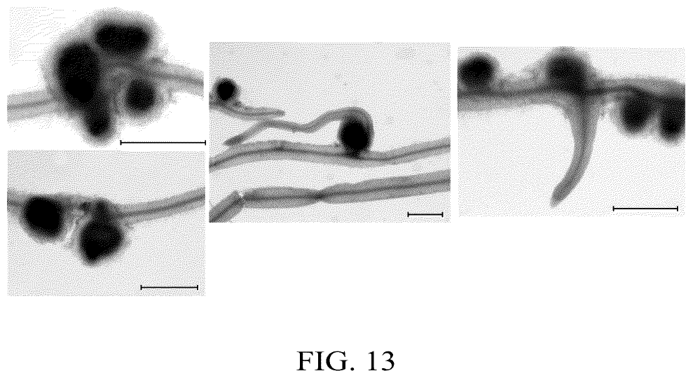

To further investigate the cause of this severe reduction in N-fixation, the progression of the rhizobial infection process and early nodule primordium development was investigated in plate-grown seedlings 7 days post inoculation with the S. meliloti strain 2011 lacZ. To this end, all rhizobial infection events were counted and assessed along the full length of the primary root of the seedlings and the analysis focused on the infection events that had either early cell divisions in the inner root tissue layers or developing nodule primordia associated with them. In the wildtype, it was observed that the progression of the rhizobial infection threads through the epidermis and cortex towards the dividing nodule primordium cells was temporally and spatially coordinated with the development of the nodule primordium, which started from a few cell divisions in the inner tissue layers and developed to a multilayered primordium. This coordinated progression of both processes results in early wildtype nodule primordia that are fully colonized at the timepoint of their emergence from the primary root (FIGS. 3A and B and FIG. 9). Similar to the wild type, it was observed that nodule primordia were initiating and developing in response to the successful establishment of the epidermal infection in the lsh1-1 and lsh1-1 lsh2-1 mutants. However, the progression of the rhizobial infection threads through the cortex and subsequent internal colonization of primordium cells was severely impaired in lsh1-1 and lsh1-1 lsh2-1 mutants (FIGS. 3A-B and FIG. 9). This resulted in a high proportion of lsh1-1 and lsh1-1 lsh2-1 mutant primordia that were only partially or in the most severe cases completely uncolonized at the time point of emergence from the primary root (FIGS. 3A-B and 12F and G). In addition, rhizobial bacteria propagating on the surface of the uncolonized or partially colonized nodule primordia was observed (FIGS. 3A and 12G). This suggests that unlike LBD16, LSH1 and LSH2 are not required for the initiation of the nodule primordium but that they play a major role in the infectability of the overlaying cortical tissue below the site of epidermal infection and in the habitability of the developing nodule primordium.

To study these early infection and colonization defects with increased temporal and spatial resolution, rhizobial spot inoculation was used combined with deep tissue imaging of the DNA synthesis marker 5-ethynyl-2-deoxyuridine (EdU) combined with either propidium iodide or fluorescent brightener as a cell wall marker. At 24 hrs post rhizobial spot inoculation (24 hpi), no obvious differences in the cell cycle activity and overall primordium development between WT and the lsh1-1 lsh2-1 double mutant were observed (FIG. 3C top panel). By contrast, a severe reduction in cell cycle activity in lsh1-1 lsh2-1 double mutant primordia compared to WT at 72 hpi (FIGS. 3C bottom panel and 9) was observed. This reduction in cell cycle activity was specific to the primordium cells that derived from the middle cortex of the primary root, while the cell cycle activity in the inner tissue layers at the base of the primordia was comparable between WT and the lsh1-1 lsh2-1 double mutant. More specifically, the cells in the cortical cell layers of WT primordia predominantly had been dividing in periclinal orientation at 72 hpi, adding cortical derived cell layers to the growing primordium. By contrast, these periclinal cell divisions were severely reduced or completely abolished in the lsh1-1 lsh2-1 double mutant primordia at 72 hpi (FIGS. 3C bottom panel and 9). Together, this suggests that LSH1 and LSH2 are required to specifically promote periclinal cell divisions in the distal, cortical derived part of the developing nodule primordium and that these cortical divisions appear to be causally linked to the successful progression of the infection thread through the overlaying cortical cell layers.

### Example 4: LSH1 and LSH2 are Required for the Upregulation of Nodule Organ Identity Genes and the Recruitment of Shoot-Related Genes into Nodule Organogenesis

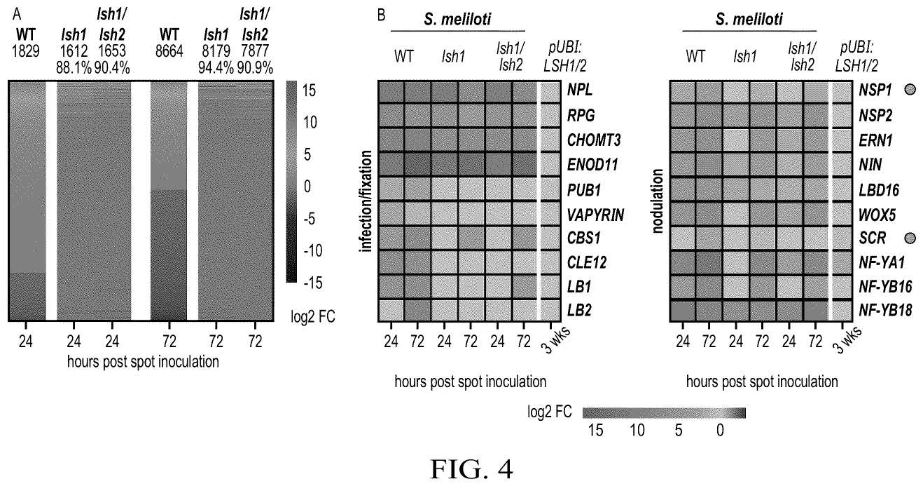

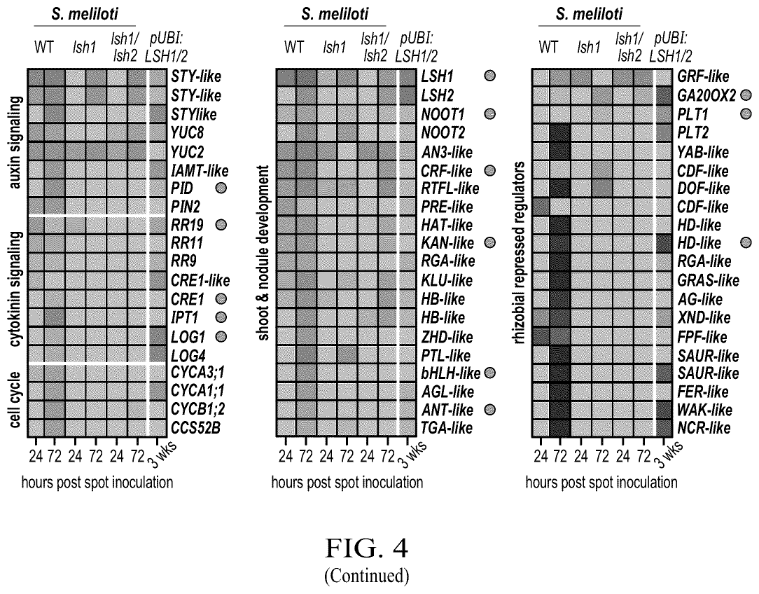

In order to better understand the regulatory function of the transcription factors LSH1 and LSH2 during rhizobial infection and nodule organogenesis, RNA-Seq was performed on rhizobial spot inoculated root sections of the lsh1-1 single, the lsh1-1 lsh2-1 double mutant and the corresponding wildtype (ecotype R108) at 24 and 72 hpi. In addition, hairy roots expressing pLjUBI:LSH1 and pLjUBI:LSH2 combined were generated and RNA-Seq was performed on hairy roots under nonsymbiotic conditions. Hairy roots expressing pLjUBI:GFP-LSH1 and pLjUBI-NLS-GFP were generated as control and Chromatin-Immunoprecipitation was performed followed by next-generation sequencing (chromatin immunoprecipitation sequencing; ChiP-Seq) under non-symbiotic conditions. Consistent with an early role in primordium formation, lsh1-1 and lsh1-1 lsh2-1 mutants showed severe reductions in nodule-associated gene expression compared to the wildtype, with over 90% of rhizobial-responsive genes in WT being dependent on LSH1/LSH2 at 24 hpi and 72 hpi (FIG. 4A). Marker genes for symbiosis signaling, such as EARLY NODULIN 11 (ENOD11) and NIN, were still expressed in lsh1/lsh2, as were genes associated with early infection such as Nodule Pectate Lyase (NPL) and Rhizobium-directed Polar Growth (RPG) (FIG. 4B). In contrast, genes associated with infection progression and N-fixation such as VAPYRIN (VYP) and LEGHEMOGLOBINs (LB1/LB2) were not upregulated in the lsh1/lsh2 mutant (FIG. 4B). Of the genes known to be associated with the initiation of the nodule primordium, LBD16 expression was not significantly affected by LSH mutation, whereas the known nodule regulators NF-YA1 and NOOT1/NOOT2 showed partial dependency on LSH1/LSH2 in the loss and gain of function context. NF-YA1 has been shown to regulate the expression of STY-1 transcription factors, which in turn promote expression of the YUC auxin biosynthesis genes and consistently STY-1 and YUC genes were downregulated in the lsh mutants. Related to this, it was found that several genes involved in auxin transport and conjugation, but also cell cycle regulators including A-type and B-type cyclins and the endoreduplication regulator CSS52B were also dependent on LSH1/LSH2. Many of these genes were constitutively upregulated by overexpression of LSH1/LSH2 and were identified as putative direct targets of LSH1 in the ChiP-Seq experiment (FIG. 4B). Together the RNA-Seq data suggests that LSH1/2 promotes cell proliferation in the cortical derived cell layers via the upregulation of NF-YA1 and its downstream targets.

Cytokinin signalling is required and sufficient for nodule initiation and development even under non-symbiotic conditions which is in stark contrast to its inhibitory effects on the initiation and early development of lateral roots. More recently, it has been shown that cytokinin signalling is required for endosymbiotic host cell colonization by facilitating the switch from mitotic cell proliferation to endoreduplication via the upregulation of CSS52A. Surprisingly, however, we found that genes with a function in cytokinin signaling and biosynthesis were strongly affected by the loss and gain of LSH1/LSH2 function and identified as putative direct ChiP-Seq targets of LSH1, including CRE1, the B-type RESPONSE REGULATORS RR9 and RR11 and members of the LONELY GUY (LOG) gene family (FIG. 4B). Promotion of cytokinin signalling provides further evidence that LSH1/LSH2 function in the establishment of an organ identity that differentiates symbiotic root nodules from lateral roots.

Previously, members of the LSH transcription factor family, closely related to MtLSH1 and MtLSH2, have been characterized to function together with the transcriptional co-activators BLADE-ON-PETIOLE in the shoot where they control the complexity of inflorescences and leaves and the internal asymmetry of floral organs. Consistently, we found a subset of developmental regulators that were upregulated in response to rhizobial spot inoculation in an LSH-dependent manner and also found to be expressed in shoot tissues of M. truncatula (data extracted from the MtExpress Medicago Gene expression atlas; (Carrere et al., 2021)). Several of these LSH-dependent and shoot-expressed genes have been previously annotated to function as regulators of organ growth and organ boundaries such as KLUH and PETAL LOSS (Anastasiou et al., 2007; Brewer et al., 2004). We also found a set of rhizobial-repressed genes that were LSH1/LSH2-dependent, including two members of the PLETHORA (PLT) root meristem regulator family, PLT1 and PLT2 (Franssen et al., 2015). Together, the present combined RNA-Seq and ChiP-Seq analyses demonstrate the LSH genes as major regulators of nodulation that are necessary and sufficient for the up-regulation of cytokinin signalling and nodule-specific regulators such as NF-YA1 and the recruitment of regulators with pleiotropic functions in shoot and symbiotic nodule development, including NOOT1/NOOT2.

Together, the RNA-Seq analysis of gain and loss of function LSH1/LSH2 lines identified LSH genes as key regulators of rhizobial symbiosis that are required and sufficient for both, the up-regulation of nodule-specific organ identity regulators such as NF-YA1 and the recruitment of shoot-related regulators with a function in nodule organogenesis including NOOT1/2.

### Example 5: LSH1/LSH2 Partly Functions Through the Cortical Activation of NF-YA1

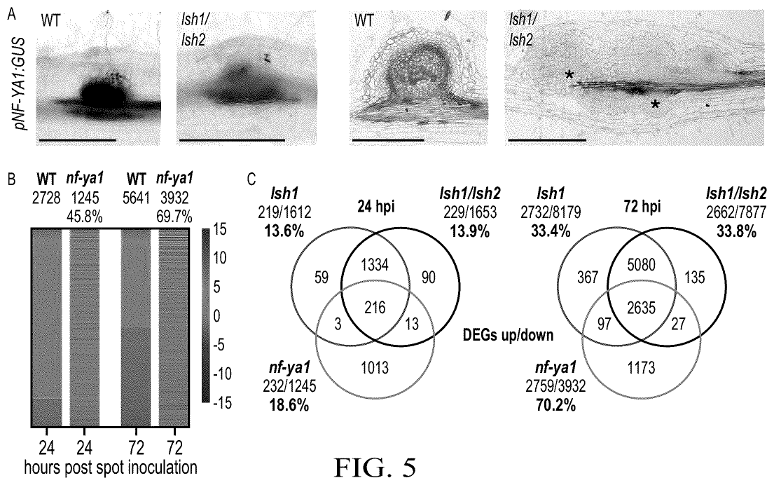

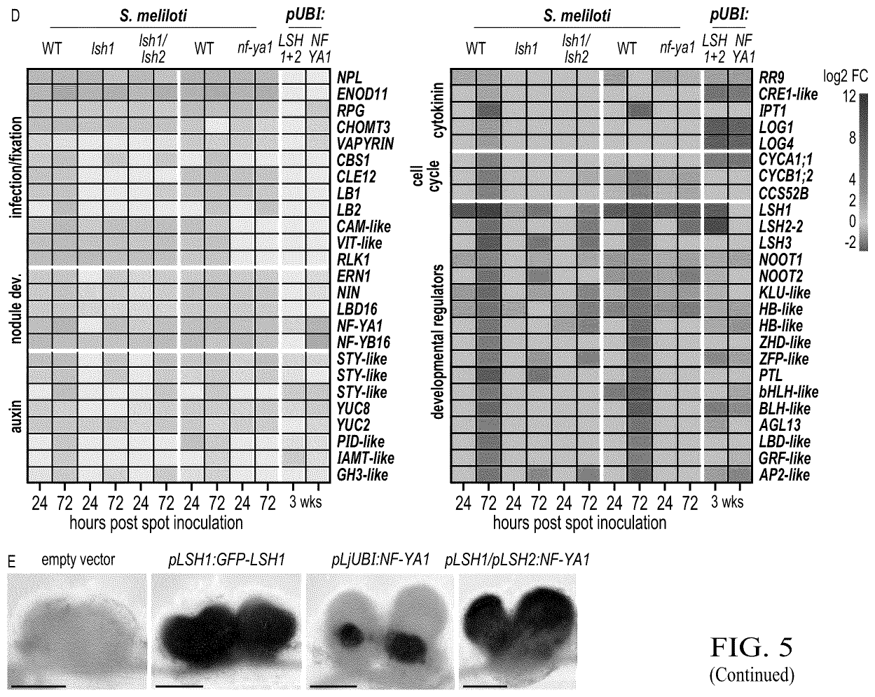

To understand the spatial context of LSH1/LSH2 function, promoter GUS analysis was performed in hairy roots expressing pNF-YA1:GUS-tNF-YA1 in wild-type and lsh1/lsh2 background. pNF-YA1:GUS-tNF-YA1 showed expression in wild type in the inner tissue layers at the base of the developing nodule and in the nodule primordium (FIG. 5A). In the lsh1/lsh2 mutant loss of NF-YA1 expression in the nodule primordium, but the maintenance of its expression in the tissue layers at the base of the nodule was observed (FIG. 5A). Such tissue-specific control of NF-YA1 is consistent with the partial LSH1/LSH2 dependency for NF-YA1 induction observed in the RNA-Seq (FIG. 4B).

### Example 6: NF-YA1 in Part Rescues Nitrogen Fixation in the Lsh1 Lsh2 Nodule Phenotype

Previously, NF-YA1 has been characterized to play a crucial role in promoting cell proliferation, host cell differentiation and endosymbiotic colonization in the primordium cell layers that are derived from the mid-cortex of the primary root. Consistent with this, very similar phenotypes were observed between lsh1/lsh2 and nf-ya1, with an increased ratio of white to blue pNifH-GUS expressing nodules, an increase in nodule number (FIGS. 2A-B and 8), and an increased ratio of aborted cortical infection threads (FIGS. 3A-B). Furthermore, a reduction of cell divisions and cell layers in the nf-ya1 nodule primordia was observed similar to lsh1/lsh2 (FIG. 3C). However, there are also differences between lsh1/lsh2 and nf-ya1 nodules, especially in the overall nodule morphology and maintenance of pNifH-GUS expression.

To further investigate the commonalities and differences in LSH1/LSH2 and NF-YA1 functions, RNA-Seq was performed on rhizobial spot inoculated nf-ya1 and WT root sections at 24 and 72 hpi and compared the gene dependencies of rhizobial-induced genes between NF-YA1 and LSH1/LSH2 (FIGS. 5B, 5C, FIG. 8). NF-YA1 controls a comparatively smaller subset of the rhizobial-induced genes than LSH1/LSH2: 46% and 70% of rhizobial-responsive genes were dependent on NF-YA1 at 24 hpi and 72 hpi, respectively (FIG. 5B, FIG. 8): compared to >90% for LSH1/LSH2 (FIG. 4A). While there was a surprisingly small overlap of <20% between all NF-YA1-dependent and LSH1/LSH2-dependent genes at 24 hpi, the overlap of NF-YA1-dependent genes increased to 71% at 72 hpi, (FIG. 5C). In addition, NF-YA1 was constitutively expressed in hairy roots under non-symbiotic conditions (LjUBI:NF-YA1). Genes that showed strong transcriptional responses in the gain and loss of LSH and NF-YA1 further highlight the role of local auxin biosynthesis, transport and conjugation, cytokinin signaling and cell cycle regulation during early nodulation, but also include several shoot-expressed growth regulators (FIG. 5D). Furthermore, this dataset confirmed that the expression of LSH1/LSH2 is not dependent on NF-YA1 (FIGS. 1B and 5D). Together this suggests that these regulators have independent, additive functions at the early nodule primordium stage and converge on similar regulatory pathways at the timepoint of early cortical infection and nodule differentiation. Based on this, it was hypothesized that these regulators act within similar pathways and that the reduced cortical expression of NF-YA1 might at least in part explain the reduced bacterial colonization and N-fixation phenotype observed in lsh1/lsh2. To test this NF-YA1 was ectopically expressed under the constitutive LjUBI promoter or under the pLSH1 and pLSH2 promoters in lsh1/lsh2 roots. Both modes of NF-YA1 expression resulted in a partial rescue of lsh1/lsh2, leading to 25% of nodules with functional N-fixation, based on pNifH-GUS (FIG. 5E), revealing a functional link between LSH1/LSH2 and NF-YA1 during nodule organogenesis. Together, this provides evidence that LSH1 and LSH2 are required to specifically promote the cortical expression of NF-YA1 during nodule organogenesis.

### Example 7: LSH1/2 and NOOT1/2 Function in the Same Pathway During Nodule Organogenesis

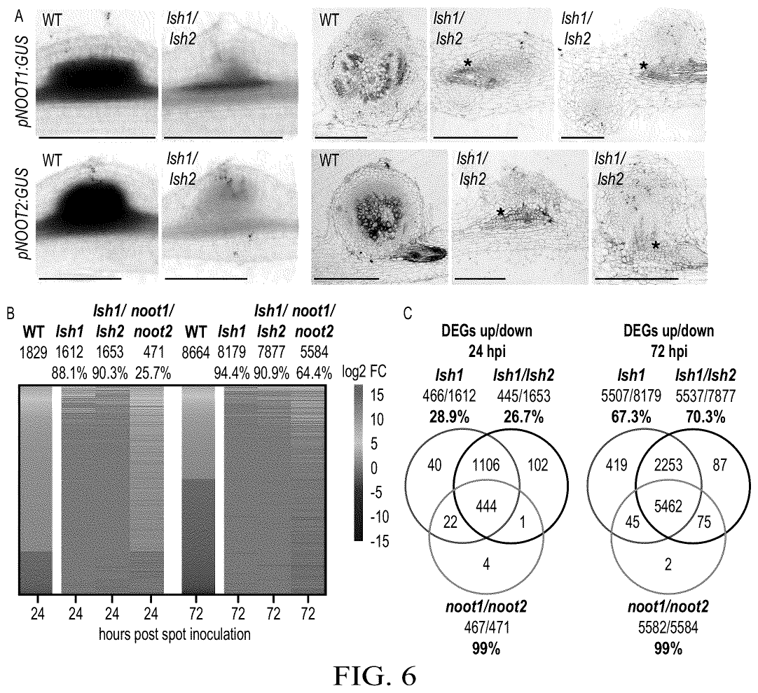

RNA-Seq results also suggested a dependency of NOOT1/NOOT2 expression on LSH1/LSH2. To validate this, promoter GUS analysis was performed in hairy roots expressing pNOOT1:GUS-tNOOT1 and pNOOT2:GUS-tNOOT2 in wild type and lsh1/lsh2. Both NOOT reporters showed expression in the inner tissue layers at the base of the developing nodule and in the nodule primordium in the wild type (FIG. 6A), but in lsh1/lsh2, a moderate reduction in expression of NOOT1 and a loss of expression of NOOT2 in nodule primordia was observed (FIG. 6A).

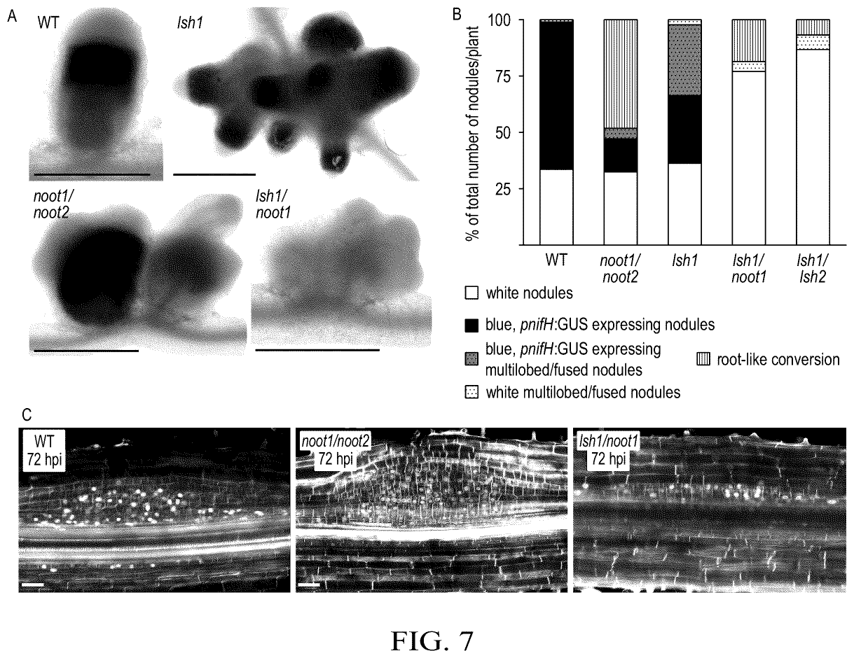

It has previously been shown that several orthologs of the BOP (NOOT) genes function together with members of the LSH family to regulate organ development in the shoot. To test whether LSH and NOOT function together within the same regulatory pathway during symbiotic nodule development, noot1/noot2 was included in time-resolved expression and functional analyses. Unlike lsh1/lsh2 nodule primordia which showed a clear reduction in the periclinal cell divisions of the root cortex, noot1/noot2 primordia showed cell cycle activities comparable or greater than wild type (FIGS. 3C and 7B) and wild-type rhizobial infection. However, at later stages of nodule development, noot1/noot2 showed similar defects to lsh1/lsh2 in rhizobial colonization, resulting in a large proportion of partially or completely uncolonized nodules (FIGS. 3A, 3B, 9A, 9B).

Loss of NOOT1/NOOT2 affects a much smaller subset of the rhizobial-induced gene set than the loss of LSH1/LSH2: 25.75% and 64.45% of rhizobial-responsive genes were not differentially expressed in the noot1/noot2 mutant at 24 hpi and 72 hpi, respectively (FIG. 6B), compared to >90% in lsh1/lsh2 (FIG. 4A). There was a 99% overlap between the genes that were not responding to rhizobial inoculation in the lsh1/lsh2 and in the noot1/noot2 mutants, suggesting that the effect on gene expression caused by loss of NOOT1/NOOT2 is completely embedded in the LSH1/LSH2 function (FIG. 6C). The constitutive expression of NOOT1/NOOT2 (pLjUBI:GFP-NOOT1 pLjUBI:GFP-NOOT2) revealed a substantial (75%) overlap with genes induced by overexpression of LSH1/LSH2 (FIG. 8B, C). Genes that showed strong transcriptional responses in the gain and loss of LSH and NOOT further highlight the role of growth regulators with pleiotropic functions in shoot development, auxin and cytokinin signaling and the requirement for the repression of root meristem regulators such as PLT1 and PLT2 during nodulation (FIG. 8A). LSH1/LSH2 control the expression of NOOT1/NOOT2 genes during nodulation, but NOOT1/NOOT2 has no effect on LSH1 or LSH2 expression (FIG. 4B and FIG. 8A). These studies suggest that NOOT1/NOOT2 function downstream of LSH1/LSH2 and the lack of their expression, at least in part explains the lsh1/lsh2 phenotype, especially in the later stages of nodule development. Consistent with this genetic interactions between LSH and NOOT were observed, with a lsh1/noot1 double mutant recapitulating the phenotype of a lsh1/lsh2 double mutant (FIGS. 7A, 7B, 9A-C). A striking aspect of the noot mutants are the emergence of lateral roots from the tip of nodules. This phenotype was observed in lsh1/lsh2, but at a lower frequency to that observed in noot1/noot2 (FIGS. 7A, 7B, 9C), consistent with the loss of repression of root meristem genes such as PLT1/PLT2 in both double mutants (FIG. 4B and FIG. 8A). The phenotypic resemblance between the lsh1/noot1 and lsh1/lsh2 mutants was also observed at earlier timepoints, where we observed early infection defects and a clear reduction in the periclinal cell divisions of cells derived from the root cortex in lsh1/noot1 as initially observed in the lsh1/lsh2 mutant (FIGS. 3C, 7C, 9A, 9B). This indicates that LSH1 and LSH2 control NOOT1 and NOOT2 and this regulation partially explains the loss of function lsh1/lsh2 phenotype.

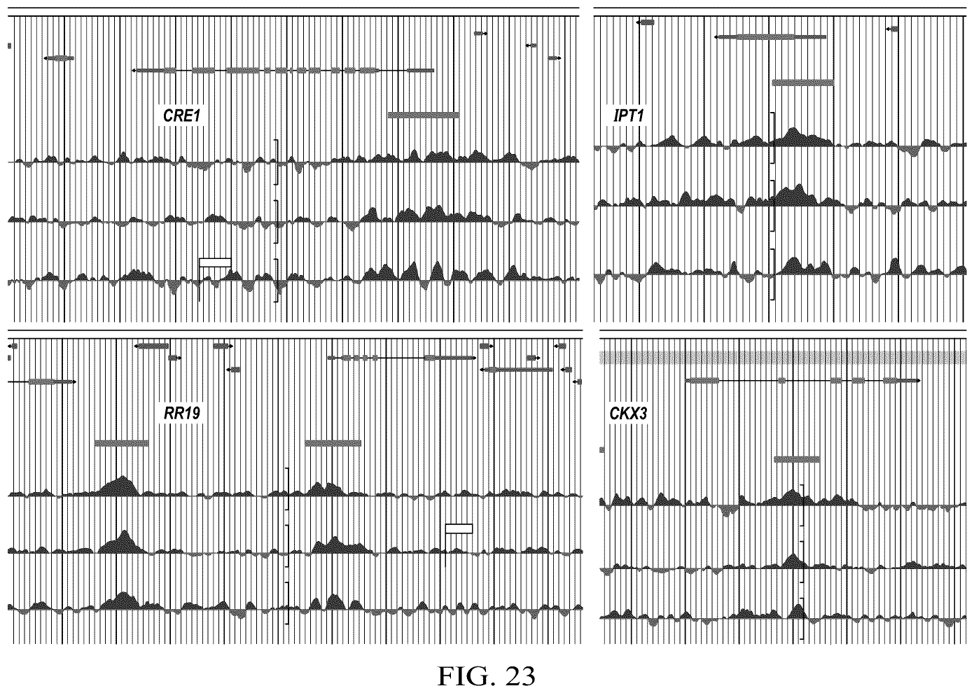

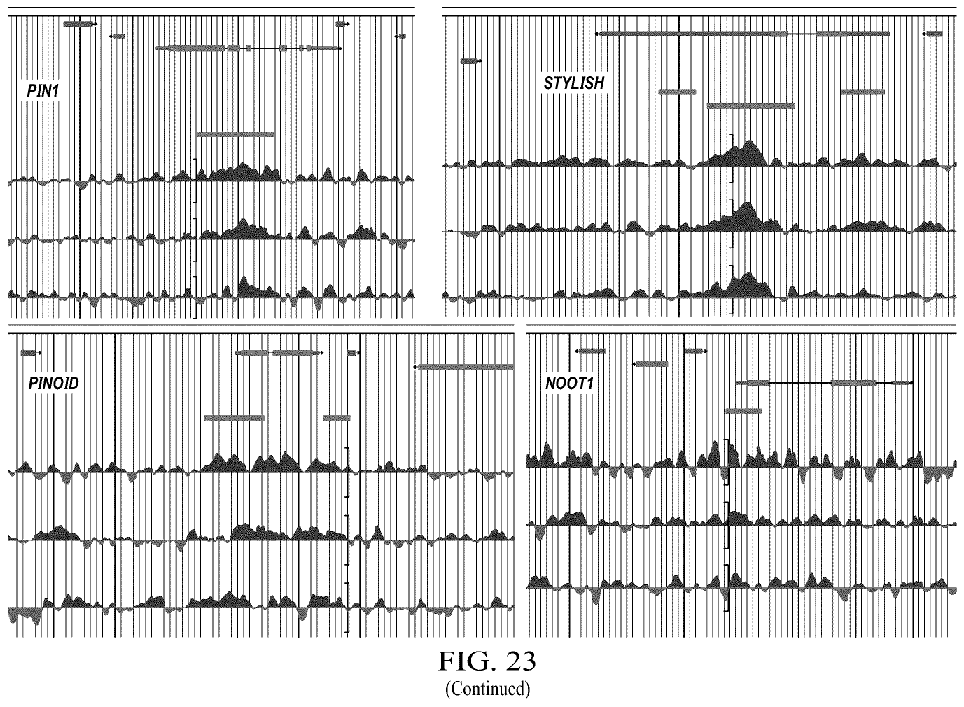

Putative direct targets of LSH1 were further investigated using ChiP-Seq (FIG. 23). Peaks shown in FIG. 23 are high confidence targets that were found in at least 3 out of 4 biological replicates, and include CRE1, IPT1, RR19, CKX3, PIN1, STYLISH, PINOID, and NOOT1. These results indicate that the LSH1 transcription factor binds directly to the DNA regions of cytokinin and auxin signalling and biosynthesis genes, and the nodule identity regulator NOOT1.

### Example 8: Overexpression of LSH1 in Plants

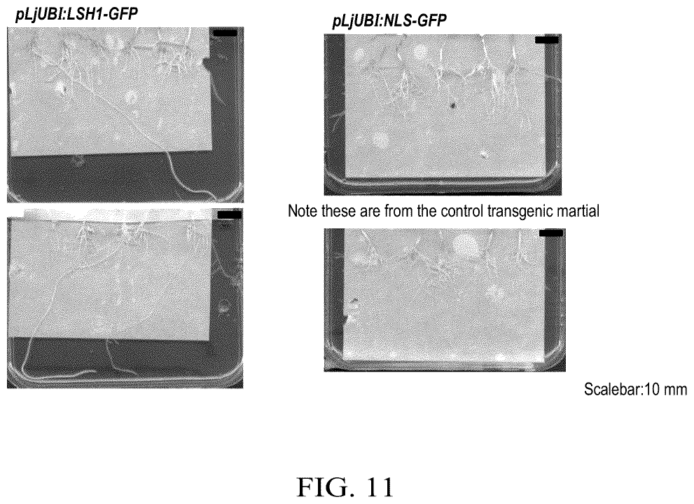

A Medicago truncatula plant cell was transformed with a vector comprising a sequence encoding LSH1 (SEQ ID NO: 1) under control of a heterologous plant promoter (pLjUBI:GFP-LSH1). Transformed plant cells were regenerated to produce LSH1 over-expressing plants. Ectopic expression of LSH1 resulted in altered transcriptional profile of nodulation genes. Additionally, altered root structures were observed as compared to control plants, including increased root length and diameter (FIG. 11). Overexpression of LSH1 also modified lateral root primordia development (FIG. 12) as compared to control plants. The LSH1-overexpressing plants were further inoculated with bacteria to evaluate rhizobial infection and nodule formation. Inoculation resulted in altered rhizobial infection patterns and nodulation structures including cluster-like multi-lobed nodules (FIG. 13).

### Example 9: Overexpression of LSH2 in Plants

A plant cell is transformed with a vector comprising a sequence encoding LSH2 (SEQ ID NO: 3) under control of a heterologous plant promoter. Transformed plant cells are regenerated to produce LSH2 over-expressing plants, showing altered transcriptional profile of nodulation genes; altered root structures (e.g., increased root length and diameter); and modified lateral root primordia development, similar to the results described in Example 8. LSH2 overexpressing plants will also be inoculated with bacteria to evaluate rhizobial infection and nodule formation, showing altered rhizobial infection patterns and nodulation sutures including cluster-like multi-lobed nodules, similar to the results described in Example 8.

### Example 10 Simultaneous Overexpression of LSH1 and LSH2 in Plants

A Medicago truncatula plant cell was transformed with a vector comprising a sequence encoding LSH1 and LSH2 (SEQ ID NO: 1 and SEQ ID NO: 3, respectively) under control of a heterologous plant promoter(s). Transformed plant cells were regenerated to produce plants over-expressing LSH1 and LSH2. LSH1 and LSH2 overexpressing plants were inoculated with bacteria to evaluate rhizobial infection and nodule formation, showing altered rhizobial infection patterns and nodulation structures (FIG. 14).

### Example 11: Plants with Modified LSH1 Activity

A non-legume plant cell is genomically modified to introduce a modification to an endogenous sequence encoding LSH1 (SEQ ID NO: 1). Modified plant cells are regenerated to produce plants with altered LSH1 activity compared with a control plant not comprising the modification. Plants with altered LSH1 activity show altered transcriptional profile and altered root structures such as increase in root length and diameter, and exhibit development of modified lateral roots similar to nodules, in addition to enhanced interactions with rhizobia.

### Example 12: Plants with Modified LSH2 Activity

A non-legume plant cell is genomically modified to introduce a modification to an endogenous sequence encoding LSH2 (SEQ ID NO: 3). Modified plant cells are regenerated to produce plants with altered LSH2 activity compared with a control plant not comprising the modification. Plants with altered LSH2 activity show altered transcriptional profile and altered root structures such as increase in root length and diameter, and exhibit development of modified lateral roots similar to nodules in addition to enhanced interactions with rhizobia.

### Example 13: Assessment of Gene Function in Stably Transformed Roots

For preliminary functional assessments of gene functions in the root, a stable root transformation system for barley (STARTS) (Imani et al., 2011) was used. This method is based on the callus produced from the scutellum of the immature embryo. By using Agrobacterium tumefaciens-mediated transformation and then transferring the calli directly to the barley root induction medium, which contains liquid endosperm of coconut fruits, sucrose, and 2 mg/l Indole-3-butyric acid (IBA), calli can be regenerated from transformed roots in 6 weeks for further rapid preliminary gene functional analysis.

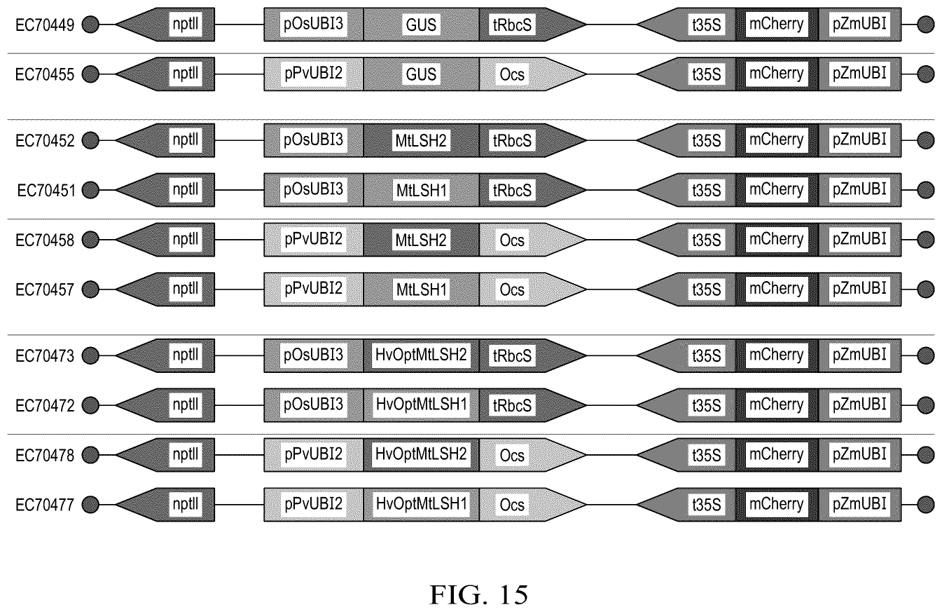

6-week-old STARTS plates were wrapped with foil and kept at 25 degrees Celsius in a growth chamber for 2 weeks for the transformed roots to grow further. The first batch of harvesting was from 8-week-old STARTS transformed roots. STARTS transformed roots were observed under a Leica MZ10F dissecting microscope with a fluorescent light source and mCherry fluorescent light filter. Roots with mCherry fluorescent signals were labelled, and NLSs were circled on the plates. All visible NLSs from negative GUS control, pOsUbi::MtLSH1, pOsUbi::MtLSH2, and pOsUbi::HvOptMtLSH1 (FIG. 15) transformed roots were harvested and immediately mounted with 50% glycerol for confocal imaging. After imaging, these NLS were immediately fixed with 4% PFA in PBS solution for 1 hour and then transferred to ClearSee solution for tissue clearing.

Immediately after the first harvest, the calli with regenerated roots and shoots were transferred to enriched medium plates (MS medium or MODFP medium) for regenerated STARTS plants to grow further for seven days. For the Medicago LSH1 and LSH2 genes (MtLSH1, MtLSH2), STARTS regenerated plants were transferred to a buffered nodulation medium (BNM)-N+P (nitrogen deficiency condition). After growing in BNM-N+P for four days, 50 uM 2,4-Dichlorophenoxyacetic acid (2,4-D) solution was sprayed on the regenerated plants for auxin treatment. For the barley codon-optimized version of LSH1 and LSH2 genes (HvOptMtLSH1 and HvOptMtLSH2), STARTS regenerated plants were directly transferred to BNM-N+P medium with 50 uM 2,4-D.

### Example 14: MtLSH1 or HvOptMtLSH1 Alters Barley Lateral Organ Development

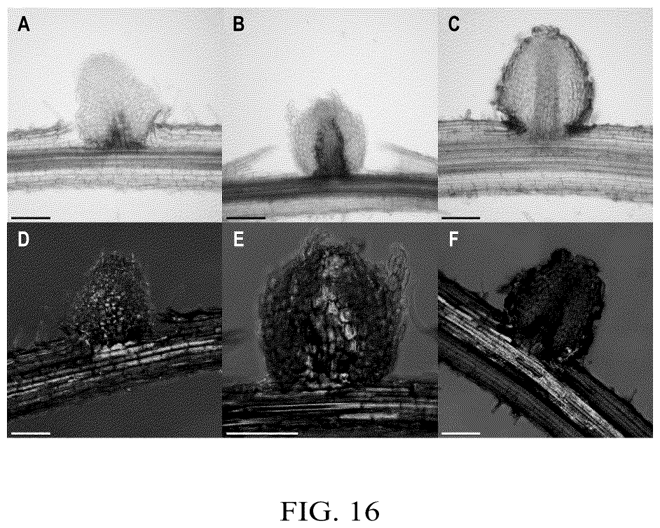

To evaluate the effect of introducing the Medicago LSH1 and LSH2 genes (MtLSH1, MtLSH2) and the barley codon-optimized version of LSH1 and LSH2 genes (HvOptMtLSH1 [SEQ ID NO:7] and HvOptMtLSH2 [SEQ ID NO:8]) on barley root organogenesis, constitutive overexpressing constructs of these genes driven by rice ubiquitin promoters were created (FIG. 15). No obvious differences were initially observed between using pOsUBI3 and pPvUBI2. Therefore results are presented as pUbi with the data combined. The pZmUBI::mCherry (FIG. 15) serves as a visual maker for the construct to help identify the STARTS roots that were successfully transformed. The roots with mCherry signals are marked and collected for further analysis (FIG. 16).

Overexpressing MtLSH1 or MtLSH2 in barley roots alters the organogenesis of barley lateral roots (FIG. 16, 17). The NLS harvested from negative GUS control have a central vasculature, well-defined apical meristem and an apparent root tip (FIG. 16A, 16D). The NLS harvested from both pOsUbi::MtLSH1 and pOsUbi::MtLSH2 transformed roots have altered overall morphology and more spherical shape, but still have a central vasculature and a persistent meristem (FIG. 16B-C, 16E-F).

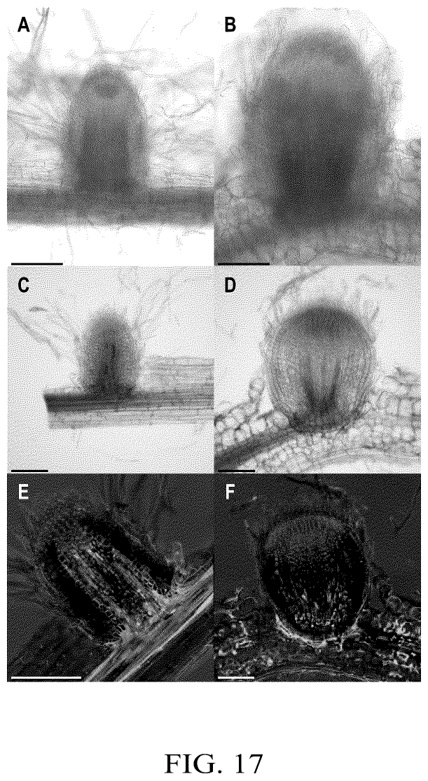

Intriguingly, the effect of introducing MtLSH1 in barley roots appeared to be more pronounced on the NLSs when the transformed barley roots were grown in nitrogen deficiency conditions (BNM-N+P medium) and with the supplement of 50 μM 2,4-D. Based on the harvest 2 results after auxin treatments, the NLSs harvested from negative GUS control looks like a stunted lateral root, which have a broad base, central vasculature, and an apparent root tip (FIG. 17A, 17C, 17E). On the other hand, the NLSs harvested from pUbi::MtLSH1 transformed roots have an enlarged spherical shape, broad base, and expanded meristem (FIG. 17B, 17D, 17F). Surprisingly, the NLSs had multiple vascular bundles branching out from the base and connecting to the primary root vasculature, similar to the morphology of Medicago nodules (FIG. 17D).

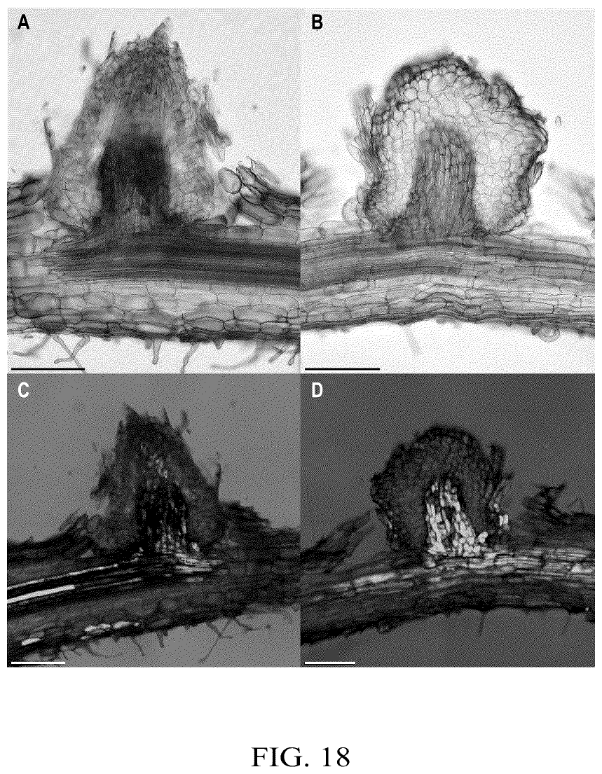

To evaluate whether codon optimization would promote the expression and function of MtLSH genes in barley, the barley codon-optimized version of MtLSH1 and MtLSH2 (HvOptMtLSH1 and HvOptMtLSH2) were introduced. Overexpressing HvOptMtLSH1 in barley roots also alters the organogenesis of barley lateral roots, but leads to different morphological changes (FIG. 18, 19). Based on the harvest 1 results, the NLS harvested from negative GUS control have a central vasculature, well-defined apical meristem and an apparent root tip (FIG. 18A, 18C). The NLS gathered from pOsUbi::HvOptMtLSH1 transformed roots have altered overall morphology, lack a persistent meristem, and have a more spherical shape but still have a central vasculature (FIG. 17B, 17D).

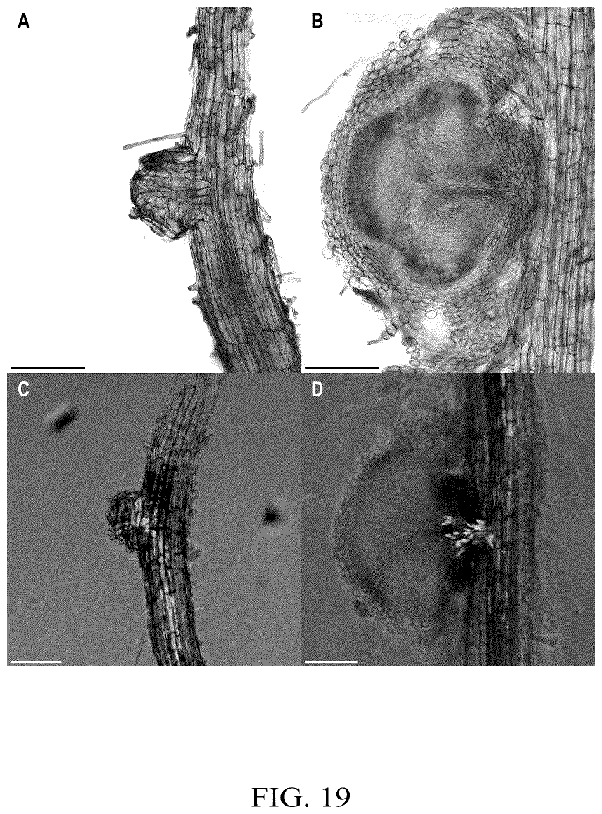

Interestingly, the effect of introducing HvOptMtLSH1 in barley roots also appeared to be more pronounced on the NLSs when the transformed barley roots were grown in BNM-N+P medium and with the supplement of 50 μM 2,4-D. Based on results after auxin treatments, the NLSs harvested from negative GUS control have a small spherical shape and central vasculatures (FIG. 19A, 19C). On the other hand, the NLSs harvested from Ubi::HvOptMtLSH1 transformed roots have a multi-lobed structure, dispersed meristem, and diffused vascular bundles (FIG. 19B, 19D).

### Example 15: No Significant Change in NLS Numbers and Frequency is Observed in MtLSH1 or HvOptMtLSH1 Transformed Roots

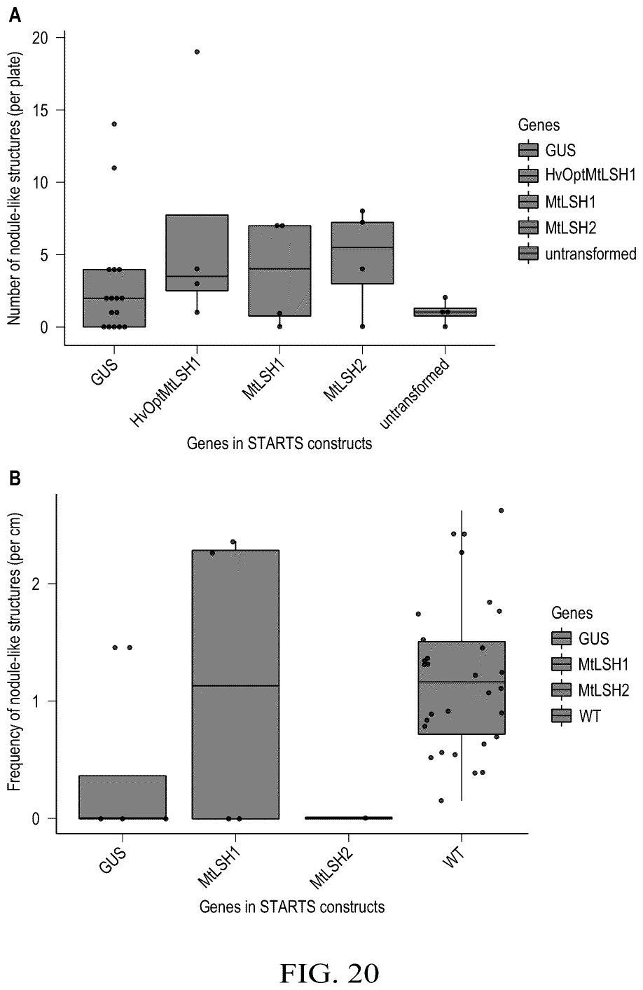

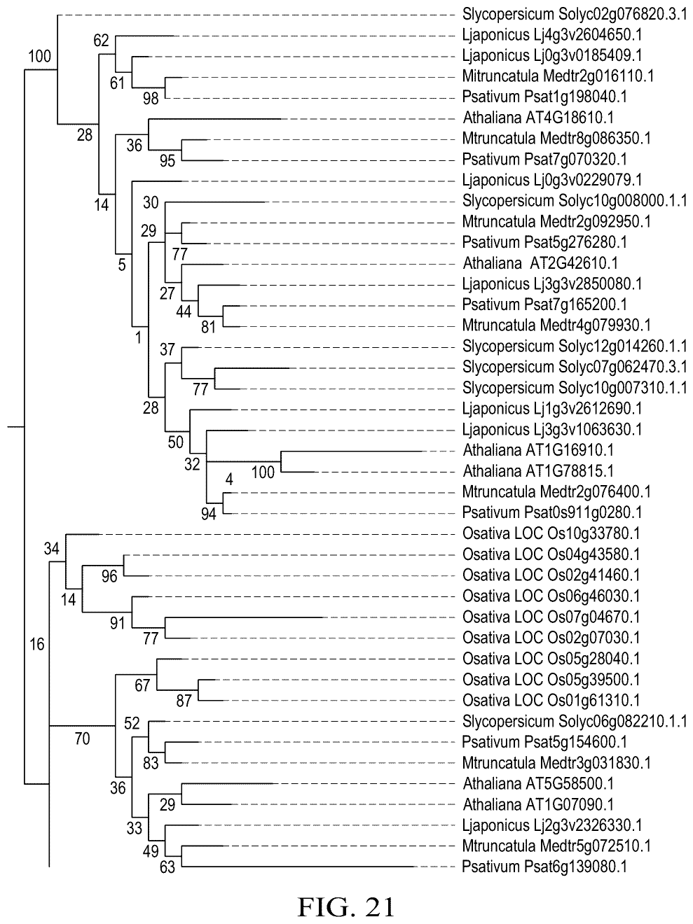

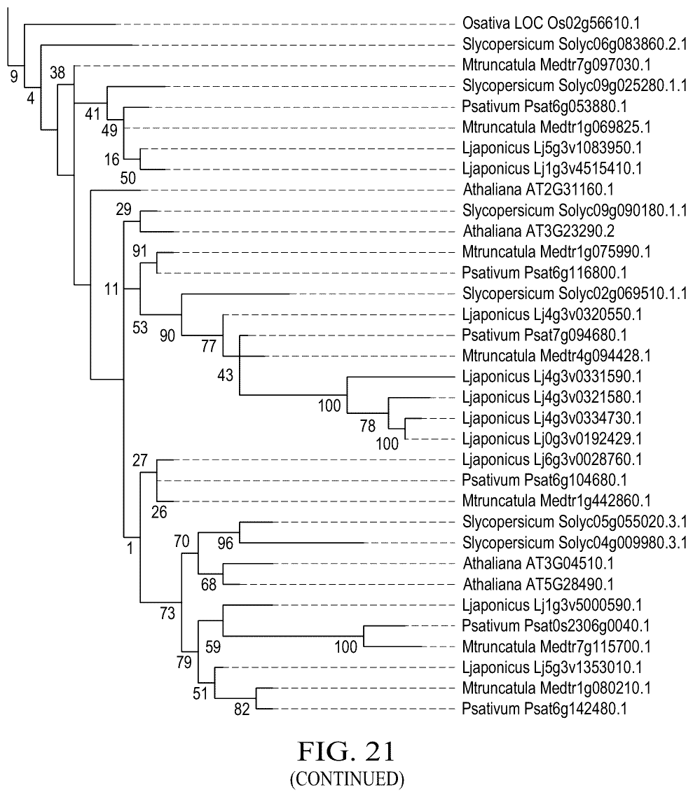

To evaluate whether introducing MtLSH1 or HvOptMtLSH1 would influence the frequency of barley lateral organ initiation, the NLS numbers and frequency were also quantified. No significant change in NLS numbers and frequency were observed in MtLSH1 or HvOptMtLSH1 transformed roots before or after auxin treatments (FIG. 20).

### Example 16: Cassava Plants with Modified LSH1 Activity

A cassava plant cell is genomically modified to introduce a modification to an endogenous sequence encoding LSH1 (SEQ ID NO: 1). Modified plant cells are regenerated to produce plants with altered LSH1 activity compared with a control plant not comprising the modification. Plants with altered LSH1 activity show altered transcriptional profile and altered root structures such as increase in root length and diameter, and exhibit development of modified lateral roots similar to nodules, in addition to enhanced interactions with rhizobia.

### Example 17: Cassava Plants with Modified LSH2 Activity

A cassava plant cell is genomically modified to introduce a modification to an endogenous sequence encoding LSH2 (SEQ ID NO: 3). Modified plant cells are regenerated to produce plants with altered LSH2 activity compared with a control plant not comprising the modification. Plants with altered LSH2 activity show altered transcriptional profile and altered root structures such as increase in root length and diameter, and exhibit development of modified lateral roots similar to nodules in addition to enhanced interactions with rhizobia.

### Example 18: A Putative NIN-Binding Site in the LSH1 Exon

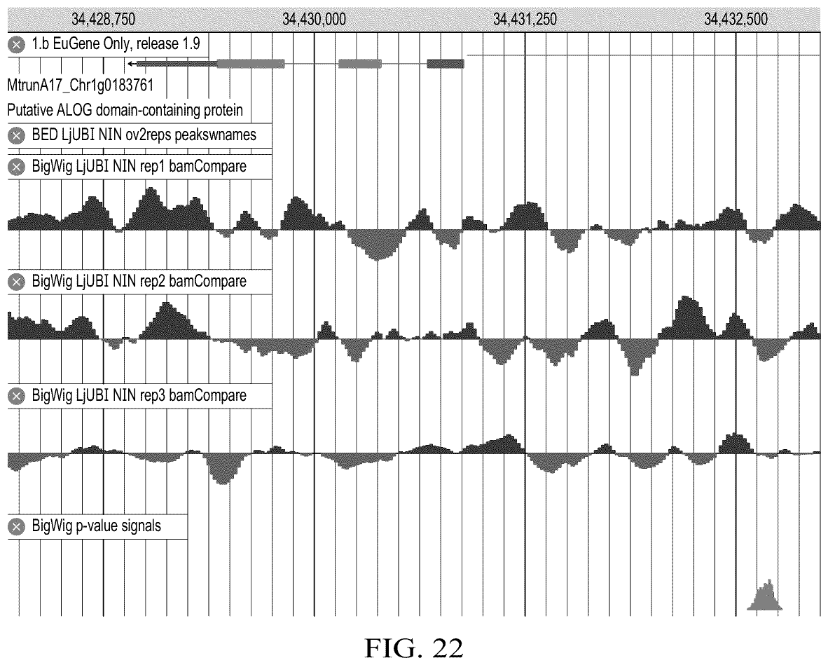

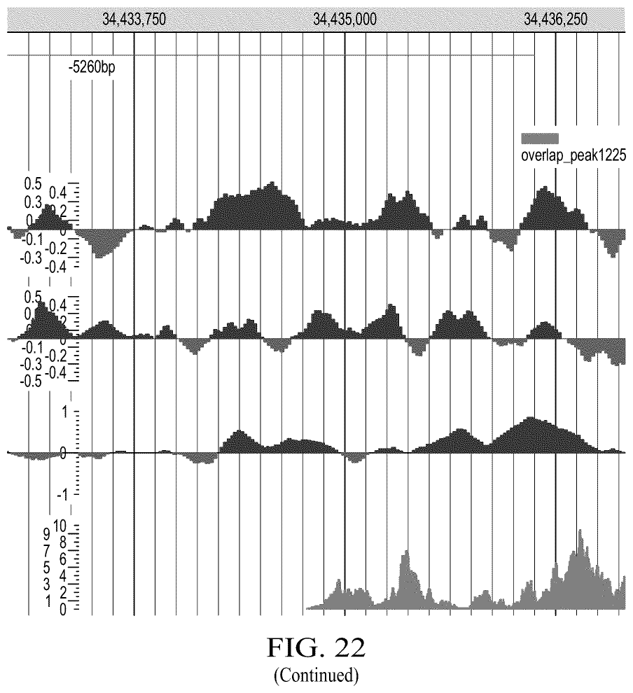

Chip-Seq using hairy roots expressing LjUBI:GFP-NIN under nonsymbiotic conditions revealed a high confidence DNA binding site (found in >50%, 2 out of 3 biological replicates) 5260 bp upstream of LSH1 (FIG. 22). Panels from the top indicate Eugene annotation of Medicago genome version 5, confident peak called region, the reads from LjUBI:GFP-NIN ChIP replicates mapped to the genome relative to the controls, and the pooled p-value significance signal.

Having illustrated and described the principles of the present invention, it should be apparent to persons skilled in the art that the invention can be modified in arrangement and detail without departing from such principles. We claim all modifications that are within the spirit and scope of the claims. All publications and published patent documents cited herein are hereby incorporated by reference to the same extent as if each individual publication or patent application is specifically and individually indicated to be incorporated by reference.

1. A recombinant DNA molecule comprising a heterologous promoter operably linked to a polynucleotide segment encoding a light sensitive short hypocotyl protein or fragment thereof, wherein:
a. said protein comprises the amino acid sequence of SEQ ID NO: 2 or 4 or a fragment thereof;
b. said protein comprises an amino acid sequence having at least about 85%, at least about 90%, at least about 95%, at least about 98%, at least about 99%, or about 100% amino acid sequence identity to SEQ ID NO: 2 or 4 or a fragment thereof;
c. said polynucleotide segment hybridizes under stringent hybridization conditions to a polynucleotide having the nucleotide sequence of SEQ ID NO: 1, 3, 5, 6, 7, or 8 or a fragment thereof; or
d. said polynucleotide segment hybridizes under stringent hybridization conditions to a polynucleotide having the nucleotide sequence having at least about 85%, at least about 90%, at least about 95%, at least about 98%, at least about 99%, or about 100% nucleotide sequence identity to SEQ ID NO: 1, 3, 5, 6, 7, or 8 or a fragment thereof.

2. The recombinant DNA molecule of claim 1, wherein:
a. said recombinant DNA molecule is expressed in a plant cell to produce an increase in intercellular cortical infection, an increase in intracellular colonization by nitrogen-fixing bacteria, or an increase in nitrogen-fixation by bacteria; or
b. said recombinant DNA molecule is in operable linkage with a vector, and said vector is selected from the group consisting of a plasmid, phagemid, bacmid, cosmid, and a bacterial or yeast artificial chromosome.

3. The recombinant DNA molecule of claim 1, present within a host cell, wherein said host cell is selected from the group consisting of a bacterial cell and a plant cell.

4. The recombinant DNA molecule of claim 3, wherein said bacterial host cell is from a genus of bacteria selected from the group consisting of: Agrobacterium, Rhizobium, Bacillus, Brevibacillus, Escherichia, Pseudomonas, Klebsiella, Pantoea, and Erwinia.

5. The recombinant DNA molecule of claim 4, wherein said Bacillus is Bacillus cereus or Bacillus thuringiensis, said Brevibacillus is a Brevibacillus laterosperous, and said Escherichia is a Escherichia coli.

6. The recombinant DNA of claim 2, wherein said plant cell is a dicotyledonous or a monocotyledonous plant cell.

7. The recombinant DNA of claim 6, wherein said plant cell is selected from the group consisting of an alfalfa, almond, Bambara groundnut, banana, barley, bean, black currant, broccoli, cabbage, blackberry, brassica, canola, carrot, cassava, castor, cauliflower, celery, chickpea, Chinese cabbage, citrus, coconut, coffee, corn, clover, cotton, cowpea, a cucurbit, cucumber, Douglas fir, eggplant, eucalyptus, flax, forage legume, garlic, grape, hemp, hops, indigo, leek, legume, legume trees, lentil, lettuce, Loblolly pine, lotus, lupin, millets, melons, Medicago spp., nut, oat, olive, onion, ornamental, palm, pasture grass, pea, peanut, pepper, pigeon pea, pine, potato, poplar, pumpkin, pulses, Radiata pine, radish, rapeseed, raspberry, red currant, rice, rootstocks, rye, safflower, shrub, sorghum, Southern pine, soybean, spinach, squash, strawberry, sugar beet, sugarcane, sunflower, sweet gum, sweet potato, switchgrass, tea, tobacco, tomato, triticale, turf grass, walnut, watermelon, wheat, and yam plant cell.

8. A plant or part thereof comprising the recombinant DNA molecule of claim 1.

9. The plant or part thereof of claim 8, wherein said plant is a monocot plant or a dicot plant.

10. The plant or part thereof of claim 9, wherein said plant is selected from the group consisting of an alfalfa, almond, Bambara groundnut, banana, barley, bean, black currant, broccoli, cabbage, blackberry, brassica, canola, carrot, cassava, castor, cauliflower, celery, chickpea, Chinese cabbage, citrus, coconut, coffee, corn, cowpea, clover, cotton, a cucurbit, cucumber, Douglas fir, eggplant, eucalyptus, flax, forage legumes, garlic, grape, hemp, hops, indigo, leek, legume, legume trees, lentil, lettuce, Loblolly pine, lotus, lupin, millets, melons, Medicago spp., nut, oat, olive, onion, ornamental, palm, pasture grass, pea, peach, peanut, pepper, pigeon pea, pine, potato, poplar, pumpkin, pulses, Radiata pine, radish, rapeseed, raspberry, red currant, rice, rootstocks, rye, safflower, shrub, sorghum, Southern pine, soybean, spinach, squash, strawberry, sugar beet, sugarcane, sunflower, sweet gum, sweet potato, switchgrass, tea, tobacco, tomato, triticale, turf grass, walnut, watermelon, wheat, and yam.

11. The plant or part thereof of claim 8, wherein expression of said polynucleotide segment encoding a light sensitive short hypocotyl protein varies over a 24-hour period.

12. The plant or part thereof of claim 11, wherein expression of said polynucleotide segment encoding a light sensitive short hypocotyl protein is increased during the first 12 hours of a 12 hour/12 hour light/dark cycle.

13. The plant or part thereof of claim 11, wherein expression of said polynucleotide segment encoding a light sensitive short hypocotyl protein is increased during the first 6 hours of a 12 hour/12 hour light/dark cycle.

14. A transgenic seed comprising the recombinant DNA molecule of claim 1.

15. A method of producing progeny seed comprising the recombinant DNA molecule of claim 1, the method comprising:
a. planting a first seed comprising the recombinant DNA molecule of claim 1;
b. growing a plant from the seed of step a; and
c. harvesting the progeny seed from the plants, wherein said harvested seed comprises said recombinant DNA molecule.

16. A plant susceptible to intercellular cortical infection or intracellular colonization by nitrogen-fixing bacteria, wherein the cells of said plant comprise the recombinant DNA molecule of claim 1.

17. A method for increasing intercellular cortical infection or intracellular colonization by nitrogen-fixing bacteria in a plant, said method comprising:
a. expressing a light sensitive short hypocotyl protein or fragment thereof having at least about 70%, at least about 80%, at least about 90%, at least about 95%, at least about 99%, or about 100% sequence identity to SEQ ID NO: 2 or 4 in a plant; or
expressing a light sensitive short hypocotyl protein or fragment thereof encoded by a nucleic acid sequence having at least about 70%, at least about 80%, at least about 90%, at least about 95%, at least about 99%, or about 100% sequence identity to SEQ ID NO: 1, 3, 5, 6, 7, or 8 or a fragment thereof; and
b. contacting said plant with an effective amount of one or more rhizobia bacterium, arbuscular mycorrhiza fungi, or a combination thereof.

18. The method of claim 17, wherein:
a. said rhizobia bacterium is selected from the group consisting of: S. meliloti, Mesorhizobium loti, Sinorhizobium meliloti, Sinorhizobium fredii, Rhizobium sp. IRBG74 and NGR234, and Bradyrhizobium sp.; or
b. said arbuscular mycorrhiza fungi is selected from the group consisting of: R. irregularis, Rhizophagus intraradices, Glomus mosseae, and Funneliformis mosseae.

19. A modified plant, plant seed, plant part, or plant cell, comprising a genomic modification that modulates the activity of LSH1 or LSH2, as compared to the activity of LSH1 or LSH2 in an otherwise identical plant, plant seed, plant part, or plant cell that lacks the modification.

20. The modified plant, plant seed, plant part, or plant cell of claim 19, wherein the modification is present in at least one allele of an endogenous LSH1 or LSH2 gene.

21. The modified plant, plant seed, plant part, or plant cell of claim 20, wherein the genomic modification is in an endogenous LSH1 or LSH2 gene encoding a protein having at least 70%, at least 75%, at least 80%, at least 85%, at least 90%, at least 95%, at least 96%, at least 97%, at least 98%, at least 99%, at least 99.5%, or 100% sequence identity to SEQ ID NO: 2 or 4.

22. The modified plant, plant seed, plant part, or plant cell of claim 20, wherein the modification is in a transcribable region of the LSH1 or LSH2 gene.

23. The modified plant, plant seed, plant part, or plant cell of claim 20, wherein the plant, plant seed, plant part, or plant cell is heterozygous for the modification.

24. The modified plant, plant seed, plant part, or plant cell of claim 20, wherein the plant, plant seed, plant part, or plant cell is homozygous for the modification.

25. The modified plant, plant seed, plant part, or plant cell of claim 20, wherein the modification comprises a deletion, an insertion, a substitution, an inversion, a duplication, or a combination of any thereof.

26. The modified plant, plant seed, plant part, or plant cell of claim 20, wherein the modification comprises a deletion of at least 1, at least 3, at least 5, at least 10, at least 15, at least at least 25, at least 30, at least 35, at least 40, at least 45, at least 50, at least 55, at least 60, at least 65, at least 70, at least 75, at least 80, at least 85, at least 90, at least 95, at least 100, at least 125, or at least 150 consecutive nucleotides.

27. The modified plant, plant seed, plant part, or plant cell of claim 19, wherein the plant, plant seed, plant part, or plant cell comprises a chromosomal sequence in the LSH1 or LSH2 gene that has at least 70%, at least 75%, at least 80%, at least 85%, at least 90%, at least 95%, at least 96%, at least 97%, at least 98%, at least 99%, at least 99.5%, or 100% sequence identity to SEQ ID NO: 1 or SEQ ID NO: 3 in the regions outside of the deletion, the insertion, the substitution, the inversion, or the duplication.

28. A method for producing a plant comprising a modified LSH1 or LSH2 gene, the method comprising:
a) introducing a modification into at least one target site in an endogenous LSH1 or LSH2 gene of a plant cell that modulates the activity of LSH1 or LSH2;
b) identifying and selecting one or more plant cells of step (a) comprising said modification in said LSH1 or LSH2 gene; and
c) regenerating at least a first plant from said one or more cells selected in step (b) or a descendent thereof comprising said modification.

29. A recombinant DNA molecule comprising a DNA sequence selected from the group consisting of:
a) a sequence with at least 85 percent sequence identity to any of SEQ ID NOs: 84-93;
b) a sequence comprising any of SEQ ID NOs: 84-93;
c) a fragment of any of SEQ ID NOs: 84-93, wherein the fragment has gene-regulatory activity; and
d) a fragment of a sequence with at least 85 percent sequence identity to of any of SEQ ID NOs: 84-93, wherein the fragment has gene-regulatory activity;
wherein said sequence is operably linked to a heterologous transcribable DNA molecule.

30. The recombinant DNA molecule of claim 29, wherein said sequence has at least 90 percent sequence identity to the DNA sequence of any of SEQ ID NOs: 84-93 or fragment thereof.

31. The recombinant DNA molecule of claim 30, wherein said sequence has at least 95 percent sequence identity to the DNA sequence of any of SEQ ID NOs: 84-93 or fragment thereof.

32. The recombinant DNA molecule of claim 31, wherein said sequence comprises the DNA sequence of any of SEQ ID NOs: 84-93 or fragment thereof.

33. The recombinant DNA molecule of claim 29, wherein the heterologous transcribable DNA molecule comprises a gene of agronomic interest.

34. A transgenic plant cell comprising the recombinant DNA molecule of claim 29.

35. The transgenic plant cell of claim 34, wherein said transgenic plant cell is a monocotyledonous plant cell.

36. The transgenic plant cell of claim 34, wherein said transgenic plant cell is a dicotyledonous plant cell.

37. A transgenic plant, or part thereof, comprising the recombinant DNA molecule of claim 29.

38. A progeny plant of the transgenic plant of claim 37, or a part thereof, wherein the progeny plant or part thereof comprises said recombinant DNA molecule.

39. A transgenic seed, wherein the seed comprises the recombinant DNA molecule of claim 29.

40. A method of producing a commodity product comprising obtaining a transgenic plant or part thereof according to claim 37 and producing the commodity product therefrom.

41. The method of claim 40, wherein the commodity product is protein concentrate, protein isolate, grain, starch, seeds, meal, flour, biomass, or seed oil.

42. A method of expressing a transcribable DNA molecule comprising obtaining a transgenic plant according to claim 37 and cultivating said plant, wherein the transcribable DNA is expressed.


SEQUENCE LISTING (94 sequences)

SEQ ID NO: 1 - DNA, 669 bp, synthetic construct
  source 1..669: mol_type = other DNA; note = cDNA sequence encoding the LSH1 protein
atggattcaattcaagattttatggactcatgtaactctgacaacagttgcagcctcaccaacagcaccataactaccaacagcagtaacaacaacatcagcaacgccatagttggtagctcttcaccttctggctctaccaccacaagcagccgctatgaaaaccagaaacgccgtgactggaacacttttggccagtacctcaagaatcaccgtcctcctctctccctctccagatgtagcggtgctcatgtgcttgaatttctccggtacttggaccaatttggcaagacaaaagtgcacacaccaatttgtccattctatggacatccaaaccctccagcaccatgtccatgtccattaagacaagcttggggtagtcttgatgcactgataggtcgtttaagggcagcttttgaggaaaatggagggaagccagaaacaaatccatttggtgctagagctgttagactttaccttcgtgaggttcgtgatcttcaatccaaagcaagaggtattagttatgagaagaagaaaaggaaacgtccaccacaacaacaaccacaacagcttcagcttcagcagcaacaaccaatgcaattgcaattgcctcttcatcttcatcatcaccatcaacatcagcttccacctccaggtgcaactcaataa

SEQ ID NO: 2 - protein, 222 aa, Medicago truncatula
MDSIQDFMDSCNSDNSCSLTNSTITTNSSNNNISNAIVGSSSPSGSTTTSSRYENQKRRDWNTFGQYLKNHRPPLSLSRCSGAHVLEFLRYLDQFGKTKVHTPICPFYGHPNPPAPCPCPLRQAWGSLDALIGRLRAAFEENGGKPETNPFGARAVRLYLREVRDLQSKARGISYEKKKRKRPPQQQPQQLQLQQQQPMQLQLPLHLHHHHQHQLPPPGATQ

SEQ ID NO: 3 - DNA, 645 bp, synthetic construct
  source 1..645: mol_type = other DNA; note = cDNA sequence encoding the LSH2 protein
atggattcaattcaagaatttatagggacatgtaacaatgaaaacttgacctgcaacttcatgaacaacaacaacaacaacactataagtacaaccacaacaagcttaacaacaacaacaacaacagcaagtggaagctcttcttcatctgcagcttcaaccatcatcaacagtcccaacagtagcagccgctatgagaaccagaaacgccgtgactggaacacctttggtcagtacctcaagaatcatcgtccacctctttccctctccaggtgcagcggtgcacatgtccttgaatttctaaggtatttggatcaatttggcaagacaaaagttcacacaccaatttgtccattttatgggcatccaaatccaccagcaccatgtccatgtcctctgaggcaagcatggggaagccttgatgcactcataggtcgtcttcgtgccgcctttgaagaaaatggaggaaaaccagaaacaaatccatttggtgctagagctgtgaggctctaccttcgcgaagttcgcgatcttcaatcgaaagcaagaggtattagttacgagaagaagaaacgaaagcgtccaccaccacaacaacaacaacaagttccaccaccagcacaaggtgcaagtgcaactcattag

SEQ ID NO: 4 - protein, 214 aa, Medicago truncatula
MDSIQEFIGTCNNENLTCNFMNNNNNNTISTTTTSLTTTTTTASGSSSSSAASTIINSPNSSSRYENQKRRDWNTFGQYLKNHRPPLSLSRCSGAHVLEFLRYLDQFGKTKVHTPICPFYGHPNPPAPCPCPLRQAWGSLDALIGRLRAAFEENGGKPETNPFGARAVRLYLREVRDLQSKARGISYEKKKRKRPPPQQQQQVPPPAQGASATH

SEQ ID NO: 5 - DNA, 1218 bp, Medicago truncatula
  source 1..1218: mol_type = genomic DNA
tatcagagaacaacatctactgtttcccaaattattagtacatcaaactttcatcttctcagttataaatcataatctccatcctatattctcactccctcactcactctggtcactgtctttaggaaccctagaacaattttttttctcttttcaaaacaacactggatctgaaaattaagctaaaaaaggatttaatctatggattcaattcaagattttatggactcatgtaactctgacaacagttgcagcctcaccaacagcaccataactaccaacagcagtaacaacaacatcagcaacgccatagttggtagctcttcaccttctggctctaccaccacaagcagccgctatgaaaaccagaaacgccgtgactggaacacttttggccagtacctcaagaatcaccgtcctcctctctccctctccagatgtagcggtgctcatgtgcttgaatttctccggtacttggaccaatttggcaagacaaaagtgcacacaccaatttgtccattctatggacatccaaaccctccagcaccatgtccatgtccattaagacaagcttggggtagtcttgatgcactgataggtcgtttaagggcagcttttgaggaaaatggagggaagccagaaacaaatccatttggtgctagagctgttagactttaccttcgtgaggttcgtgatcttcaatccaaagcaagaggtattagttatgagaagaagaaaaggaaacgtccaccacaacaacaaccacaacagcttcagcttcagcagcaacaaccaatgcaattgcaattgcctcttcatcttcatcatcaccatcaacatcagcttccacctccaggtgcaactcaataaaatggctattctcatattccatggaaaaaactatctttatttatctcaagtcatgaatattattaattgtctaattaattttaagtactagtagtagctagtaggtttaggttttatttatttgtcagttaaattttgtgaaagatttctggttgtcattaatttgttgtattgtatgtatgtatgtattctgcttggtgtagcagaaattaaagataagattatgaatcatcatgttttataatgacaacagttaattaatggtacttatagtaatctgttagttgaagtagcagaaaaagtggtggtggtggtatgagacttaactaattgatgtacatgatatcc

SEQ ID NO: 6 - DNA, 1647 bp, Medicago truncatula
  source 1..1647: mol_type = genomic DNA
ctttttcctgtctccatccaaacctactgtccaacttttctttcctatttcctccaaagcttaacacttctctctctctctctctctctctctctctctcaacaccatgaatgaagaatattaatatagccattagcagtaacaagtgatgatgatgatgaaacaagtttgagtgaatttgcttcacccttttaaggaaataaaaactacctaaaaaacccacagctatagttttgggaagctttctaattatatagtgttgaatgatcccatcaatctcatcacattctctacatcaaaaaaattctcctttctgcttcatcattttcaggaaccctagaagaacaagatatcaattcttggttttatcaaagaaattaacaaagtcacaaagttcaaaaacaaccttggatctgcaaagcacaaaggttgttgtattgttgaagtaaagttaagctatattttattttcatggattcaattcaagaatttatagggacatgtaacaatgaaaacttgacctgcaacttcatgaacaacaacaacaacaacactataagtacaaccacaacaagcttaacaacaacaacaacaacagcaagtggaagctcttcttcatctgcagcttcaaccatcatcaacagtcccaacagtagcagccgctatgagaaccagaaacgccgtgactggaacacctttggtcagtacctcaagaatcatcgtccacctctttccctctccaggtgcagcggtgcacatgtccttgaatttctaaggtatttggatcaatttggcaagacaaaagttcacacaccaatttgtccattttatgggcatccaaatccaccagcaccatgtccatgtcctctgaggcaagcatggggaagccttgatgcactcataggtcgtcttcgtgccgcctttgaagaaaatggaggaaaaccagaaacaaatccatttggtgctagagctgtgaggctctaccttcgcgaagttcgcgatcttcaatcgaaagcaagaggtattagttacgagaagaagaaacgaaagcgtccaccaccacaacaacaacaacaagttccaccaccagcacaaggtgcaagtgcaactcattagagatcaattctcactctcttttaataatgcacttttttttaatcatataattagggtttaataatgtgaaagctgctatgatctgaaacatatagagtgctttgtctttggatcctgtttacttgctgtgacagaagaaaaagtgttataaaatgtgagatatcacttatctgcttttaaagatatagcagacagtgctgtgtttgtactggtagtagactcatagtggcctgaaaaagttgaagcttgtgaatatacatggagtacatcatccgtgtgattctcagcaaaccactcacaacaataacagccatgacattattcatatttatatttatatttatatttatattattatatgttttgtacaacttcaatatatgttgcagcatattatgtgcctttaatcatactagattttaggacatatatataatttttctatttttgaagagttgaattttactacaattgttataagcaatttgatatatatgccacaaatggaatttaaatttgaactttgaagct

SEQ ID NO: 7 - DNA, 669 bp, Hordeum vulgare
  source 1..669: mol_type = other DNA; note = Codon optimized LSH1 protein
atggattcaatccaagatttcatggactcatgtaactcggacaacagctgcagcctcaccaactcaaccattaccaccaatagcagcaacaacaacatcagcaacgccatcgtgggttcctcgtccccgtcgggcagcaccacgacaagcagccgctatgagaaccagaagcgccgcgactggaacacgtttggccagtacctcaagaatcaccgtcctccgctctccctctccagatgcagcggtgctcatgtgctggagttcctccggtacctggaccaatttggcaagacaaaagtccacactccaatctgcccgttctacggacatcccaaccctccagccccctgtccttgccccttaagacaagcatggggcagtcttgatgccctgatagggcggttgagggcagcgttcgaggaaaatggagggaagcccgaaactaatccattcggcgctagggcggtcaggctctacctgcgggaggttcgggaccttcagtccaaggcccgaggcattagttacgagaagaagaagaggaaacgccccccgcaacagcagccgcaacagctgcagctacagcagcagcagcccatgcagctgcaattgcctctacaccttcaccaccaccaccaacatcagctcccaccgccaggggcgacgcaatga

SEQ ID NO: 8 - DNA, 645 bp, Hordeum vulgare
  source 1..645: mol_type = other DNA; note = Codon optimized LSH2 protein
atggattcaatccaggaattcatagggacatgcaacaatgagaacctgacctgcaacttcatgaacaacaacaacaacaacactatctccacgaccacaacgagcttgacgaccacaaccaccacagccagcgggagctcgtcgtcctcggcagcttcaaccatcatcaacagtcccaactcaagcagccgctacgagaaccagaagcgccgagactggaacaccttcggccagtacctcaagaatcatagaccgcctctgtccctctccaggtgcagcggtgcccacgtccttgagtttctaaggtacttggatcagtttggcaagaccaaggtccacacgccgatttgtcctttctatgggcatccaaatcccccagcgccctgcccctgtcctctgaggcaagcgtggggaagcctagacgcactcatcggtcggctgcgtgccgccttcgaagagaatggcggaaaacccgaaacgaatccgttcggcgctagagctgtgaggctctaccttcgcgaggtgcgcgacctccaatcgaaagcccggggcattagttacgagaagaagaagcggaagcggccgccaccacaacagcagcaacaggttcccccaccggcgcagggcgcctccgcgactcactga

SEQ ID NO: 9 - protein, 196 aa, Arabidopsis thaliana
MESADSGRSDPVKGDDPGPSFVSSPPATPSRYESQKRRDWNTFLQYLKNHKPPLALSRCSGAHVIEFLKYLDQFGKTKVHVAACPYFGHQQPPSPCSCPLKQAWGSLDALIGRLRAAYEENGGRPDSNPFAARAVRIYLREVRESQAKARGIPYEKKKRKRPPTVTTVRVDVASSRQSDGDPCNVGAPSVAEAVPP

SEQ ID NO: 10 - protein, 182 aa, Arabidopsis thaliana
MEGETAAKAAASSSSSPSRYESQKRRDWNTFLQYLRNHKPPLNLSRCSGAHVLEFLKYLDQFGKTKVHATACPFFGQPNPPSQCTCPLKQAWGSLDALIGRLRAAFEEIGGGLPESNPFAAKAVRIYLKEVRQTQAKARGIPYDKKKRKRPHTDTATPIAGDGDDAEGSGGAALVVTAATTV

SEQ ID NO: 11 - protein, 222 aa, Solanum lycopersicum
MDSTSRVEQPDPNIVGSSEGGTGTSSASAVTEGGQSTTVSAAPPSRYESQKRRDWNTFLQYLRNHKPPLTLARCSGAHVIEFLKYLDQFGKTKVHVTGCPYFGHPNPPAPCACPLKQAWGSLDALIGRLRAAYEENGGKPESNPFGAKAVRIYLREVRESQAKARGIPYEKKKRKRPSTSSSVATAGSAVAAEGGSSSGGGDGSGGDGVIGQQPPTDPNTTV

SEQ ID NO: 12 - protein, 207 aa, Lotus japonicus
MEATSGGASSEAAAPAVSHQAEQSSPAAVAPLSRYESQKRRDWNTFLQYLRNHKPPLTLAWCSGANVIEFMKYLDQFGKTKVHVTGCPYFGHPNPPSPCTCPLKQAWGSLDALVGRLRAAFEENGGRPESNPFGTRAVRIYLREVREGQAKARGIPYEKKKRKRSNVVTAAAVSTATVSGSGSPATAGGNDNNSNAATPSAATAATV

SEQ ID NO: 13 - protein, 223 aa, Medicago truncatula
MDPSSEGAGAPSEQLPSSTTSPPIVVQPEGSSPAPPPAPPSRYESQKRRDWNTFLQYLQNHKPPLTLVRCSGAHVIEFLKYLDQFGKTKVHVSGCPYFGHPNPPAPCACPLKQAWGSLDALIGRLRAAYEENGGRPESNPFGAKAVRIYLREVREGQAKARGIPYEKKKRKRPAVTVAAVSSSGGGNDDGGGGDGGAGDNTVGGSSGATTSTTEFSTATTTTV

SEQ ID NO: 14 - protein, 225 aa, Medicago truncatula
MKAASGGEPPPPSIPSETPPETAQPQPSSPATPTPSRYESQKRRDWNTFQQYLRNHKPPLTLALCSGAHVIEFLKYLDQFGKTKVHVIGCPYFGQPNPPSPCACPLKQAWGSLDALIGRLRAAFEENGGKPESNPFGTRAVRIYLREVKDGQAKARGVPYEKKKRKRPTVTAAAIISTAAAVNSNGPGDGDVIDGNTERIGAGVGVTTNNNTTVTPIVSVSTAIV

SEQ ID NO: 15 - protein, 209 aa, Oryza sativa
MELSPPNHESSPPTAGGGGGGGGDGAGGSSSAGASSSAGGGAATPQTPSRYEAQKRRDWNTFGQYLRNHRPPLGLAQCSGAHVLEFLRYLDQFGKTKVHTAACPFFGHPNPPAPCPCPLRQAWGSLDALVGRLRAAFEENGGRPESNPFAVRAVRLYLREVREHQARARGVSYEKKKRKKPQPADTSGGGGHPHPPPPPPPPPSAGAAC

SEQ ID NO: 16 - protein, 202 aa, Oryza sativa
MDLSPNPDSPPSGGGNGGGGGSSSSNSSPSMGAGAPQSPSRYEAQKRRDWNTFGQYLRNHRPPLSLAQCSGAHVLEFLRYLDQFGKTKVHTAACPFFGHPSPPAPCPCPLRQAWGSLDALVGRLRAAFEENGGRPESNPFAARAVRLYLREVREHQARARGVSYEKKKRKKPQQQQLQGGDSSGLHGHQHHPPPPPPAGAAC

SEQ ID NO: 17 - protein, 204 aa, Oryza sativa
MEFVAHAAAPDSPHSDSGGGGGGMATGATSASAAGASPSRYESQKRRDWNTFGQYLRNHRPPLSLARCSGAHVLEFLRYLDQFGKTKVHAPACPFFGHPAPPAPCPCPLRQAWGSLDALVGRLRAAYEENGGRPENNPFGARAVRLYLREVREHQARARGVSYEKKKRKKPPHPSSAAAAHDDAANGALHHHHHMPPPPPGAAA

SEQ ID NO: 18 - protein, 277 aa, Oryza sativa
MQGGGGGDSSGGGGGEAPRPSRYESQKRRDWHTFGQYLRNHRPPLELSRCSGAHVLEFLRYLDQFGKTKVHAAGCPFFGHPSPPAPCPCPLRQAWGSLDALVGRLRAAFEEHGGRPEANPFGARAVRLYLREVRDSQAKARGIAYEKKRRKRPPTSSSSSQAAAAAAAATSPASPAASPTPPPPPPTERSADVRPMPPEGHFFIPHPHFMHGHFLVPGGDADHHHQVSNAGNGGNTNTNTNTNTGGGGGNGDEMAVAMAAVAEAHAAGCMLPLSVFN

SEQ ID NO: 19 - protein, 219 aa, Arabidopsis thaliana
MDMIPQLMEGSSAYGGVTNLNIISNNSSSVTGATGGEATQPLSSSSSPSANSSRYENQKRRDWNTFGQYLRNHRPPLSLSRCSGAHVLEFLRYLDQFGKTKVHTNICHFYGHPNPPAPCPCPLRQAWGSLDALIGRLRAAFEENGGKPETNPFGARAVRLYLREVRDMQSKARGVSYEKKKRKRPLPSSSTSSSSAVASHQQFQMLPGTSSTTQLKFEK

SEQ ID NO: 20 - protein, 195 aa, Arabidopsis thaliana
MDHIIGFMGTTNMSHNTNLMIAAAATTTTTSSSSSSSSGGSGTNQLSRYENQKRRDWNTFGQYLRNHRPPLSLSRCSGAHVLEFLRYLDQFGKTKVHTHLCPFFGHPNPPAPCACPLRQAWGSLDALIGRLRAAFEENGGSPETNPFGARAVRLYLREVRDSQAKARGISYEKKKRKRPPPPLPPAQPAISSSPN

SEQ ID NO: 21 - protein, 214 aa, Medicago truncatula; identical to SEQ ID NO: 4

SEQ ID NO: 22 - protein, 199 aa, Lotus japonicus
MDSIQEFIGTCNSDSTCSLTNNTSMTTLTSLASGSGASSSPSASNSTAATSSRYENQKRRDWNTFGQYLKNHRPPLSLSRCSGAHVLEFLRYLDQFGKTKVHSAMCPFYGHPNPPAPCPCPLRQAWGSLDALIGRLRAAFEENGGKPEANPFGARAVRLYLREVRDVQSKARGISYEKKKRKRPPPQQSMMPPPAGATH

SEQ ID NO: 23 - protein, 222 aa, Medicago truncatula; identical to SEQ ID NO: 2

SEQ ID NO: 24 - protein, 436 aa, Pisum sativum
MDSIQDFMDSCNSDNSCSLTNSTITTNNTNNINHNNNALICSSSPSGSTATSSRYENQKRRDWNTFGQYLKNHRPPLSLSRCSGAHVLEFLRYLDQFGKTKVHTPICPFYGHPNPPAPCPCPLRQAWGSLDALIGRLRAAFEENGGKPDTNPFGARAVRLYLREVRDLQSKARGISYEKKKRKRPPPQQQHQQQQQHQPLQQLPMQLPLHLCHHQHQLPPPVVLSLHLKGNDQIKDKLMADNLQPHQQPSPPTTPQHPPQQQRLNLQLFSSVLRHQQRYENRNAVTGTSLDSTSRITALLSPSQMQRCTCTWINLANKSAHTICPFMAPNPQPCPCPLRQAWGSLDHDRSFKSALKKMEKTRYKSIWCRAVRLYLREVRDLQPKQEVLAMEKKKKSPPPQQQHQQHTQHQRFNNYQCNCLFIFVTINISFHSGATQ

SEQ ID NO: 25 - protein, 211 aa, Lotus japonicus
MDSIQEFMESCNSHDNTNCSLSNTTSNSLIVGGSSPSASVTTTSSRYENQKRRDWNTFGQYLKNHRPPLSLSRCSGAHVLEFLRYLDQFGKTKVHTPMCPFYGHPNPPAPCPCPLRQAWGSLDALIGRLRAAFEENGGKPETNPFGARAVRLYLREVRDLQSKARGISYEKKKRKRPHPQQQQQQQQPLQVLPHHHHQLQLPPPGASIRHQ

SEQ ID NO: 26 - protein, 171 aa, Solanum lycopersicum
MDSFVEVEPSNTTTNNNNITSSSSTSSSRYENQKRRDWNTFGQYLKNHRPPLSLSRCSGAHVLEFLRYLDQFGKTKIHTLICPFYGLPNPPAPCPCPLRQAWGSLDALIGRLRAAYEENGGNPEMNPFGTRAVRLYLREVRDLQSKARGVSYEKKKRKRPSQPSPPPLQSG

SEQ ID NO: 27 - protein, 218 aa, Solanum lycopersicum
MASFTELVESSNHHHHEKINIETVNNIEIISVSASSSSSAATPAPASSSSRYENQKRRDWNTFGQYLRNHRPPLTLSRCSGAHVLEFLRYLDQFGKTKVHTPMCPFYGHPNPPAPCPCPLKQAWGSLDALVGRLRAAYEENGGKPETNPFGARAVRLYLREVRDLQSKARGVSYEKKKKRKRPTPPPPIPPPQLLSTSVQLPLSSPPPSGKGALPFEL

SEQ ID NO: 28 - protein, 214 aa, Medicago truncatula
MNSLQEFESSTNNKDMMNTNPMINITNPSSSMTMTIPSSSTTSASSSSTATTSPPSTTSTTTPSRYENQKRRDWNTFGQYLRNHRPPLSLSRCSGAHVLEFLRYLDQFGKTKVHTLICPFYGHPNPPASCPCPLRQAWGSLDALIGRLRAAFEENGGKPEANPFGARAVRLYLREVRDSQAKARGISYEKKKRKRPPQPPPPPPSNNATITDLH

SEQ ID NO: 29 - protein, 214 aa, Pisum sativum
MMNSLQEFESSSNNKDMINTNPMSIIVSSSSSSSSMMTISSTTSASSSTATTTSPSSPSTTTPSRYENQKRRDWNTFGQYLRNHRPPLSLSRCSGAHVLEFLRYLDQFGKTKVHTQICPFFGHPNPPAPCPCPLRQAWGSLDALIGRLRAAFEENGGKPEANPFGARAVRLYLREVRDSQAKARGISYEKKKRKRPPQPPPPPPPSNATGTDQS

SEQ ID NO: 30 - protein, 211 aa, Lotus japonicus
MNSLQEFESSPNQDNKGISTTITNIAGSSSNLMMTMSSSASSSSSTTAATSSPPSTTPSRYENQKRRDWNTFGQYLRNHRPPLSLSRCSGAHVLEFLRYLDQFGKTKVHTHVCPFFGHPNPPAPCPCPLRQAWGSLDALIGRLRAAFEENGGKPEANPFGARAVRLYLREVRDSQAKARGISYEKKKRKRPPHPPPPPAALPPSNGARATH

SEQ ID NO: 31 - protein, 197 aa, Medicago truncatula
MNSFEEFNSSSSTKNIINFTNTTTSEDNKNISNFTSSSSSAVPPATNTNTLSRYENQKRRDWNTFGQYLRNHRPPLSLSRCSGAHVLEFLRYLDQFGKTKVHSQICPFFGHPNPPAPCPCPLRQAWGSLDALIGRLRAAFEENGGKPEDNPFGARAVRLFLREVRDSQSKARGISYEKKKRKRPQIQPPPPPSNATN

SEQ ID NO: 32 - protein, 199 aa, Pisum sativum
MNSLEEFNSSNNTQTKSIINFTTIATTSEDKNITNFTSSSSSSSAPQPANTLSRYENQKRRDWNTFGQYLRNHRPPLSLSRCSGAHVLEFLRYLDQFGKTKVHSQICPFFGHPNPPAPCPCPLRQAWGSLDALIGRLRAAFEENGGKPEDNPFGARAVRLFLREVRDSQSKARGISYEKKKRKRPQQQQQLPPPSNNAT

SEQ ID NO: 33 - protein, 208 aa, Solanum lycopersicum
MEHNQEVDSPNSVIINHHHHHNHNLDNNSMTMLAGNNNNNNNNSYLASSSSNSPTTLSRYENQKRRDWNTFGQYLRNHRPPLSLTRCSGAHVLEFLRYLDQFGKTKVHTQLCPFFGHPNPPAPCPCPLRQAWGSLDALIGRLRAAYEENGGKPETNPFGARAVRLYLREVRDSQAKARGISYEKKKRKKPNPQHSSSSSLPPPNGNSS

SEQ ID NO: 34 - protein, 248 aa, Oryza sativa
MDRHHHHHHHHHHHMMSGGGQDPAAGDGGAGGATQDSFFLGPAAAAMFSGAGSSSSGAGTSAGGGGGGPSPSSSSPSLSRYESQKRRDWNTFGQYLRNHRPPLSLSRCSGAHVLEFLKYMDQFGKTKVHTPVCPFYGHPNPPAPCPCPLRQAWGSLDALIGRLRAAYEENGGTPEMNPFGARAVRLYLREVRETQARARGISYEKKKRKKPSSAGAGAGPSSEGSPPPPGGSASGGGDTSASPQFIIP

SEQ ID NO: 35 - protein, 170 aa, Medicago truncatula
MALVSAESTNPSSYNGSDSSGGNKINRYESQKRRDWNTFCQYLRNHRPVPLSASMCNGTHVLEFLHYLDQFGKTKVHNPTCPFFGMPNPPSPCACPLRQAWGSLDALVGRLRAAYDQEINGGSSNPFGDGAVRFYLRDVRDFQSKARGVSYHKKRKRPNRNITTISQSSS

SEQ ID NO: 36 - protein, 160 aa, Pisum sativum
MELVSESTNQKGSENGIKSNRYECQKRRDWNTFCQYLRNHRPPLSASMCNGSHVLEFLHYLDQFGKTKVHNPNCPFFGMPTPPAPCPCPLRQAWGSLDALIGRLRAAYDQAVNGGNHINPFGDGVVRFYLRDVRDFQSKARGVSYHKKRKRPNRNIVAPP

SEQ ID NO: 37 - protein, 182 aa, Medicago truncatula
MDLVSSGSTNQSVESPTSGSSMTNNSTGSGSSTSTPTSSRYENQKRRDWNTFCQYLRNHRPPLSLALCSGSHVLEFLNYLDQFGKTKVHNHPCPFFGIPNPPAPCPCPLRQAWGSLDALIGRLRAAYEENGGRPETNPFGSRSVRIYLRDVRDFQAKSRGVSYEKKRKRPKPKVSATTSSAT

SEQ ID NO: 38 - protein, 183 aa, Pisum sativum
MDLVSQSTNSSLSKIESSTSGSSITTTNNSVSSGSSTPTSSRYENQKRRDWNTFCQYLRNHRPPLSLALCSGAHVLEFLNYLDQFGKTKVHHHPCPFFGIPNPPAPCPCPLRQAWGSLDALIGRLRAAYEENGGRPETNPFGSRAVRIYLRDVRDFQAKARGVSYEKKRKRPKPKITAPTTAR

SEQ ID NO: 39 - protein, 186 aa, Lotus japonicus
MDLVSESTTSTPITPPTMSISASATGSSGTVTTPTAATTPSRYENQKRRDWNTFCQYLRNHRPPLSLSLCSGAHVLEFLHYLDQFGKTKVHNHPCPFFGLPNPPAPCPCPLRQAWGSLDALIGRLRAAYEENGGRPETNPFGARAVRIYLRDVRDFQTKARGVSYEKKRKRPKPKMITTTAATAST

SEQ ID NO: 40 - protein, 181 aa, Lotus japonicus
MDLVSESTNPTTPTTTPSPNNNGLSSSRYENQKRRDWNTFCQYLRNHRPPLSMVTCDGAHVLEFLHYLDQFGKTKVHNPTCPFFGLLNPPAPCPCPLRQAWGSLDALIGRLRAAYEENGGRPETNPFGGRAVRTYLRDVRDFQAKARGVSYDKKRKRPKPNMNTSTSSSNSTSVTSSPTTT

SEQ ID NO: 41 - protein, 201 aa, Arabidopsis thaliana
MDLISQNHNNRNPNTSLSTQTPSSFSSPPSSSRYENQKRRDWNTFCQYLRNHHPPLSLASCSGAHVLDFLRYLDQFGKTKVHHQNCAFFGLPNPPAPCPCPLRQAWGSLDALIGRLRAAYEENGGAPETSPFGSRSVRIFLREVRDFQAKSRGVSYEKKRKRVNNKQITQSQPQSQPPLPQQPQQEQGQSMMANYHHGATQ

SEQ ID NO: 42 - protein, 190 aa, Arabidopsis thaliana
MDLISHQPNKNPNSSTQLTPPSSSRYENQKRRDWNTFCQYLRNHRPPLSLPSCSGAHVLEFLRYLDQFGKTKVHHQNCAFFGLPNPPAPCPCPLRQAWGSLDALIGRLRAAYEENGGPPEANPFGSRAVRLFLREVRDFQAKARGVSYEKKRKRVNRQKPQTQPPLQLQQQQQQPQQGQSMMANYSGATV

SEQ ID NO: 43 - protein, 137 aa, Solanum lycopersicum
MDVANDNPSNDVVDILSTPRLSRYESQKRRDWNTFCQYIRNHHPLMSLLQCSSIHVLEFLRYLDQFGKTKVHNSNCPFFGMINPPAPCACPLRQAWGSLDALIGRLRAAYEEHGGNSEMNPFGARSIKLFLRDVLQV

SEQ ID NO: 44 - protein, 204 aa, Solanum lycopersicum
MDFVTAQGNNFTTSGNNMVQGTNFIANSTTMIESSVPPLSRYENQKRRDWNTFCQYVRNHQPPLSLPQCTSAHILEFLRYLDQFGKTKVHNQNCPFFGLLNPPAPCPCPLRQAWGSLDALIGRLRAAYEENGGKPEMNPFGSRNVRLFLREVRDFQSKSRGVSYEKKRKRTTSSTNNNKSKIITVIDGGGDGCGTDNHNGLIID

SEQ ID NO: 45 - protein, 212 aa, Oryza sativa
MDPSGPGPSSAAAGGAPAVAAAPQPPAQLSRYESQKRRDWNTFLQYLRNHRPPLTLARCSGAHVIEFLRYLDQFGKTKVHASGCAFYGQPSPPGPCPCPLRQAWGSLDALIGRLRAAYEESGGTPESNPFAARAVRIYLREVRDSQAKARGIPYEKKKRKRSQAAQPAGVEPSGSSSAAAAAAGGGDAGSGGGAAATTTAQPGGSGTAPSAS

SEQ ID NO: 46 - protein, 238 aa, Oryza sativa
MEGGGGGADGQAQPVAQAPPAMQPMQQLSRYESQKRRDWNTFLQYLKNHRPPLTLARCSGAHVIEFLKYLDQFGKTKVHASGCAYYGQPSPPAPCPCPLRQAWGSLDALIGRLRAAYEESGHAPESNPFAARAVRIYLREVRDAQAKARGIPYEKKKRKRTQQQQPPPPPPPPPQHQPGAAAGEASSSSSAAAAAVAAEGSGSSAAAAAATSQTGGGGGGSTTTTTASAAAPTTATRV

SEQ ID NO: 47 - protein, 284 aa, Oryza sativa
MEPSPDAPRAGAAEEQPGPSSSASAPAPAASSNEEEGRHQSQAQQQVQEAQPQPLAQQAPAAAGLSRYESQKRRDWNTFLQYLRNHKPPLTLPRCSGAHVIEFLKYLDQFGKTKVHADGCAYFGEPNPPAPCACPLRQAWGSLDALIGRLRAAYEESGGRPESNPFAARAVRIYLREVREAQAKARGIPYEKKRKRGAAAAAAAPPVVVAPPPVVTAPDDATGTSGGAGEDDDDDEATHSGEQQDTTPAASPTTPPATSVGTTTAAATAAAAKGSAAKGSATSS

SEQ ID NO: 48 - protein, 181 aa, Solanum lycopersicum
MLDVYSTINSVSQNFSLSSAPAPTLPLPPPSSPPTVSRYELQKRRDWNTFGQYLRNHKPPLILARCSGANILEFLKYLDQFGKTKVHSCNCPFFGDPHPPAPCNCPLKQAWGSLDALIGRLRAAFEENGGRTETNPFGARAVRLYLKEVRDTQAKARGIAYEKKKRRNIKQRISSTINNCD

SEQ ID NO: 49 - protein, 234 aa, Medicago truncatula
MSAAVAAAASAAISSYNNSSSSSSNHSDELLTSQTMQVSVAPPLSRYESQKRRDWNTFGQYLKNHRPPLTLSRCSGAHVLEFLRYLDQFGKTKVHSENCAYFGNSHPPGPCPCPLKQAWGSLDALIGRLRAAFEENGGSQEMNPFGARAVRLYLREVRDAQAKARGIGYEKKKRKKLNQNQQNGSMTMVQNDHVHHHDDVVNSGYGHGGGFVDQYSNLNGTTSRNDAAVSYFSL

SEQ ID NO: 50 - protein, 229 aa, Pisum sativum
MSAAVAAAASAAISGCQNSSSSSSNHSEELITSQRMRVSVAPPLSRYESQKRRDWNTFGQYLKNHRPPLTLSRCSGAHVLEFLRYLDQFGKTKVHSENCAYFGNSHPPGPCPCPLKQAWGSLDALIGRLRAAFEENGGSSEMNPFGARAVRLYLREVRDAQAKARGIAYEKKKRKKVNVQNQPNGSMMVDQDHGIVHSGYGYGGGYVDQYSNFNGIASRNDAAVSYFSS

SEQ ID NO: 51 - protein, 143 aa, Lotus japonicus
MSAAVAAAAASAAISGYRNSSENQQRDVVSPYTNTPPPQMMQVSAPPPPLSRYESQKRRDWNTFGQYLKNHRPPLTLSRCSGAHVLEFLRYLDQFGKTKVHSEICPYFGNSHPPGPCPCPLKQAWGSLDALIGRLRAAFEENG

SEQ ID NO: 52 - protein, 270 aa, Oryza sativa
MDMIGMASPAESPGGGGTARPSRYESQKRRDWQTFGQYLRNHRPPLELSRCSGAHVLEFLRYLDQFGKTKVHAHGCPFFGHPSPPAPCPCPLRQAWGSLDALVGRLRAAFEEHGGRPESNPFGARAVRLYLRDIRDTQSKARGIAYEKKRRKRAAASHTKQKQQQQQLVEQAVAPPAAAAAAAALPDMETTTTTTTVPHFLFPAHFLHGHYFLAPAGEQPGGGDVAASTGGAAGAPSGGGGEDLVLAMAAAAAAAEAHAAGCMMPLSVFN

SEQ ID NO: 53 - protein, 164 aa, Arabidopsis thaliana
MTSTNTRNKGKCIVEGPPPTLSRYESQKSRDWNTFCQYLMTKMPPVHVWECESNHILDFLQSRDQFGKTKVHIQGCVFFGQKEPPGECNCPLKQAWGSLDALIGRLRAAYEENGGLTEKNPFARGGIRIFLREVRGSQAKARGVLYKKKKRLVVVGTGTSTTWT

SEQ ID NO: 54 - protein, 195 aa, Arabidopsis thaliana
MASHSNKGKGIAEGSSQPQSQPQPQPHQPQSPPNPPALSRYESQKRRDWNTFCQYLRNQQPPVHISQCGSNHILDFLQYLDQFGKTKVHIHGCVFFGQVEPAGQCNCPLKQAWGSLDALIGRLRAAFEENGGLPERNPFAGGGIRVFLREVRDSQAKARGVPYKKRKKRKKRNPMKSHDGEDGTTGTSSSSNLAS

SEQ ID NO: 55 - protein, 177 aa, Arabidopsis thaliana
MSSPRERGKSLMESSGSEPPVTPSRYESQKRRDWNTFGQYLKNQRPPVPMSHCSCNHVLDFLRYLDQFGKTKVHVPGCMFYGQPEPPAPCTCPLRQAWGSLDALIGRLRAAYEENGGPPETNPFASGAIRVYLREVRECQAKARGIPYKKKKKKKPTPEMGGGREDSSSSSSSFSFS

SEQ ID NO: 56 - protein, 167 aa, Medicago truncatula
MSSSGKDLAEGGSSTSQGITLSRYESQKRRDWNTFGQYLNNLRPPVPLSHCNSNHVLEFLRYLDQFGKTKVHLQGCMFYGQPEPPAPCTCPLRQAWGSLDALIGRLRAAYEENGGTPETNPFASGAIRVYLREVRECQAKARGIPYKKKKKSTNKGNDESSSTMHFS

SEQ ID NO: 57 - protein, 173 aa, Pisum sativum
MSSSGKDLAEGGGSSTSPQGITLSRYESQKRRDWNTFGQYLNNLRPPVPISQCNSNHVLEFLRYLDQFGKTKVHLQGCMFYGQPEPPAPCTCPLRQAWGSLDALIGRLRAAYEENGGTPETNPFASGAIRVFLREVRECQAKARGIPYKKKKKSTGNQSKGNDESSSSTMHFS

SEQ ID NO: 58 - protein, 173 aa, Lotus japonicus
MSSSGKDLAADQGSSTSPPIGATLSRYESQKRRDWNTFGQYLNNQRPPVPLSQCNFNHVMEFLRYLDQFGKTKVHLQGCVYFGMPEPPAPCTCPLRQAWGSLDALIGRLRAAYEENGGSPETNPLASGSIRVYLREVRECQAKARGIPYKKKKKSTSQSKGNDDESSSSMHFS

SEQ ID NO: 59 - protein, 176 aa, Medicago truncatula
MSTSGSNFHGKNLEQETSSTTISVATLSRYESQKRRDWNTFGQYLNNMRPPVPLSQCNSNHVLDFLRYLDQFGKTKVHVQGCMFYGQPEPPAPCTCPLRQAWGSLDALIGRLRAAYEENGGSPDTNPFASGSIRVYLREIRESQAKARGIPYKKKKKSTSQSKGSEESSSSNIQFS

SEQ ID NO: 60 - protein, 175 aa, Pisum sativum
MSSSGSNIHGKNIMEQESPTIPTTVTPSRYESQKRRDWNTFGQYLNNMRPPVQLSQCNCNHVLDFLRYLDQFGKTKVHLQGCMFYGQPEPPAPCTCPLRQAWGSLDALIGRLRAAYEENGGSPDTNPFASASIRVYLREIRECQAKARGIPYKKKKKGSQSKGNDESSSTSMNFS

SEQ ID NO: 61 - protein, 187 aa, Lotus japonicus
MSTSSGNHGKDLEQGSSSLDHYHHQHQQQQQSEPIPLSRYESQKRRDWNTFGQYLKNQRPPVPLSECNCNHVLDFLRYLDQFGKTKVHLQGCMFYGQPEPPARCPCPLRQAWGSLDALIGRLRAAYEENGGSQDANPFASGSIRVYLREVRESQAKARGIPYRKKKKNMDQSNKGIGEESSSNMHFS

SEQ ID NO: 62 - protein, 178 aa, Medicago truncatula
MSNKGKDIVEGSSRSSIGDDQNQQQQQQIPLSRYESQKRRDWNTFGQYLRNQRPPVALSNCNSNHVLEFLRYLDQFGKTKVHLQGCLFFGQTEPPGPCTCPLKQAWGSLDALIGRLRAAYEENGGLPETNPFASGSIRIYLREVRDSQAKARGIPYKKKKKKRVAIKHNGDTSNLPMQ

SEQ ID NO: 63 - protein, 185 aa, Pisum sativum
MSNKGKDIVDGSSSRSSTSTIGDGNGGGDHHQQQQIPLSRYESQKRRDWNTFGQYLRNQRPSVALSQCNSNHVLEFLRYLDQFGKTKVHLQGCLFFGQTEPPGPCTCPLKQAWGSLDALIGRLRAAYEENGGLPETNPFASGSIRIYLREVRDSQAKARGIPYKKKKKKRIPIKHNGDTSNLPMQ

SEQ ID NO: 64 - protein, 192 aa, Lotus japonicus
MSTKGKDMAEGSSRSSASGAGADADARVRAVDDDQQQQKQLPLSRYESQKKRDWNTFGQYLRNQRPPVALSQCNSNHVLEFLRYLDQFGKTKVHSQGCLFFGQTEPPGPCTCPLKQAWGSLDALIGRLRAAYEENGGLPETNPFASGSIRVHLHEVRDFQAKARGIPYKKKKKKRNVINHNGDSSSNLPMPP

SEQ ID NO: 65 - protein, 179 aa, Lotus japonicus
MSNKGKDIAEGSSPASDHHHHQQQQQQPLSRYESQKRRDWNTFGQYLRNQRPPVALSQCNSNQVLEFLRYLDQFGKTKVHVQGCLFFGQAEPPGPCTCPLRQAWGSLDALIGRLRAAYEENGGLPEANPFASGTIRLYLREVRDSQAKARGIPYKKKKKKRNVMKAMTEGVSSSSLPMQ

SEQ ID NO: 66 - protein, 178 aa, Solanum lycopersicum
MSSSDIRGKDLAEGSSRSPGRDQPPSRYESQKRRDWNTFNHYLKNQRPPILLPHCHSNHVLEFLRYLDQFGKTKVHLLGCMFYGQPDPPAPCTCPLRQAWGSLDALIGRLRAAYEENGGSSETNPFASVGIRVYLREVKECQAKARGIAYKKKQKKLANSPSKGDHDDASCPGFLTFS

SEQ ID NO: 67 - protein, 192 aa, Solanum lycopersicum
MNPSTIVMTKELSAGSSRSGGEQLQNNNPAPLSRYESQKRRDWNTFGQYLKNQRPPVPLSQCNCNHVLEFLRYLDQFGKTKVHLHGCVFFGQPDPPAPCTCPLRQAWGSLDALIGRLRAAYEENGGSPENNPFGNGAIRLYLREVKECQAKARGIPYKKKKKRKLNNNSIKPIGAGVGASADQHKNLMQANI

SEQ ID NO: 68 - protein, 185 aa, Medicago truncatula
MSSSSSQIENSSLHPSSSKSNHHQQQQPLSRYESQKRRDWNTFNQYLKSQNPLVPLSHCNFNHVLDFLRYLDQFGKTKVHFQGCIFFGQPTPPAPCACPLKQAWGSLDALIGRLRAAYEENGGSSETNPFAGGAIRVYLREVKDSQQKARGIPYKKKKKKKKNYQIKGTTQADQGACVKQQDSYN

SEQ ID NO: 69 - protein, 164 aa, Pisum sativum
MKKLQRVRYESQKRRDWNTFGQYLKSQNPPVPLSNCSFNHVLDFLRYLDQFGKTKVHLNGCIFFGQPTPPAPCACPLKQAWGSLDALIGRLRAAYEENGGSPQTNPFAGGAVRVFLREVKDSQQKARGIPYKKKKKKKKSYQVKGSTSSTAQTQQSAYVNQQDS

SEQ ID NO: 70 - protein, 184 aa, Medicago truncatula
MSSSMQNDTVQASSSSSRPGGSDQQPTAAVAPLSRYESQKKRDWNTFGQYLRNQSPPVSLSQCNFNHVLEFLRYLDQFGKTKVHLHGCIFFGQPDPPAPCTCPLRQAWGSLDALIGRLRAAYEEHGGSPENNPFGTGAIRVYLREVKECQSKARGIPYTKKKKKRSQLKGTHNTTSKSFNQLAS

SEQ ID NO: 71 - protein, 183 aa, Pisum sativum
MSISMQNDTVQASSSSSRPGATADHPSVVAPLSRYESQKKRDWNTFGQYLRNQTPPVSLSQCNFNHVLEFLRYLDQFGKTKVHLHGCIFFGQPDPPAPCTCPLRQAWGSLDALIGRLRAAYEEHGGSPENNPFGTGAIRVYLREVKECQSKARGIPYTKKKKKRSQIKGTHNNSSKSFKQLAS

SEQ ID NO: 72 - protein, 178 aa, Lotus japonicus
MSAEMQNDSTQASSSSSRPSAAPLSRYESQKRRDWNTFGQYLKNLTPPISVSQCNFNHVLDFLRYLDQFGKTKVHLHGCIFFGQPDPPAPCTCPLRQAWGSLDALIGRLRAAYEEHGGSPETNPFGSGAIRVYLREVKEGQAKARGIPYTKKKKKKRSLITGSHDQVQPSKSFKQLAS

SEQ ID NO: 73 - protein, 175 aa, Lotus japonicus
MSSQLQNGSPHGSSSSSQPPSNQQQHHHHRQVQSQPQQQPLSRYESQKRRDWNTFGQYLKNRTPQVPLSQCNFNHVLDFLRYLDQFGKTKVHSHGCIFFGQPSPPAPCACPLRQAWGSLDALIGRLRAAYEEHGGSPDTNPFGAGAIRLYLREVKECQAKARGIPYKKKKRKKNN

SEQ ID NO: 74 - protein, 185 aa, Solanum lycopersicum
MSSSEQKREVGGREGEGSSSMTLTQSDHHHQLSPPPQLSRYESQKRRDWNTFGQYLKNHKPPVPLPQCNYNHVLDFLRYLDQFGKTKVHLNGCVFFGQVEQVGPCTCPLRQAWGSLDALIGRLRAAYEENGGLQETNPFANSAIRIYLREVRDSQAKARGIPYKKKKKKRKIQINSSNNNEVNST

SEQ ID NO: 75 - protein, 205 aa, Solanum lycopersicum
MSNDQIIIEGEGGGGGGEGSSSRSKTTILIAPSDDHHHHHQLPPVPPQLSRYESQKRRDWNTFGQYLKNQRPPVPLSQCNYNHVLEFLRYLDQFGKTKVHLHGCPFFGQPEPPGPCTCPLRQAWGSLDALIGRLRAAYEENGGLPENNPFASGAIRVYLREVRDFQAKARGICYKKKKKKRKMQNKPTSSNAHEPTTTTFQFQSS

SEQ ID NO: 76 - protein, 213 aa, Solanum lycopersicum
MMSSEKIREVGEGSSSSGGAISIIATPLNNHHRQSSSSSLSTLAPTPASSSAPQLSRYESQKRRDWNTFGQYLKNQRPPISLPQCNYNHVLDFLRYLDQFGKTKVHLHGCLFFGQPEPPGPCTCPLRQAWGSLDALIGRLRAAYEENGGLQETNPFASGAIRVYLREVRDSQAKARGIPYKKKKKKKRPNLQIKASNNNDGATSANFQLQSTT

SEQ ID NO: 77 - protein, 191 aa, Arabidopsis thaliana
MSSDRHTPTKDPPDHPSSSSNHHKQPLPPQPQQPLSRYESQKRRDWNTFVQYLKSQNPPLMMSQFDYTHVLSFLRYLDQFGKTKVHHQACVFFGQPDPPGPCTCPLKQAWGSLDALIGRLRAAYEEHGGGSPDTNPFANGSIRVHLREVRESQAKARGIPYRKKKRRKTKNEVVVVKKDVANSSTPNQSFT

SEQ ID NO: 78 - protein, 154 aa, Lotus japonicus
MSAAAAAAAAAGAAVSYRSNSPENYNQTEEFSPYTPPQTTQFYPVARPRFSRYESQKRRDWNTFCVYVKNHHSPSCHCDGGEIVLGFLRYMDQFGETQIHSENCEYYGDSYPPGPCPCPPKQSWDALDGIVGRLLVAFEENGGSPEMNPFGKEW

SEQ ID NO: 79 - protein, 155 aa, Lotus japonicus
MSAAAAAAAAAGAAVSYRSNSPENYNQTEEFSPYTPPQTTQFYPVARPRFSRYESQKRRDWNTFCVYVKNHHSPSCHCDGGEIVLGFLRYMDQFGETQIHSENCEYYGDSYPPGPCPCPPKQSWDALDGIVGRLLVAFEENGGEGVSQGSERCSS

SEQ ID NO: 80 - protein, 184 aa, Lotus japonicus
MSAAAAAAATGAILGYRNSSRSKQREEFLPFTPPKMMHAVSPVALPRISRYESRKRRDWNTFCVYLRNQYHPSLTPYSCSDEHVLEFFRYMDQFGETKVHFETCAYFGDSYDPPGQCHHCPLKQAWDSLEGIVGRLHAAFEESGGSPEMNPFGTRMVRVHLMEVRVQDRSKGIGYEKKKRSMSL

SEQ ID NO: 81 - protein, 174 aa, Lotus japonicus
MSAATAAAAAVAAAATSQTTIIPSSSSARTSAVAPPRMLQVSPVALPRFSRYDSLKRHDWTTFRQYLTNHHPSLTLSRCGGEIALDFLRYLDRFGETKIHTENCEYFGDCYPPGPCPCPLKQSWDGLDGIVGRLRVAFEENGGLPEMNPFRGNAVRVYLREVRDAQARARGRGQ

SEQ ID NO: 82 - protein, 276 aa, Oryza sativa
MSSSSAAALGSDDGCSPAELRPSRYESQKRRDWQTFTQYLAAHRPPLELRRCSGAHVLEFLRYLDRFGKTRVHEPPCPSYGGRSPSAAGPVAAAAAACQCPLRQAWGSLDALVGRLRAAYDERHGRAGEPDAVAGAGAVATDSTSSSSAAAANPFAARAVRLYLRDVRDAQAMARGISYHKKKKRRGGNMNGARGGGGGGARAGVNDGDATAPPVAVTPGLPLPPLPPCLNGVPFEYCDFGSVLGGAHGAHGGHGGGGGGFYGAGVYLPFLYNTFS

SEQ ID NO: 83 - protein, 234 aa, Pisum sativum
MLLHFSGIGAMPWIIMSEIKCNVDDSAVGVPINAACLGIFRDHNSNHLGSFSLNIGQGNALFVEITGAIMAIEITKDNHWTHLCGAHVIEFLKYLDQFGKTKVHVSGCPYFGHPNPPAPCACPLKQAWGSLDALIGRLRAAYEENGGRPESNPFGAKAVRIYLREVREGQAKARGIPYEKKKRKRSAVTVSAVSSSGGGANDDSGGGNGGAGDNTVVGGGTTATTTTAANASTS

SEQ ID NO: 84 - protein, 185 aa, Pisum sativum
MRILQKVHRPYMKLASGGRTTTCQPSSTPQPELHQRRPTPKADTSHKSDEELEPRSNTKVPRRLGCPYFGQPNPPSPCACPLKQAWGSLDALIGRLRAAFEENGGKQESNPFGTKAVRIYLREVREGQAKARGIPYEKKKRKGSTVTTAAIVSTAAAVVTGDGNTNRIELGVTPYNSVSLSAEIV

SEQ ID NO: 85 - DNA, 5500 bp, Synthetic construct
  source 1..5500: mol_type = other DNA; note = LSH1 promoter region including a putative NIN binding site at nt 5,260
ccaagcacgagtcttgggacaaggcgaagtggttaatcgtaaagttgttttagacattgaagggcgggcggcatctaccattttatgaatcttatggcgagttcacttgcatatggtacgactgaagtctgcatttttgttggatttagatctaactgttgagctttggcgacttttaattttttgctgacttgttcgaatcgtggcgtcattttaatgtaattttcaataggcgaaccttgaacggcgccttataactttatgatgtaattttatgaagtaatctatgattggcaggccttgaatggcgccctaatattttatttgtaaccttatcaattttaccttagtatcttgatgcgaatgttgctttttgaattcttctaattttggtattttcgttttattcagtatggtcctaaaagtttatttgtttgtatgagccatctcccacttagggaggtttgggcgaagatgtttcccataacttctctatgagaaggattcatatacttcccacttagggaagacttctctatgagaaggctttcatatgctctccatttaatgagggcttctctatgagaaacctttcgtatgattcccacattaaggaagacttctctgtgagaaggctttcgtatgcttcccattttaaggaaggctattatgtgagaaggctttcgtatgctttccacttaaggaaggcttctctgtgagaaggctttcgtatggttcccactttaaggaaggcttctctgtgagaaggcccattttaaggaaggcttctttgtgagaaggcttttgtatgcttcccacataaggaaggtttctctctgagaaggctttcgtatgcttttcttgtttgatttttgaagatttatttgatttaaactgtagttggaactgtttttagaaagtaggatgcctcattaaaaacctacaaaacaaatctcaaaagtcaaataagggttaagataactcagcgtttagtctttgtgttcttagtggcaagattgttgcttccatatgcacttcctttttcacatattaattatggtgtgttgttttggcgagttcattttcaacttcgcccttggtgagcctgtctgcatcatattcatgtctatgattcatcataataatccttccacacttccgaagtaaataaataaaggagtcttaatcacccaatcgtatttaatgagaactaattactcgttgtcttctcttattaactcatccatattttctactctccccttcctattcttttcattctcttactagatttgaaataaccttcttatttgattaacttatgcttttaatccttttctttcttctattagattggaactaagttttcctactacttctcttaaatcgaaattcgctacttccttattagatttaaactaacttgcccatttcgttcttttattttatactcctcctttttctcctactaacttgaattttaatttatttgatactttatttacttcctcctatgcatttggaactaagtgttctgctcaattttattcttctacatgggttggcgctattaatccattatttatgattttaccaaacagttagttcagttcgcaaagtaaatggtacatgaaaggggtttccaggaaaaagaggtgggtcacttggacctcttcaatactaagactttcctcgccagaatccctcatatgtgaccttttaacaatactacactttacttctgccacggggtacctagagttgtctttcttttattaacttggtcctacctcttgtgttttttactcatcttgctttatttattaaattgtactaaatcacacagcgagaattaatagcaagtgaaacaaatgaagttaaatctaattgtccataagactgattatctacaaattatgtatatgcaccaaaaaacaattgcaaaataaacaaaattgataagagccttcgaaaaagcatgaagacttaagttggtaagtaatgttttacatatcaactctacgcgtaaaaatacagtgaagatccatttttatacttacactatgaacctggcccaaaggcgaatcttagtattttagtaaattttaataaaatattatatatgttctccaatttgaattattataatatttattttataataaaaatagtaaaggtttgccctttgacaagcttcatattagacatacgcatatatatatatatatatatatatatatatatatatatatatatatatatatatatatataattatttaattttttaggtcaatctaatatggacatttacaaaattttctaaatatgaacctaaatattttttgcaaaaacataatttagaaataataaaagggaaataaacacaatattttactaacatattttgaaaaaaccagtttataaaagaaaatctaatagtaagtccacctttgaaccgtattcatattcgacacttcatatcttttttaccaaattataatattttttatatatataattgataaatttctttaaaaaataaattgattcatttcattgacaaaagaaaatagcctctcaacttttttttataaatttttttttggatgaattactaaccattctaattgttaaaaggatggattctttcaaaaaaattgttaaaaggatggatttgattaattcattaacaatttattatatgataggattagccgaatggtagataatttgatgcaattgtcatttttcttttaaggatgcaattgtcatatctaatatgtagaagaaatgcatatgaatttaattttgacaaaaaaatacacatgaatttgaatgctttcctatctaagattatattgaaattatattatttgtatgtcgtaagcacaagagtagttgaagaaattttattattccctaaaatatgattattattcatcgatacatttataaagaatgtcccatggtcaccctttttttttaaggacccttttattattatatttcaccttttacaaaatgtagtcaaaattgtgaacataattaagtgcaacccgaccatctttcaaggaagaattttgtatgtctaatctttaaaccaaaatgtctgtcatattatggtaatttcatttccatgtatatatatatatatatatatatatatatatatatatatatattggctgtgagaaacaaagatagggacaaaacagcataagaaattaaagttttatttctctctctagggaagggaaataaaatggtagtaaaaagaaagggggaaattaatagaggcataccagtcaatgagaaaggaactgtcattcaaagtttcaacctatagtgtgaagaaaacagaatcatgttctttaccccaatttttacctttcatactttttcctgtcgccatctcaaactactgtacatcccttgtttttctattttcataaaaaagccctagcaagtagcaacagcatccatttctctgtctattcaccaacataataaaggaatattaatattgccattaatataagtgatgaaagtctgagtttgcaccacccttttaaggcaaaaaataaataaaagttaaatcctaaaaaaaccacataattcaaaagaaaattgactgtactttgaacaattacatctcttatacttatattttgtttctactctttttttttttttttttttctctattgtttttaacctataaaaaaaaaatatatattatcttaaaagttgttacaaaatggttttacagctatcattattcaaagtcctttttcttcttccgtattacttattttgacaacttttcagttgtccaacactcaatatttattcgacacaagtactatttaaacaaaccatatcatttttttcctctaatattgatcctagttgtttagtgagagaaactatattttctctttaagttctatttgtcctttgttcattgaaaatatgtggtttaggtcattttttgactgtaatcactaagtaagccgcatttcttttttctctttctttcaattctaaataggtgattttcacatatatcaagttttttcctatgttttttctcgtaatttcatcgtctgagtgtgactttgtgttgttagtcgtcttgtgtaacattgattgattttgcgtccgttgcggtgttcgtttgcttcctattgaaagatcggcttgaatcaattgcagattcaatcaatgatgtagacgccaatgttgcagatttgaaagtatatatattctagtgactgagattttatcatattatttctagctattttgttgttgtatagctattttatcataaccatcatttatatttttaacgatgttgaacatgttattgtatcggatgtgctttatcagattgaataaaaggatatcgttttctttttgtcaaaaaattgtaccatttagtttttgaattatttcttctctcatcaaattacatcaataattataaatgatatattttaatatgatgttgaaagtaattgtcaatttttaagttgttcaaataacaccactcttcttctcgtattgctcaacaaatacgactcacttgtagatggaatttctaacactcacacattcaagaaatcaatgatcatttaatcaaaatcatacactcatgctccaaaactcaatattttaatttataaaattaaaatatgtattttcagatcaattgatattgattggatgatcatcaatgtctcgaatgcaaaattctccatcatgttaaaaggattaatccaatttcagtgctcaacaaaaaatacaagagtaataccctttaaatacaaatacaacttggtaaaattctctttcgacacctcttatcttcacaaaatacaataatatgagtaataatatttacacaaccaattttaaaatgtttttaataaaatacaacgtaagtacgagagacacgtcataaaatagatgcataattattatttgagaaatgatatttgtacaaccaatttctaacaattttttgacaaatttctctctcatattcacgatatattcttattttctctttacctttttctctctattgattttgaccaatcaaaataaagaaaaacaaagttgtcataaaaattatttagaaaaagatggacaaatatcatgaaaattatttaaaaagagatgtacaactatcgctgctcttattatttgtccaacaacaaatagtaaagaggaaaagtggaaaagttcagtcacccctcccatcaatctcatcacatatcctcaattaacataaaaaatcttcttgcttcaccacttgaagaggtccataccatataccatctatctcttcaatttctctctctacacaaaaaaacaaactagtctgtaatatcaatcaaccaaatcagagtttgtttccttttcattttctttcaaggtagtttttttttctaaaaatgaaaagacattaaaaaatattattattattttgt

SEQ ID NO: 86 - DNA, 201 bp, Synthetic construct
  source 1..201: mol_type = other DNA; note = LSH1 5 UTR
tatcagagaacaacatctactgtttcccaaattattagtacatcaaactttcatcttctcagttataaatcataatctccatcctatattctcactccctcactcactctggtcactgtctttaggaaccctagaacaattttttttctcttttcaaaacaacactggatctgaaaattaagctaaaaaaggatttaatct

SEQ ID NO: 87 - DNA, 323 bp, Synthetic construct
  source 1..323: mol_type = other DNA; note = LSH1 intron
gtacataccaacaaaaaaaatatgaaatgagtctctaattcaatctttattttttattaacaagttttggtggaagagaaatatttagaagtagagattgaatcctgaaacctggaacatctcttgttctttgataaggaaataagtgtagaatttcctctctttttttttctaactaattattagggtatgtctatacgaaagatcgatctagggttcaatttttgctagtaaaatttctccatattcaagagagaaaaaatgatttatgaaacctaatcttgtcacatttttttggttgaattgttaatattgtgatgtag

SEQ ID NO: 88 - DNA, 348 bp, Synthetic construct
  source 1..348: mol_type = other DNA; note = LSH1 3 UTR
aatggctattctcatattccatggaaaaaactatctttatttatctcaagtcatgaatattattaattgtctaattaattttaagtactagtagtagctagtaggtttaggttttatttatttgtcagttaaattttgtgaaagatttctggttgtcattaatttgttgtattgtatgtatgtatgtattctgcttggtgtagcagaaattaaagataagattatgaatcatcatgttttataatgacaacagttaattaatggtacttatagtaatctgttagttgaagtagcagaaaaagtggtggtggtggtatgagacttaactaattgatgtacatgatatcc

SEQ ID NO: 89 - DNA, 3000 bp, Synthetic construct
  source 1..3000: mol_type = other DNA; note = LSH1 downstream terminator region
cactgatgaaacaaaagctactcatcatagttatatattcatgattctataatttttttatttttttatttttgtgatttattcataattacatatttacatgtatgtatgctcttcatcttctttgatttttttttttttatggttaaaatttctcagagaataaataggatgtttgagattcgtattctaacgaactaagctcactaaaactatgttcttcatcttctattatgttttatttggtacaatatgattttttatttatctatgggtaaatatttatgggaagtctgaccaacaacttagaacttcttcaatatctcttgttagctaaccaactaaattatctataatatgcagattcgtatattatatgactctaaattgattaattattgtcgggtgcatctaatatggatttttaaaaatactttaagatgtactcttataatttttaataaattttaaacaaatatagagttatctaatgtgaactaggtcaaacgatgaatctttttattttttaataagttttaataaaaaaaaaaaaagaattcttcattttctagtattataaaattcatttttttactaaaaagagtataggtccgctatttaattattttttagagattcatatcagatatagccataaaatattaatcttttcacttttttttttactattataaattttattattttactaaaaatgctaatggttcatctttaaatcttttgaatgaggtccagattagagaaatatccatttttatcatcattttgggttccatcattatttctttcacttgaatccctcaattttgctagcaaattcattgacctctcttgagtaattgtttgaacttaatttgaagctatagctaggtagccaattccgtacaacttcatgtcaatcatattattggttgcatcttttatatactatgctattttctctcttcattattttccttgtaatttactaattttatctagtacttcactcttgttaaaataaaataaaaaataaaaaaaatctatagtactttacatattgtctccttgaaatgtgataggggtccttttattaaagaatgatagggccctttaagcttaatcagaagacatgagacgagtttcttttggtttctttctcccatcaattgaagagttactgctgaccgtttacctttggtcaatttgattgatgatatgatactatatactgcttcatatgttttgcagtagtgctgtcacttcacttgaagagaaatgacgaaataaaggaaaaactaatggcagacaatgtaattaataattcctgtcactaaagtttatttttatttacttacacatttatattagctatatacatatatttacttgtcaatattatacatatacaccgatcataggtaattcatctccttggtttaccaagactttcaagtaatagttataacattaattatcgttttcaattcagttaggttatgccatccatattagagacatttcaaatctcactctacctactagtgaatgtccattgaatgaatggtatactaactccgtctctacatatacataattcatttgactacattagttgaataaagaaagttgatttgtcctctcaaaaatgaagttgatttgtagtattatatgttttgttaataattgatatttatttaaatttaggtttattataattttgaaccccttaattatacctttagatcattttttagaggtcaatattggacccaccttttttttaagaagatatattggacacacctataaataataggtaaaacaattaattatttttttaagaatcaatgatagttcagtgaagaaatgctaaacgggcacatcttaagttaactcgtaaatcaatttttaacctaattttaacttgtattttaagatgatttttacgttattaccccgtgttcaattaaacctgtttaattaagagataaattttaaaaatcgttcctcctattcctataacaattttctcttattttatttccaacacaacaataaatagataaacataaaaaaaaaaatacaattttctcatattttgtttccaacacaacaataaacagataaacataaaaaaaaataaaaaatcactcttattaacaaattcttcctattatttccatttaatcaaaatgttatctttatatataaagttaacccgtaaatcaatttttaacctaattttaacctagattttatgatgccttttacgttattaccccgtgttcaattaaacttgtttaattaagagataaactttaaaaatcgttcctcctattcctataacaattttctcatattttatttccaacacaacaataaacagataaacataaaaaaaaaatacaatatcctcctattttgtttccaacacaacaataaacagataaacataaaaaaaaaaaatcactcttattaacaaattcttcctcttatttccatttaaacgatttcatgtcatctcctactttattttttgaacttgcgtaagtaaaatgtataatactcttcatcttttatttattttaggattttttttacaattacttattcttatatattagtcgaaatgacccgtgcgacgcacgggtgtaaagtctagtaaaaactaaacagaaatgctaaaaggacacggaaaattgatgaaaatttgttgttctcttgttttttgttgtgtattttagtaatccgtaaaattgataaaaagttgagaaaataaagatagagttatagggtgccatttaattaattttattgtcttcatttaatgtccaataaaaattgtatacttaaaattatctcaagagttgttcacctttttttttaaacaatgatttttttttttcacttaaaattttaaaacccaatacttacctttatgcctcgtattttcttgggtaatcctacctgttttaacatagaatacagagagatcat

SEQ ID NO: 90 - DNA, 5000 bp, Synthetic construct
  source 1..5000: mol_type = other DNA; note = LSH2 promoter region including putative NIN binding site at 5000 bp
gattgtaaatgaactataaaagcatccataacggtagttttttaatggtttaattgcatttttggccccctatcttttcaaaagttgtgattttggccctctaattaattaaaatataaaacaacatcttatgtattggatatttggcagttttggcccccaagcccacttttgacttagtcttcgctgacgtggcacccacaatggtcgacacgtggcacctcacttaagggatgtgggtgccacgtcagcgaagactaagtcaaaagtgacattgggggccaaaattgtccaatatctaatacatagggggctgttttgtatttttaattagttagggggccaaaagtgcaattaagccttccttaatagggactccttaaatggaccccacgttacacatcacttatttataatattttaataaaagtatctattaaggattcaatgactccaatgagagtacactaataaggactctaaagaaaagtacttaaataggccccactaacttacatcattaaacaaaaataatttttttattaaattataaattacctcataatatttaattataatcctttaaaaaaattaaattataaattttttatttattcgtatgaacttaaatttctgccatatttgctaataatttgaaaatggaaaagttaacattacaaattttatattccttcgttaaataataataataataatttgcataaattatattttattttaaattatgataaatagataaaataatattgaaaacaactattaattttggtgactccttaaaatattactcctttctacaagtgggatcaaatagaacatattttattacttttttaaacgtggatccgttagttattcaggagagagagggtccttatatagttacatgtttcctaacgggaactcattttttcttcctccaatggagggtccttaactgggagtggttcttgtttagtaaagacccccattgaagatgcaatttgctgtaattgtataaatgtaaaacaatttataggtattatagggacatatagactataggtacaaggtattatttttttggtaagaattactaggtattataggaatcatgtcatctttgaacattgaaacataaataatagattggttcctctaaaccgagtaatatctactatgattaacgaatcaaaagcaaatattataagtgtacaataaaaaaattataagtgttttcgtaagtttaatttagttggtgggatcaatacataatatatgtaggggtccgtatttaaatggaggtaattcacttcaaacaaaaggaacaaagaaacgcctacaaaaataggctaaacaacaagaacagaagaaaccaaggaaatcaaagaaacccaaacaaacaaaagaaaacaagagatataaatatacggaaaatacttgggtgacatagagttgggtgacattggtaacattggtcaatcacaatgccacaatgcttgtgtttctctctctttcacaagcattgtggcattataattgatcaatgtcaccaatgtcacctaactctatgtcacctaagtgttacccataaatatacctcaaaggagagagaaacatgtacagggatagtacacacaaagctaaccaaaattagcaaaacactcttaagctagctacacaagaaacatgaaaatacgaaaaggagccaagtaacaaagcgccaccaagcctctattggagcaagtaaggggagagttcctccacaacccactcatactaagaacaagagaagccattatacctacccaaaaatcactttcaagaatagaattgaatatgatccacccattgctcaacacttataacttaattagagaagataatatcattcaaagtcttcgaatttcgaaataccctacactataacatactagacaagggaaaacccttttctattacgaagattaattttaaaaaaggtataaaaattgtgaacaaattgaacagattcgaaggcatggcggtcatccactgaaattattttaaaatattgtatgacgtgcggagggaaaaattgtacctaccaaaaaggtgaacaattgactcctcaaagaatcaccatgaaatacatattgcatatgaggtatcatgaataaccttgcgattgagaagattgacacgggtgggcatcctatccaacgataactaattagacttatttatgtttgattaatttgtagatcctgaacaatttgataatttttaaacatatcactaaactaaaaattaggagtaatcaagtttttttaacttttttaattaagtcatttcgttaaattaccattagactaccgttactcaacacctacatgacatattacattatgaaaaatgttgtgtggtgcgaatcatctatcgtgcaaaacccacgaattatcttttttaacttttacaattcaacttctatatatttcaaaaaaaaacaattcaacttctatagaaaatgaagtgcggcagccatgtgctctcatactaaatgccatggtgcatcgatataggctttttatacattttttttttgtgtgaaatattgaggcattttatacatatttagtttactgttcattttgattaaaataataataataataaaattcttatatatattttttaaaatataggtttaattgtacttttgatcttttttgttgttgttaaaattgtaattttagtcctttatttttagtgctatattaacaaccctaaagacatttgttaaggaaattaatgtcttacaatattaaattcatcgcttttctccatttttcaatgtaatatttctatttttgttctcttaacaaatgtcttcaggtcactctttagtatttatcttatttttaattgtgcaattatggtcttccatttttttttattaatatatgattttgaccatctaatttgttgatgtggtagtaaactatgtaatctatgatttttaccattttcatcaactctcttgtagagttctctctagaatctctttgtaacctcattgcaatttcactggagaacaacctcaaccattaattagaatagccctgccgtattagaaaactttttatacacattacgttgattgaatatacattgattcaatatacactagattacttattaattgaactatattcacaatcttgaaaataatgatcatgtatacacccagcaacaatgtaaacatttttaaaacttgaaatgaaattaaagaagataatagtttgccttttcaaaaaacaaagtattttaccaaaagaataaaaaagaacaaacaaaaaagactatatagcttagtatattgggcatttaataaccccttcgtgcaagtcacgcgaatattcattcgcaccatttagcaacagtgcaacactgttttgtcaagaaaaaaacatcattatcattaaatattatttactaaaatttactaaaacacaatattgaaatgcccaaatctttaatttctcgttgtggatagaaaggtaaacaaacaatgtggggcatggtgcttcagattgaaaattgaaatgactatgtgaggtaagaatagtttaatcaataattacgtctaaaaattaaattcaagttttcttaaacgatttattctttaataaaatttattaatcatttgagtccaatcatttaacttaattataagttggagatttactctcaacaaataaaaaaacttcatataaaatgaagcaaaataaaaaaggagataaattatttggcttcagatgctcaaaatctcagcttataaattgacatggttgctccaggtggaaaattaaactttgattgtaaaatttaataaaattgacatgtcaagaaattagacatttgacatgaactgcatttccctcaaaaaaaaaaaaaaaaaattgcatgtttggttttaagtcgttttaaaagttttagttggtcttttatgttttaaaatggtacattatgttttaaaaattttaaattggtttaaaacattactcacaataaaataagttagtctcttccatccaataatattgagtttaataaagcaaaaaaaaaattaaaaattgattttgtaaagagaactgatattagtacaaccattctatgataacttttgtacaactttctctttcatattcacatgatggacttatcctctctcttcctttttctctctccattgtttttgacaaataagaagagagaaaaacaaggttgtcactaaagttgtcatgaaaggatgtacaaatatcattgctcttttgtaaataagttaaattttaaaactcgtattctcaacctagatatctaaccgaccaagtgacaaggggactatcttattccaacataagtaatgttttgtactaacataaaacttttaaaacataaataaccaacttaaaacatttaaaacatgaaatctaaaatatccataagagatcaaacatgcttaaccttaaaaaacaattaatagtataattttttatgggattattagcacaaacttgttttattctattctctcctaataaattttaccttattaaaactttatatcgataaattttcaatatcaaacattaaatagaataaaaatgttataaataatcattttttgaaagctgttataaataatcatgaaaatatattgtctctattatatttaaagaacattaattgatagcatttttcatatcgaggaattcattaaaaaattcctttaatttttaaaattattattatttgaatattaataaggtgatgacatgagaagtggacaaaggaggtgtcagaaattaaagtttttatcttttgaaagaggaacaggaaaggttaaaaaggtaaactgaggggtattatagtcaattaaaatgaactgtcattccaaactacagagtcaagaaaagtaagcagaatcatgtttattccctcatttttaccccttcaaa

SEQ ID NO: 91 - DNA, 558 bp, Synthetic construct
  source 1..558: mol_type = other DNA; note = LSH2 5 UTR
ctttttcctgtctccatccaaacctactgtccaacttttctttcctatttcctccaaagcttaacacttctctctctctctctctctctctctctctctcaacaccatgaatgaagaatattaatatagccattagcagtaacaagtgatgatgatgatgaaacaagtttgagtgaatttgcttcacccttttaaggaaataaaaactacctaaaaaacccacagctatagttttgggaagctttctaattatatagtgttgaatgatcccatcaatctcatcacattctctacatcaaaaaaattctcctttctgcttcatcattttcaggtacatatttccctatcctattcctaaaatatacataaaaatagtttgtaacataccaattaattaattctttcacttttgtccaaggaaccctagaagaacaagatatcaattcttggttttatcaaagaaattaacaaagtcacaaagttcaaaaacaaccttggatctgcaaagcacaaaggttgttgtattgttgaagtaaagttaagctatattttattttc

SEQ ID NO: 92 - DNA, 333 bp, Synthetic construct
  source 1..333: mol_type = other DNA; note = LSH2 intron
gtatatgtagtacaaatttaattataaattatgaaattaatcaccatgcaaatgcaacctcgctttcttttcaactgcatctctcactcttcccaagttttgaatagtagttgcaattacatcacaattttttatattagagaaaatcatagtctaatatcgtcgatgcttgcttgaatttgactttttatccgacatggtttaaatctcgtttaaacagttgtaaaataatcattagtgtcatacatagctataaattgcggtagctatgattcgaattttagctgtatataatttttaatactattttggttaatttatcatggtgtttag

SEQ ID NO: 93 - DNA, 531 bp, Synthetic construct
  source 1..531: mol_type = other DNA; note = LSH2 3 UTR
agatcaattctcactctcttttaataatgcacttttttttaatcatataattagggtttaataatgtgaaagctgctatgatctgaaacatatagagtgctttgtctttggatcctgtttacttgctgtgacagaagaaaaagtgttataaaatgtgagatatcacttatctgcttttaaagatatagcagacagtgctgtgtttgtactggtagtagactcatagtggcctgaaaaagttgaagcttgtgaatatacatggagtacatcatccgtgtgattctcagcaaaccactcacaacaataacagccatgacattattcatatttatatttatatttatatttatattattatatgttttgtacaacttcaatatatgttgcagcatattatgtgcctttaatcatactagattttaggacatatatataatttttctatttttgaagagttgaattttactacaattgttataagcaatttgatatatatgccacaaatggaatttaaatttgaactttgaagct

SEQ ID NO: 94 - DNA, 3000 bp, Synthetic construct
  source 1..3000: mol_type = other DNA; note = LSH2 downstream terminator region
acatttgtggcaagtagtcatttccatacatagtgatcgatgtggataacattatttggaatgattcccatatatgtatatattgatatgatatatcgatgtgattatttgttcatatctacaaaagaggagagtatattgtgctcttttcttatcactttctctctttttaatggccacttagagtgttgtcctctccaaatgtattttgttaccaaaaaaaaagcataatatatgatgatcatactaataagttctgtttcaaactgacaagctttattcatttctctccccattgagactaacagttgaccgttaaatgcataactttgcattgatgatcatactgattcatgtgtgcagcagtagtgctgtcatttcaaattatcaaaaggaaattgccaaaaaaatattattaatggcagacaatgtaattcattcctaaatgactaagccttcaattattagtttttattggtaacttcaattaattctacttttttaaccatccattgctaaggaatggagttttatagttgatagctcgattggttgctaatagtttctaaggaagggagttttatggttgatattttgattgggagttaatagtaaaacggtagtgctatatctcgcaagaaattttagtacgatgggactacgaaatacctcttagccacttaaattctctcttggccggaaaaaacaggattgttgttcctgtattttttttgtatgctgcggtttggtcaaagaattatgtaaaattaaatggacaaaatcttgccaacacattcatttaatttcaagaatcattgtttcacactacctctagcattatccatagtaaaatcttggtccaagaaaaggggaggaggtgattacgagttaaagtcatggtatgtacatgtgatatagagcatctttattacaaattataaaacaaatgattttgacattaaatagagataaattctaagacatcgatgcagagaaactcgggagggtatggttctctctcctctccctaaccgtttttctccggtgaatttatctaccttcggtgcagctccggtggccgttttttcaccgttttattcgattatcttcgtcgtttcacatgcgtttcttgcctccatcatctgcatcgcttctctccgcgggctttttgcctcttagtgtttggcggtggtggttttagctctctggtgtggtggcgcttggttctcgaagtggttacggttgtctctttgcttctaattgtgattttcgttctctatgtcttcaatctgtccttggccatgcttagcttctatggagacagtggtacctacggcggcaaaatcgtcggattctggtaaatccttcgctcaagctctatcttgttccggtgaccttcaattgactaaattgccacccaaaattgtgatgggaacatcggtgcgtgtcaaaatttcgcaagctgagtatgaatttggtgttgctgcgtgtaagtgtaacctccatggcagagtcgctctgcacaagggggatcctccattgacaacactttcatagaaacagaagctttccggattgtggcctgatatgcacaattggaatcttacacctcttggtaagggttttttcgactttaacttcagttcaattgaagacatgcacagagtttgggcattgagggtggtgaatatgaaacctgggttccttcgtttctactgctggacaaaagatttcaagcctcaagctcaaatgcaaactcatgctcaaatttgggtcaggttgatgcacttaccccaagaatattagaggaagacaaccctgtttgagattgcttcaggcttaggtacccctcttgcaattgatgaggtaactcatagcagacgttttgggattttttccagagttcttgtggatgtggacctatctgagcagctctttgaaactgttgttgtggaaagggaagatcatgctctgtccattgatgtcttttatgagaagcatccttccttctgtgctaattgcaaaaccttaggacatagcttaatcacacaaacaatactgaagtgcctgctagaatgaaaactaggactgatttgaatggaaagaaacatgcttctaacacatccaatgctttcaaaatcgtatttttcaagaaacaaggtggacttgatgtgcatagcaagaaaaatcctgcaagtgggattctcaatgttgaagaaattgctactgattatgttcatcatgagattcctcagtctgagatcgttcctgccgactctatcaatatcagtaaatccactgactctcggatggaacttgaaaaccggcctttctctactcaaaagaatctagagaatgaaggagctaaagataatgagaatgttacagtaatcatgaaggatatgagttcaggtcctgctattacattgcaagatgcgaattcagatcctgctttggctcttcacaactcatttgacattctacatgaggatagtgaactttcttttagggagacttctctagtggatcagggactaagtaacagctcctatgatatgcagcatgctatcttggagcctgctgatgtagctaaaagtgtgagtgcaactgttactaactttttggatactagcttgatcatagctccaaacgtggccttgaatactgtgttttcagacaagaataacatacttgtgcagcaccccataccataacctatcactacttattacacttctttaacaagtgacaagtttcctgttagcacagttattcggatgcagaccactaacatacactctgaagctagcctcaaaagtgtccaaattctgaagaaattttggagggatattgatgatgatgaagaagattcagcctctaatgatagcctccaggaagcggaatctgatgtctccaaatacttggctcataatgtagagcttgctactaacaggaaagctcagaggcagaaaaaggggaagtccagtgctggtaaaatct